(19) United States
(12) Patent Application Publication
(10) Pub. No.: US 2021/0323985 A1
(43) Pub. Date: Oct. 21, 2021

(54) SHIELDING AGENTS AND THEIR USE

(71) Applicant: ENDOCYTE, INC., West Lafayette, IN (US)

(72) Inventors: Christopher Paul LEAMON, West Lafayette, IN (US); Iontcho Radoslavov VLAHOV, West Lafayette, IN (US)

(21) Appl. No.: 17/277,806

(22) PCT Filed: Sep. 19, 2019
(86) PCT No.: PCT/US2019/051903

§ 371 Date: Mar. 19, 2021

Related U.S. Application Data
(60) Provisional application No. 62/734,690, filed on Sep. 21, 2018.

(51) Int. Cl. C07F 9/38; C07C 275/16; A61K 51/04; A61K 31/662; A61K 31/17

(52) U.S. Cl. CPC C07F 9/3808; C07C 275/16; A61K 31/17; A61K 31/662; A61K 51/0497

(57) ABSTRACT

The present disclosure relates to compounds useful as shielding agents for PSMA therapies. The present disclosure relates to methods of treating PSMA expressing cancers with one or more radiotherapeutics agents in combination with one or more shielding agents. The present disclosure relates to methods of imaging using one or more imaging agents containing a radionuclide in combination with one or more shielding agents. The present disclosure also relates to methods of making shielding agents.

### CROSS-REFERENCE TO RELATED APPLICATIONS

This application claims priority under 35 U.S.C. § 119(e) to U.S. Provisional Application Ser. No. 62/734,690 filed on Sep. 21, 2018, the entire disclosure of which is incorporated herein by reference.

### TECHNICAL FIELD

The present disclosure relates to compounds useful as shielding agents for PSMA therapies. The present disclosure relates to methods of treating PSMA expressing cancers with one or more radiotherapeutics agents in combination with one or more shielding agents. The present disclosure relates to methods of imaging using one or more imaging agents containing a radionuclide in combination with one or more shielding agents. The present disclosure also relates to methods of making shielding agents.

### BACKGROUND

Prostate specific membrane antigen (PSMA) is a type II cell surface membrane-bound glycoprotein with ˜110 kD molecular weight, including an intracellular segment (amino acids 1-18), a transmembrane domain (amino acids 19-43), and an extensive extracellular domain (amino acids 44-750). While the functions of the intracellular segment and the transmembrane domains are currently believed to be insignificant, the extracellular domain is involved in several distinct activities. PSMA plays a role in the central nervous system, where it metabolizes N-acetyl-aspartyl glutamate (NAAG) into glutamic and N-acetyl aspartic acid. Accordingly, it is also sometimes referred to as an N-acetyl alpha linked acidic dipeptidase (NAALADase). PSMA is also sometimes referred to as a folate hydrolase I (FOLH I) or glutamate carboxypeptidase (GCP II) due to its role in the proximal small intestine where it removes γ-linked glutamate from poly-γ-glutamated folate and α-linked glutamate from peptides and small molecules.

PSMA is named largely due to its higher level of expression on prostate cancer cells; however, its particular function on prostate cancer cells remains unresolved. PSMA expression is highly restricted in man, present in only salivary gland tissue, renal tissue small numbers of cells in the small and large intestine. PSMA is over-expressed in the malignant prostate tissues when compared to other organs in the human body such as kidney, proximal small intestine, and salivary glands. Higher PSMA expression is associated with high grade, metastatic and castration resistance disease. Tumor expression in prostate cancer is typically 100 to 1,000-fold higher. Unlike many other membrane-bound proteins, PSMA undergoes rapid internalization into the cell in a similar fashion to cell surface bound receptors like vitamin receptors. PSMA is internalized through clathrin-coated pits and subsequently can either recycle to the cell surface or go to lysosomes. It has been suggested that the dimer and monomer form of PSMA are inter-convertible, though direct evidence of the interconversion is being debated. Even so, only the dimer of PSMA possesses enzymatic activity, and the monomer does not.

PSMA is also expressed on the neovasculature of other tumors, such as thyroid cancer, renal clear cell carcinoma, transitional cell carcinoma of bladder, colonic adenocarcinoma, neuroendocrine carcinoma, glioblastoma multiforme, malignant melanoma, pancreatic duct carcinoma, non-small cell lung carcinoma, and soft tissue sarcoma, breast carcinoma. These cancers represent a large range of different tumors with different histological subtypes, growth rates and cell cycle times. In some cases, the cancers are imbedded within normal tissues having variable radiation tolerances. In addition, hypoxic areas of larger deposits may also lead to radio resistance. These and other factors are known to result in different intrinsic response to traditional external beam radiation therapy.

Though the activity of the PSMA on the cell surface of the prostate cells remains under investigation, it has been recognized by the inventors herein that PSMA represents a viable target for the selective and/or specific delivery of biologically active agents or combinations of biologically active agents, including drug compounds to such prostate cells. One such drug compound is the compound of Formula I

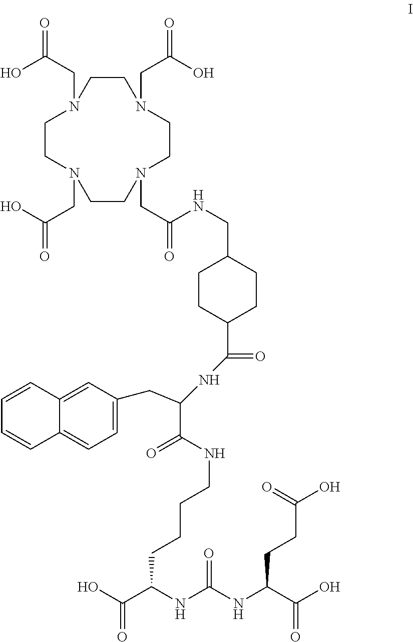 [SMILES: O=C(O)CC[C@H](NC(=O)N[C@@H](CCCCNC(=O)C(Cc1ccc2ccccc2c1)NC(=O)C1CCC(CNC(=O)CN2CCN(CC(=O)O)CCN(CC(=O)O)CCN(CC(=O)O)CC2)CC1)C(=O)O)C(=O)O]

wherein ¹⁷⁷Lu is complexed to the compound to provide I-Lu, or ²²⁵Ac is complexed to compound Ito provide I-Ac, useful for the treatment of cancer as described in WO2015/055318. Compounds I-Lu and I-Ac can be prepared according to the methods described in WO2015/055318, incorporated by reference for the preparation of Compounds I-Lu and I-Ac, as described in Example 3 and Example 5.

Another such drug compound is Compound Ia

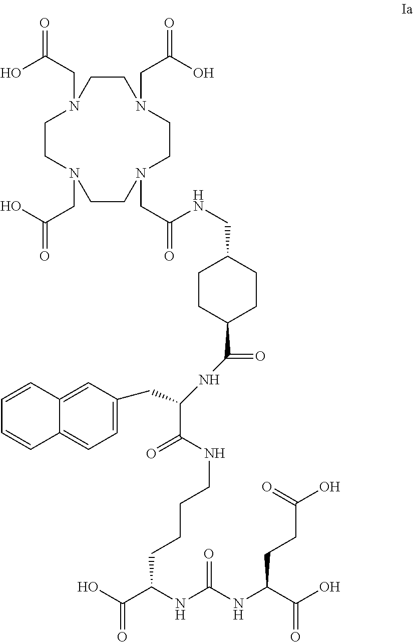 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O]

(a.k.a. (3S,10S,14S)-3-[(naphthalen-2-yl)methyl]-1,4,12-trioxo-1-[(1R,4S)-4-[[2-[4,7,10-tris(carboxymethyl)-1,4,7,10-tetraazacyclododecan-1-yl]acetamido]methyl]cyclohexyl]-2,5,11,13-tetraazahexadecane-10,14,16-tricarboxylic acid) wherein ¹⁷⁷Lu is complexed to compound Ia to provide Ia-Lu, or ²²⁵Ac is complexed to the compound to provide Ia-Ac, useful for the treatment of cancer as described in WO2015/055318. Compounds Ia-Lu and Ia-Ac can be prepared according to the methods described in WO2015/055318, incorporated by reference for the preparation of Compounds Ia-Lu and Ia-Ac, as described in Example 3 and Example 5.

Compound I or Ia can be described as a small molecule that specifically binds to PSMA (prostatic specific membrane antigen) which is expressed on the surface of prostate cancer cells. Compound I or Ia can be characterized as composed of a pharmacophore ligand, glutamate-urea-lysine; a chelator, DOTA (able to complex ¹⁷⁷Lu and ²²⁵Ac); and a linker connecting the ligand and the chelator. Without being bound by theory, it is believed that the urea-based pharmacophore ligand allows the agent to bind to, and be internalized by PSMA at the site of disease. It is further believed that the binding of I-Lu, I-Ac, Ia-Lu, or Ia-Ac can lead to internalization through endocytosis which can provide a sustained retention of the ligand and its bound radioactive cargo within the cancer cell.

Previous radioligand therapy (RLT) used in the clinic includes ¹³¹I in thyroid cancer, and elements emitting alpha radiation, such as ²²³Radium or ⁸⁹Strontium, for the treatment of bone metastases.

¹⁷⁷Lu has a half-life or 6.7 days. It emits a combination of 0.5 MeV energy consisting of negatively charged Beta particles (electrons) that travel chaotically through tissues for approximately 20-80 cells or 0.5-2mm and cause predominantly base damage and single strand breaks. At high dose these lesions can interact to convert sublethal damage (SLD) or potentially lethal damage (PLD) to irreparable, lethal damage. ¹⁷⁷Lu also emits 113 Kv and 208 kV radiation which can be used for imaging.

²²⁵Ac has a half-life of 9.9 days, and in contrast emits 8.38 MV energy alpha particles. Only 0.5% of energy is emitted as 142Kv photon emissions. The majority of radiation particles are therefore positively charged, and about 8,000 times larger than β particles. Furthermore, the energy from these particles is deposited over relatively short distances (2-3 cells). As a result, there is dense and severe tissue damage in the form of double strand breaks with multiply damaged sites that represent irreparable lethal damage. This is called High Linear Energy Transfer (LET) or densely ionizing ionization and it delivers 3-7×more absorbed dose than β.

The type of cellular damage inflicted by either isotope (¹⁷⁷Lu or ²²⁵Ac) is expected to be different due to the difference of the characteristics of each warhead. ¹⁷⁷Lu is believed to provide a longer path length of radiation and therefore can be effective in delivering radiation to adjacent cells. The preponderance of single strand breaks, especially in the presence of oxygen, provides the opportunity to repair sub lethal damage (SLD) and or potentially lethal damage (PLD) providing the optimal conditions for normal tissue repair. On the contrary, ²²⁵AC delivers extremely powerful, high LET radiation, and the potential for repair of normal tissue is much more limited. The radiological biological effectiveness of alpha radiation is at least 5 times that of beta irradiation and administered doses the relative biological effectiveness (RBE) has to be taken into account. With ²²⁵Ac therapy, the type of DNA damage inflicted does not require the presence of oxygen so it will also be more effective in hypoxic tumor regions. A possible disadvantage of ²²⁵AC therapy is that the short path length can lead to large amounts of damaging radiation deposited only within a short distance of 2-4 cells.

Another such compound is the PSMA-imaging conjugate 2a

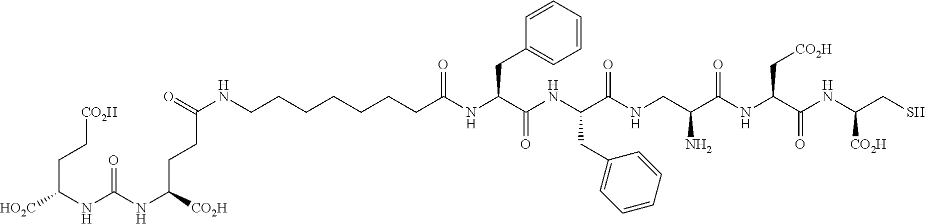 [SMILES: N[C@@H](CNC(=O)[C@H](Cc1ccccc1)NC(=O)[C@H](Cc1ccccc1)NC(=O)CCCCCCCNC(=O)CC[C@H](NC(=O)N[C@@H](CCC(=O)O)C(=O)O)C(=O)O)C(=O)N[C@@H](CC(=O)O)C(=O)N[C@@H](CS)C(=O)O]

(a.k.a. (2R,5S,8S,12S,15S,29S,33S)-8-amino-12,15-dibenzyl-5-(carboxymethyl)-1-mercapto-4,7,11,14,17,26,31-heptaoxo-3,6,10,13,16,25,30,32-octaazapentatriacontane-2,29,33,35-tetracarboxylic acid). ^{99m}Tc (or similar radioactive metal isotope) can be complexed to the conjugate 2a, and is useful for the imaging of a patient as described in WO2009/026177. PSMA imaging conjugate 2a can be prepared according to the methods described in WO2009/026177, incorporated by reference for the preparation of PSMA imaging conjugate 2a, as described in the examples.

Another such compound is the PSMA-imaging conjugate 4

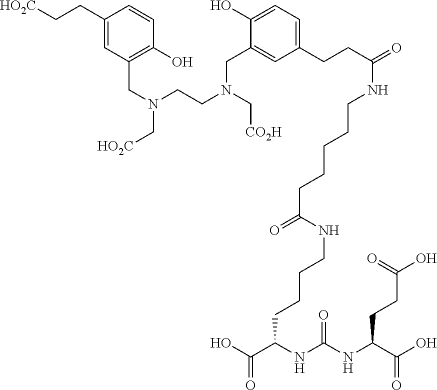 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

(a.k.a. 4,6,12,19-Tetraazadocosane-1,3,7-tricarboxylic acid, 22-[3-[[[2-[[[5-(2-carboxyethyl)-2-hydroxyphenyl]methyl](carboxymethyl)amino]ethyl](carboxymethyl)amino]methyl]-4-hydroxy-phenyl]-5,13,20-trioxo-,(3S,7S)) wherein ⁶⁷Ga or ⁶⁸Ga (or similar radioactive metal isotope) is complexed to the conjugate, useful for the imaging of cancer as described in Eder M, Schafer M, Bauder-Wust U, Hull WE, Wangler C, Mier W, et al. ⁶⁸Ga-complex lipophilicity and the targeting property of a urea-based PSMA inhibitor for PET imaging. Bioconjug Chem. 2012; 23: 688-97. PSMA imaging conjugate 4 can be prepared according to the methods described in (Eder, 2012), and (Eder, 2012) is incorporated by reference for the preparation of PSMA imaging conjugate 4, as described in the examples.

The use of PSMA conjugates containing radionuclides, such as ¹⁷⁷Lu and ²²⁵Ac for the treatment of disease or ^{99m}Tc and ⁶⁷Ga or ⁶⁸Ga can lead to of target delivery of the radionuclide. Without being bound by theory, it is believed that such off target delivery can occur in tissues, other than tissues containing PSMA expressing cancer cells, where PSMA is expresses. For example, biodistribution experiments using radiolabeled PSMA compounds and imaging conjugates, such as those described herein, can show accumulation of the radionuclide in tissues such as the kidney. It would be advantageous to develop compounds useful as shielding agents to be administered in methods of treating or imaging a patient using radiolabeled PSMA compounds and imaging conjugates.

### SUMMARY

In some embodiments, the present disclosure provides compounds useful as shielding agents of PSMA. In some embodiments, the present disclosure provides a method for treating a cancer in a patient in need of such treatment comprising, administering to the patient a therapeutically effective amount of a compound containing a radionuclide (a radiolabeled therapeutic), such as ¹⁷⁷Lu or ²²⁵Ac, in combination with one or more shielding agents of the disclosure. In some embodiments, the present disclosure provides a method for imaging in a patient comprising, administering to the patient an effective amount of a conjugate containing a radionuclide (an imaging conjugate), such as ^{99m}Tc, ⁶⁷Ga or ⁶⁸Ga, in combination with one or more shielding agents of the disclosure.

In some embodiments, the present disclosure provides a method for treating a cancer in a patient comprising administering a therapeutically effective amount of Compound Ia-Lu or Ia-Ac in combination with a an effective amount of a shielding agent, such as those shielding agents described herein. In some embodiments, the method comprises administering a combination of Ia-Lu and Ia-Ac.

In some embodiments, the present disclosure provides a method for imaging a patient comprising administering an effective amount of an imaging conjugate, such as imaging conjugate 3 or 4, labelled with a radionuclide such as ⁶⁷Ga, ⁶⁸Ga or ^{99m}Tc, in combination with a an effective amount of a shielding agent, such as those shielding agents described herein.

In some embodiments, the present disclosure provides use of Compound Ia-Lu or Ia-Ac for treating a cancer in a patient in combination with an effective amount of a shielding agent, such as those shielding agents described herein. In some aspects, the use comprises administering to the patient a therapeutically effective amount of the Compound Ia-Lu, and a therapeutically effective amount of the Compound Ia-Ac, in combination.

In some embodiments, the present disclosure provides use of an imaging conjugate, such as imaging conjugate 3 or 4, labelled with a radionuclide such as ⁶⁷Ga, ⁶⁸Ga or ^{99m}Tc, in combination with an effective amount of a shielding agent, such as those shielding agents described herein for imaging a patient.

In some embodiments, the present disclosure provides use of Compound Ia-Lu or Ia-Ac, in the preparation of a medicament useful for the treatment of a cancer in a patient, in combination with an effective amount of a shielding agent, such as those shielding agents described herein. In some aspects, the medicament comprises a therapeutically effective combination of Compounds Ia-Lu and Ia-Ac.

In some embodiments, the present disclosure provides use of an imaging conjugate, such as imaging conjugate 3 or 4, labelled with a radionuclide such as ⁶⁷Ga, ⁶⁸Ga or ^{99m}Tc, in the preparation of a medicament for use in combination with an effective amount of a shielding agent, such as those shielding agents described herein in imaging a patient.

In some aspects of these embodiments, the cancer is a PSMA expressing cancer. In some aspects of these embodiments, the compound or imaging conjugate is at least about 98 percent pure. In some embodiments, the cancer is selected from the group consisting of a glioma, a carcinoma, a sarcoma, a lymphoma, a melanoma, a mesothelioma, a nasopharyngeal carcinoma, a leukemia, an adenocarcinoma, and a myeloma.

In some aspects of these embodiments, the cancer is selected from the group consisting of lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast carcinoma, triple negative breast cancer, metastatic breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung carcinoma, cancer of the adrenal gland, soft tissue sarcoma, cancer of the urethra, cancer of the penis, prostate cancer, metastatic castration-resistant prostate cancer (mCRPC), thyroid cancer, transitional cell carcinoma of the bladder, colonic adenocarcinoma, neuroendocrine carcinoma, glioblastoma multiforme, malignant melanoma, pancreatic duct carcinoma, chronic leukemia, acute leukemia, lymphocytic lymphomas, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, glioma, brain stem glioma, pituitary adenoma, and adenocarcinoma of the gastroesophageal junction. In some aspects of these embodiments, the cancer is a primary or secondary brain cancer. In some aspects of these embodiments, the cancer is prostate cancer. In some aspects of these embodiments, the cancer is metastatic prostate cancer.

In some aspects of these embodiments, a combination of Compounds I-Lu or Ia-Lu and I-Ac or Ia-Ac is administered in a parenteral dosage form. In some aspects of these embodiments, the parenteral dosage form is selected from the group consisting of intradermal, subcutaneous, intramuscular, intraperitoneal, intravenous, and intrathecal. In some aspects of these embodiments, the therapeutically effective amount of I-Lu or Ia-Lu is from about 2 GBq to about 13 GBq. In some aspects of these embodiments, the therapeutically effective amount of I-Lu or Ia-Lu is from about 4 GBq to about 11 GBq. In some aspects of these embodiments, the therapeutically effective amount of I-Lu or Ia-Lu is from about 5 GBq to about 10 GBq. In some aspects of these embodiments, the therapeutically effective amount of I-Lu or Ia-Lu is from about 6 GBq to about 9 GBq. In some aspects of these embodiments, the therapeutically effective amount of I-Lu or Ia-Lu is from about 6.5 GBq to about 8.5 GBq. In some aspects of these embodiments, the therapeutically effective amount of I-Lu or Ia-Lu is from about 7 GBq to about 8 GBq. In some aspects of these embodiments, the therapeutically effective amount of I-Lu or Ia-Lu is about 7.4 GBq. In some aspects of these embodiments, the total dose of I-Lu or Ia-Lu ranges from about 15 GBq to about 200 GBq. In some aspects of these embodiments, the total dose of I-Lu or Ia-Lu ranges from about 25 GBq to about 185 GBq. In some aspects of these embodiments, the total dose of I-Lu or Ia-Lu ranges from about 35 GBq to about 150 GBq. In some aspects of these embodiments, the total dose of I-Lu or Ia-Lu ranges from about 40 GBq to about 100 GBq. In some aspects of these embodiments, the total dose of I-Lu, or Ia-Lu is about 44 GBq. In some aspects of these embodiments, the maximum duration of treatment of a subject is about 19 to 23 months.

In some aspects of these embodiments, the therapeutically effective amount of I-Ac or Ia-Ac is from about 1 MBq to about 20 MBq. In some aspects of these embodiments, the therapeutically effective amount of I-Ac or Ia-Ac is from about 4 MBq to about 14 MBq. In some aspects of these embodiments, the therapeutically effective amount of I-Ac or Ia-Ac is from about 5 MBq to about 10 MBq. In some aspects of these embodiments, the therapeutically effective amount of I-Ac or Ia-Ac is from about 6 MBq to about 8 MBq. In some aspects of these embodiments, the therapeutically effective amount of I-Ac or Ia-Ac is from about 1 MBq to about 4 MBq. In some aspects of these embodiments, the therapeutically effective amount of I-Ac or Ia-Ac is from about 2 MBq to about 3 MBq. In some aspects of these embodiments, the therapeutically effective amount of I-Ac or Ia-Ac is about 2.5 MBq.

In other aspects, the methods and uses described herein further comprise imaging PSMA expression by the cancer. In some aspects of these embodiments, the step of imaging occurs before the step of administering. In some aspects of these embodiments, the step of imaging occurs after the step of administering. In some aspects of these embodiments, the imaging is performed by imaging wherein the imaging is selected from the group consisting of SPECT imaging, PET imaging, IHC, and FISH. In some aspects of these embodiments, the imaging is performed by SPECT imaging.

In some aspects of these embodiments, imaging as described herein comprises administering to the patient a PSMA ligand-imaging conjugate of the formula 2

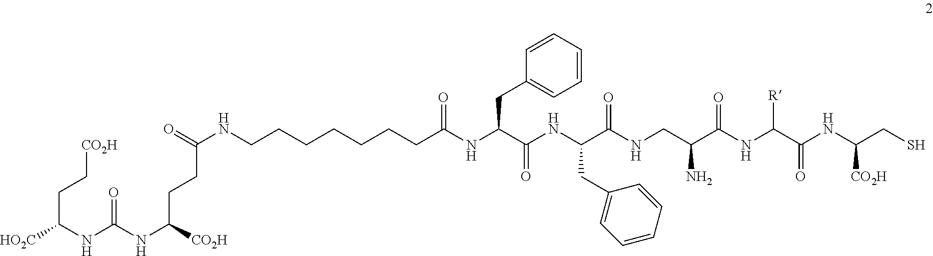

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein a radionuclide is bound to the conjugate.

In some aspects of these embodiments, imaging as described herein comprises administering a PSMA ligand-imaging conjugate of the formula 3

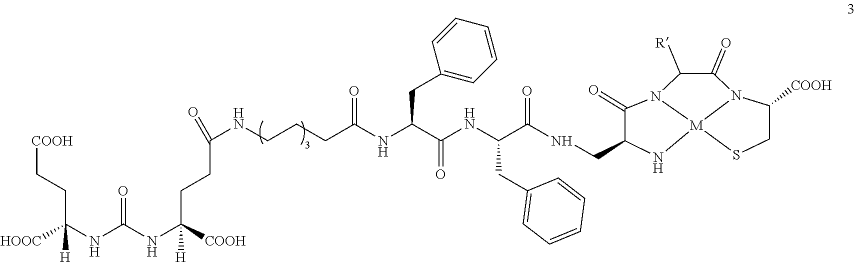

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein M is a cation of a radionuclide. In some aspects of these embodiments, M in the conjugate, or a pharmaceutically acceptable salt thereof, is selected from the group consisting of an isotope of gallium, an isotope of indium, an isotope of copper, an isotope of technetium, and an isotope of rhenium. In some aspects of these embodiments, M in the conjugate, or a pharmaceutically acceptable salt thereof, is an isotope of technetium.

In some aspects of these embodiments, the PSMA ligand-imaging conjugate is of the formula 2a

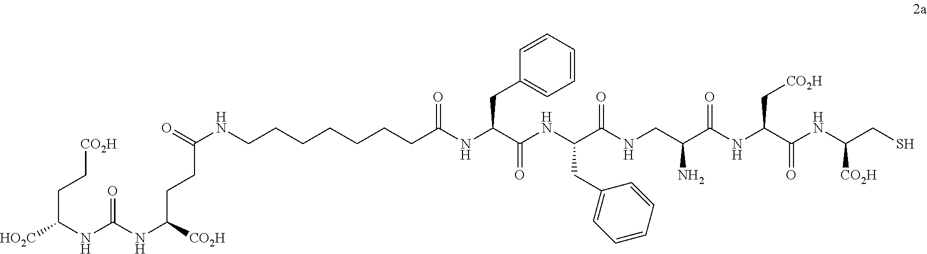 [SMILES: N[C@@H](CNC(=O)[C@H](Cc1ccccc1)NC(=O)[C@H](Cc1ccccc1)NC(=O)CCCCCCCNC(=O)CC[C@H](NC(=O)N[C@@H](CCC(=O)O)C(=O)O)C(=O)O)C(=O)N[C@@H](CC(=O)O)C(=O)N[C@@H](CS)C(=O)O]

or a pharmaceutically acceptable salt thereof, wherein a radionuclide is bound to the conjugate. In some aspects of these embodiments, the PSMA ligand-imaging conjugate is of the formula 3a

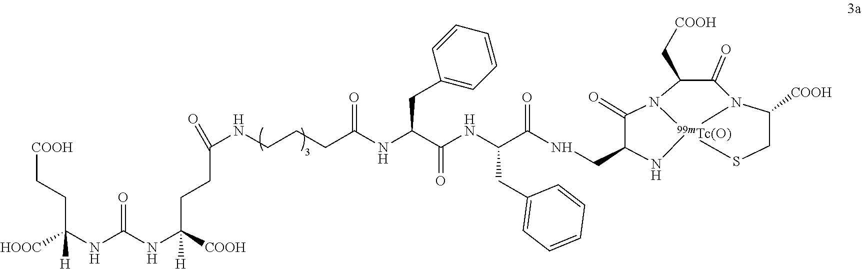

or a pharmaceutically acceptable salt thereof.

In some aspects of these embodiments, imaging as described herein comprises administering to the patient a PSMA ligand-imaging conjugate of the formula 4

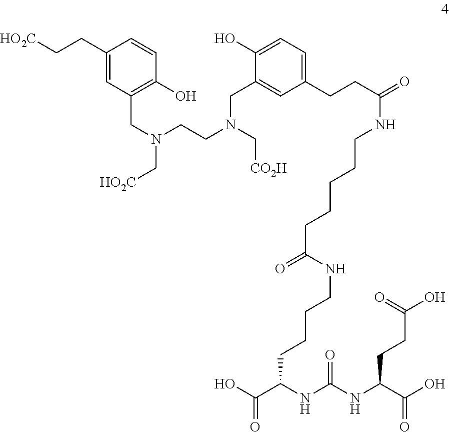 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

or a pharmaceutically acceptable salt thereof, wherein a radionuclide is bound to the conjugate. In some aspects of these embodiments, the radionuclide is ⁶⁷Ga or ⁶⁸Ga.

In some aspects of these embodiments, imaging as described herein comprises detecting the compound of the formula I-Lu or Ia-Lu administered for the purpose of treating.

In other aspects, the methods and uses described herein further comprise determining the PSMA status of the patient by imaging In some aspects of these embodiments, the step of determining occurs before the step of administering. In some aspects of these embodiments, the step of determining occurs after the step of administering. In some aspects of these embodiments, the imaging is SPECT imaging. In some aspects of these embodiments, the PSMA status of the patient correlates with a clinical benefit to the patient. In some aspects of these embodiments, the clinical benefit is selected from the group consisting of inhibition of tumor growth, stable disease, a partial response, and a complete response. In some aspects of these embodiments, the clinical benefit is stable disease. In some aspects of these embodiments, the PSMA positive lesions indicate functionally active PSMA.

In some aspects of these embodiments, determining as described herein comprises administering to the patient a PSMA ligand-imaging conjugate of the formula 2

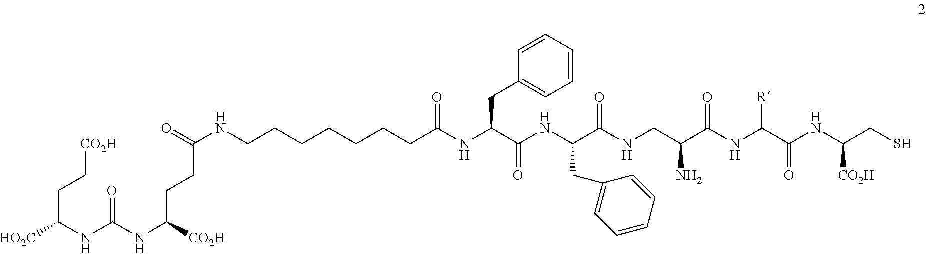

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein the conjugate is bound to a radionuclide.

In some aspects of these embodiments, determining as described herein comprises administering a PSMA ligand-imaging conjugate of the formula 3

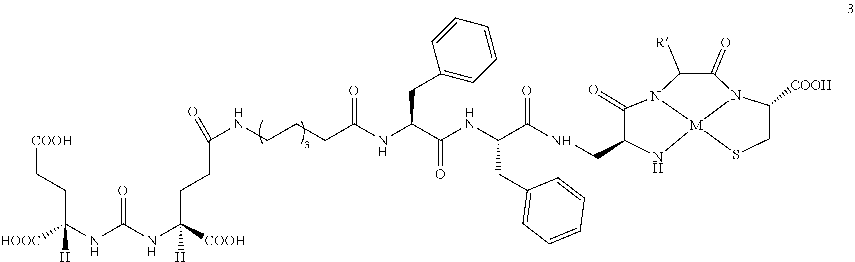

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein M is a cation of a radionuclide.

In some aspects of these embodiments, M in the conjugate, or a pharmaceutically acceptable salt thereof, is selected from the group consisting of an isotope of gallium, an isotope of indium, an isotope of copper, an isotope of technetium, and an isotope of rhenium. In some aspects of these embodiments, M in the imaging conjugate, or a pharmaceutically acceptable salt thereof, is an isotope of technetium. In some aspects of these embodiments, the PSMA ligand-imaging conjugate is of the formula 2a

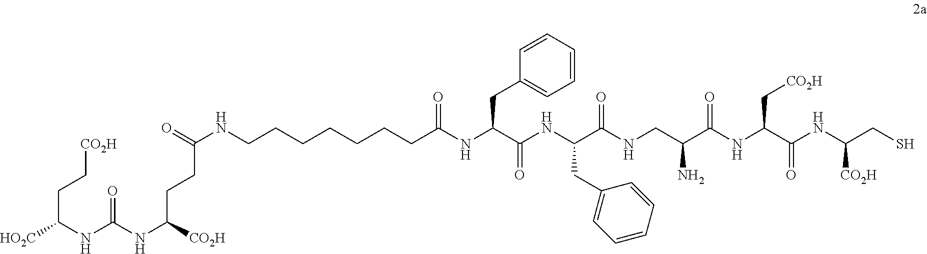 [SMILES: N[C@@H](CNC(=O)[C@H](Cc1ccccc1)NC(=O)[C@H](Cc1ccccc1)NC(=O)CCCCCCCNC(=O)CC[C@H](NC(=O)N[C@@H](CCC(=O)O)C(=O)O)C(=O)O)C(=O)N[C@@H](CC(=O)O)C(=O)N[C@@H](CS)C(=O)O]

or a pharmaceutically acceptable salt thereof, wherein a radionuclide is bound to the conjugate.

In some aspects of these embodiments, the PSMA ligand-imaging conjugate is of the formula 3a

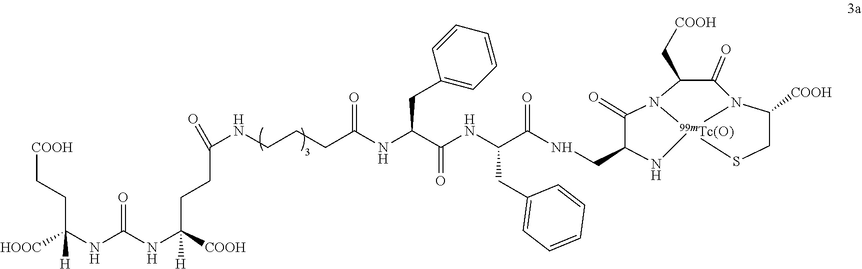

or a pharmaceutically acceptable salt thereof.

In some aspects of these embodiments, determining as described herein comprises administering to the patient a PSMA ligand-imaging conjugate of the formula 4

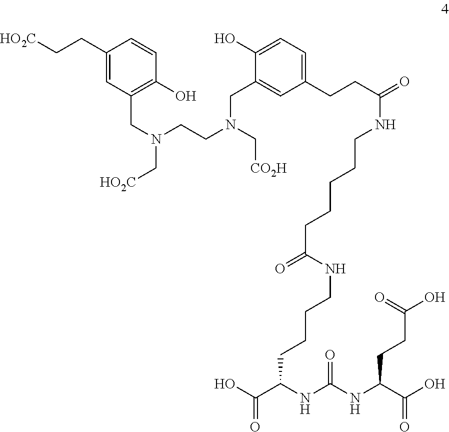 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

or a pharmaceutically acceptable salt thereof, wherein a radionuclide is bound to the conjugate. In some aspects of these embodiments, the radionuclide is ⁶⁷Ga or ⁶⁸Ga.

In some aspects of these embodiments, determining as described herein comprises detecting the compound of the formula I-Lu or Ia-Lu administered for the purpose of treating.

In some embodiments, the shielding agent useful in connection with the present disclosure, and the various methods described herein, can be a compound selected from the group consisting of

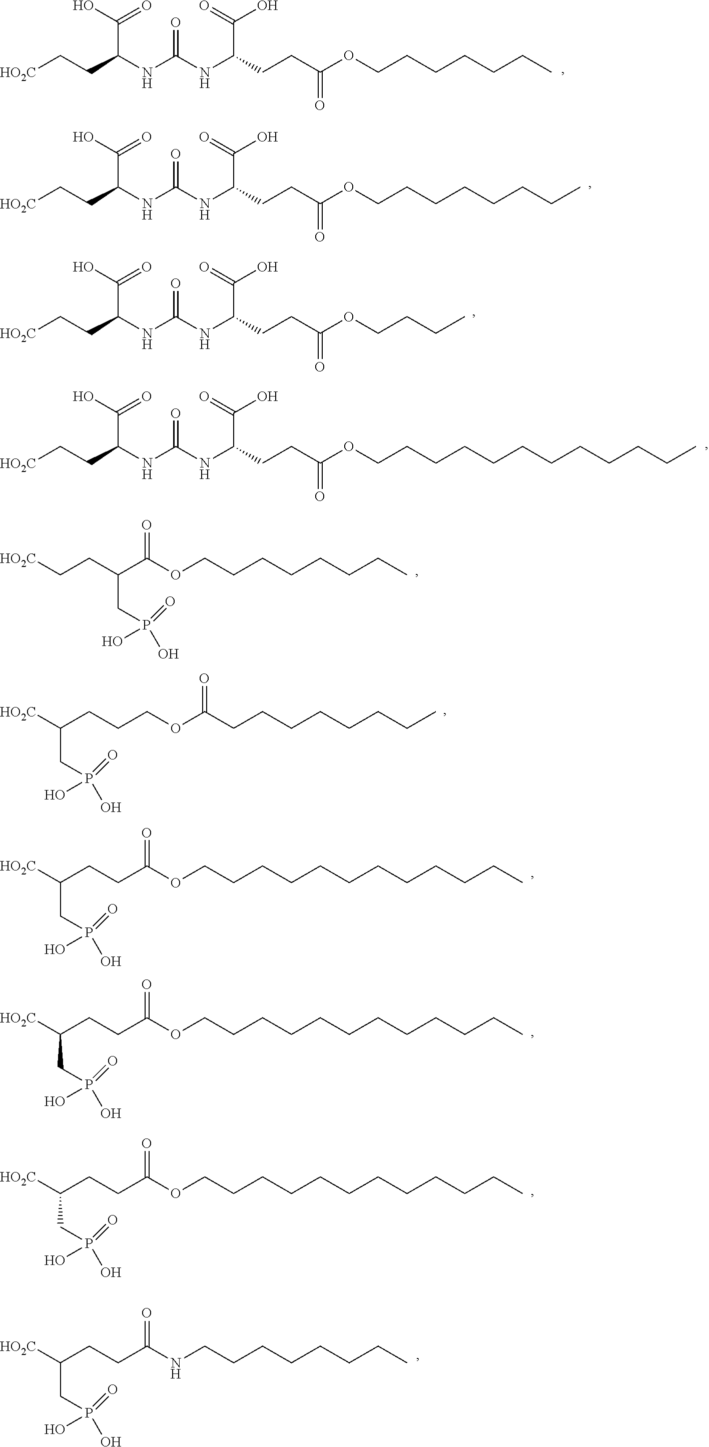

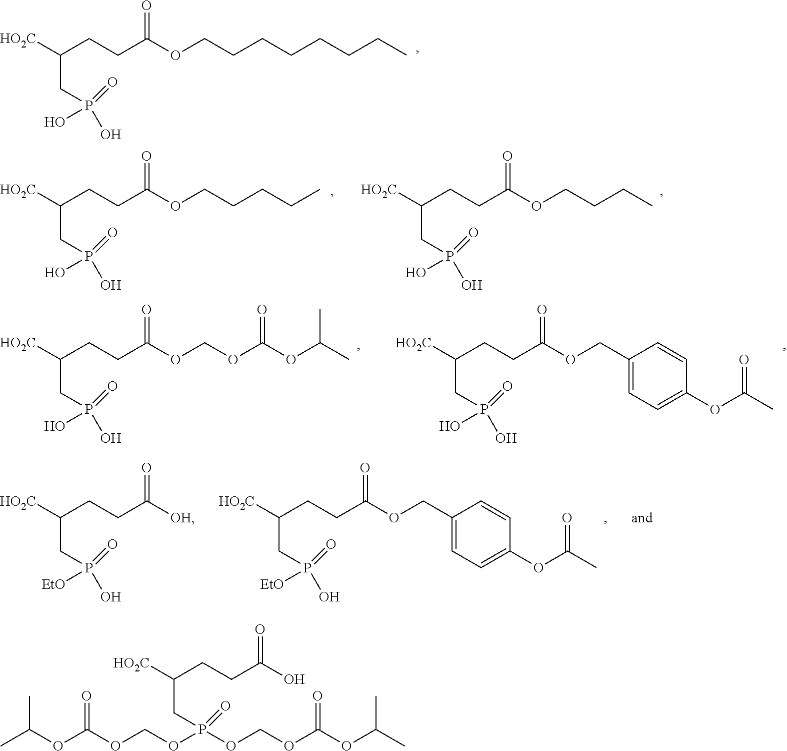

In some embodiments, the present disclosure provides a compound selected from the group consisting of

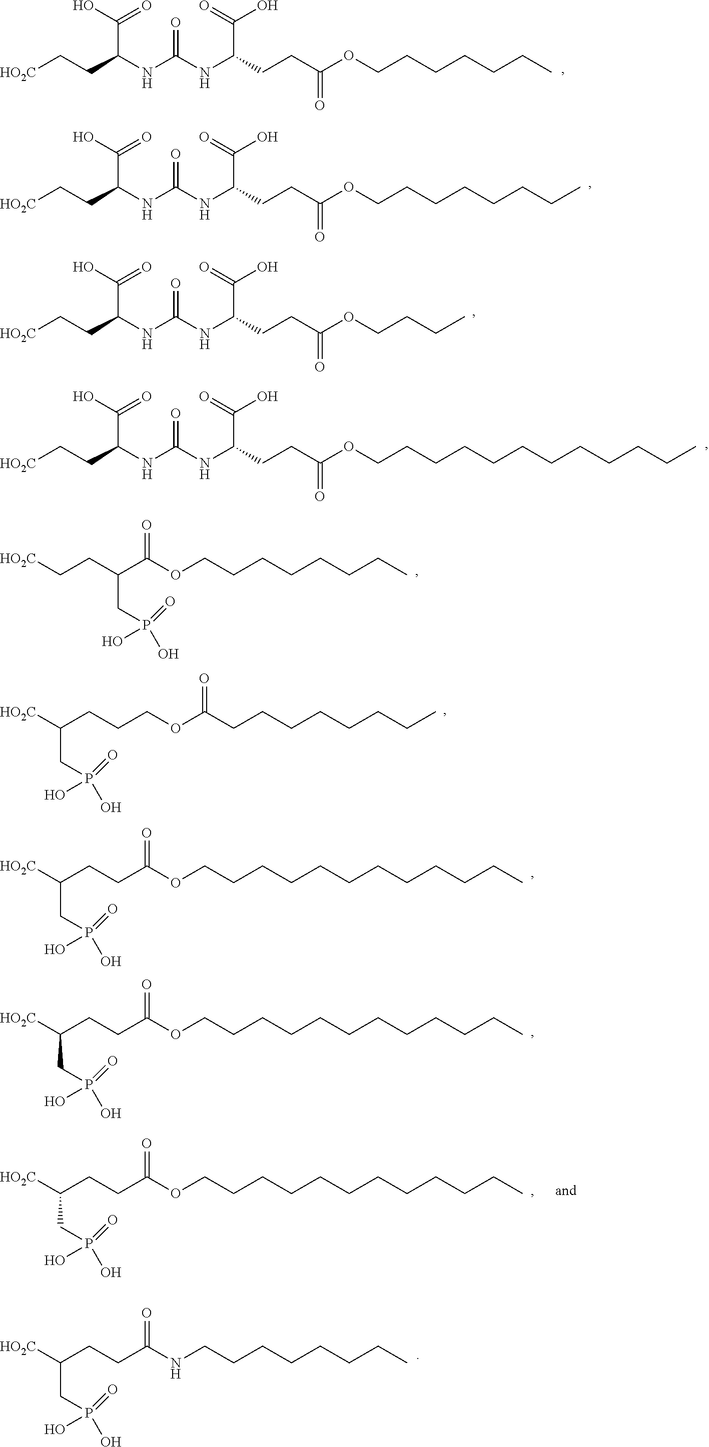

Certain embodiments are further described by the following enumerated clauses:

1. A compound selected from the group consisting of

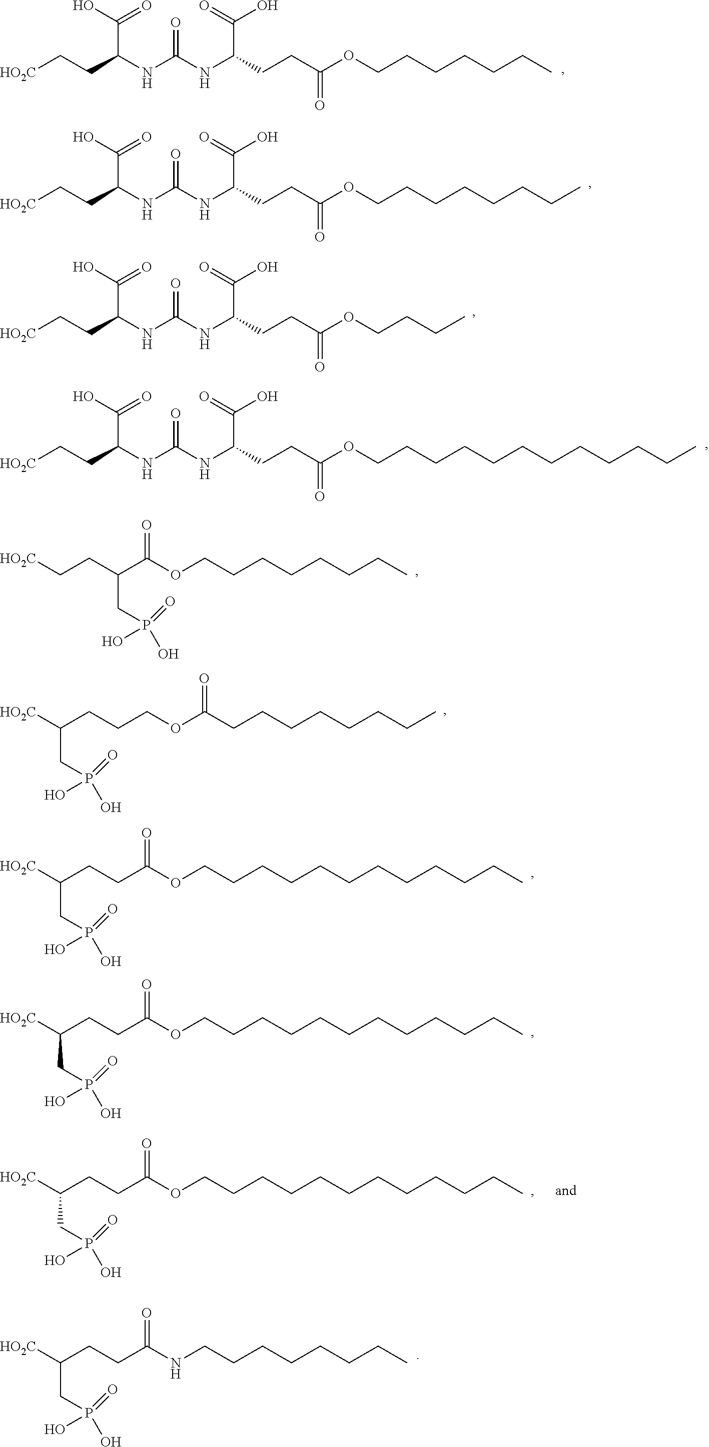

2. A method for treating a cancer in a patient comprising administering a therapeutically effective amount of a radiolabeled therapeutic in combination with a an effective amount of a shielding agent.

3. The method of clause 2, wherein the radiolabeled therapeutic is Compound Ia-Lu or Ia-Ac.

4. The method of clause 2 or 3, wherein the cancer is a prostate cancer.

5. The method of any one of clauses 2 to 4, wherein the cancer is a metastatic prostate cancer.

6. The method of any one of clauses 2 to 4, wherein the cancer is a metastatic castration-resistant prostate cancer.

7. The method of any one of clauses 2 to 6, wherein the shielding agent is selected from the group consisting of

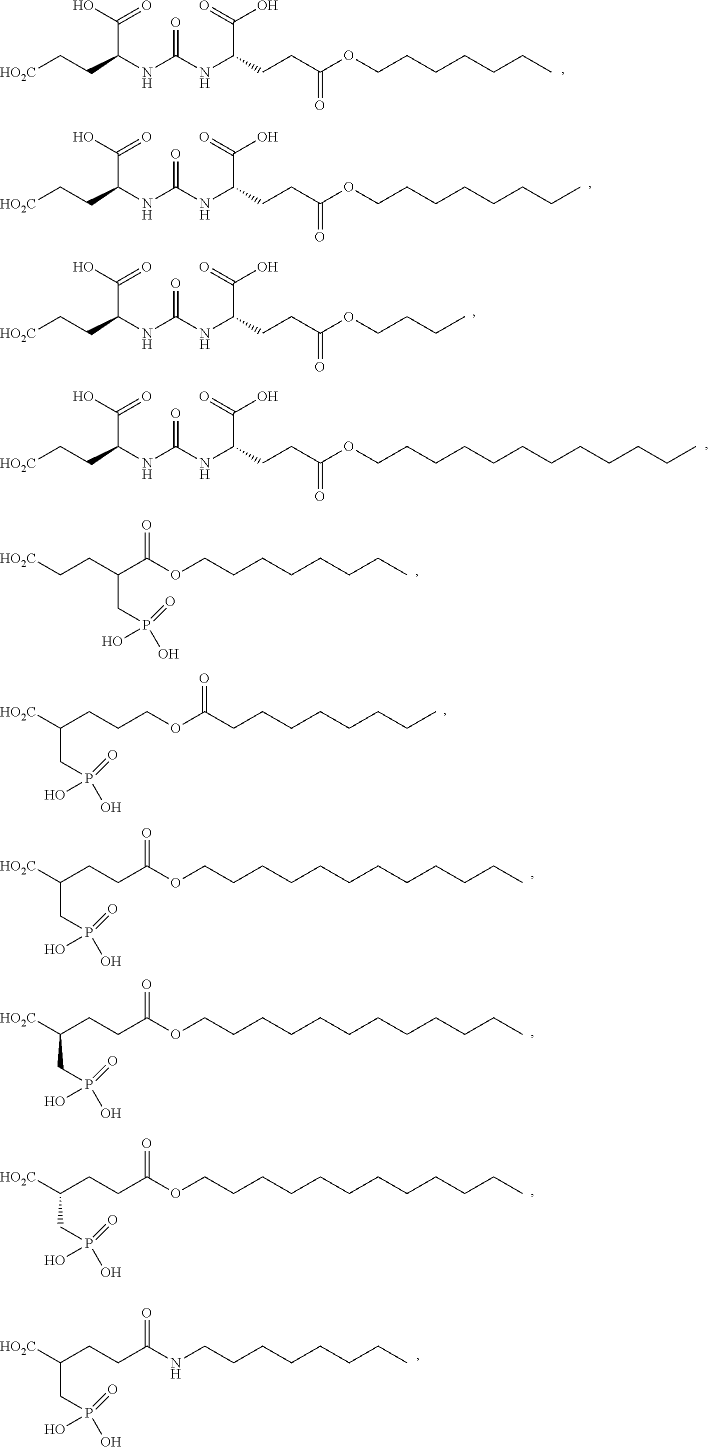

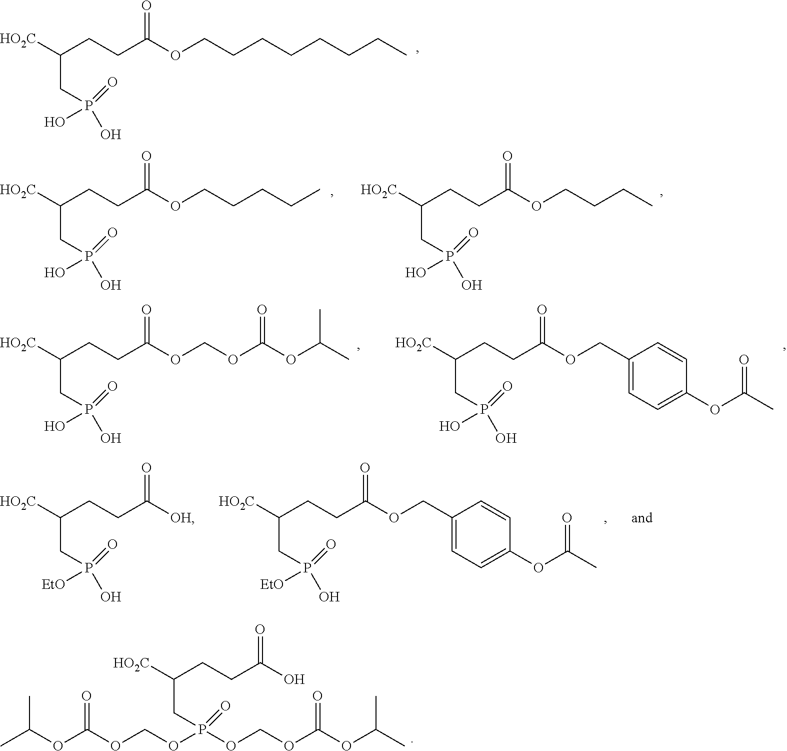

8. The method of any one of clauses 2 to 6, wherein the shielding agent is selected from the group consisting of

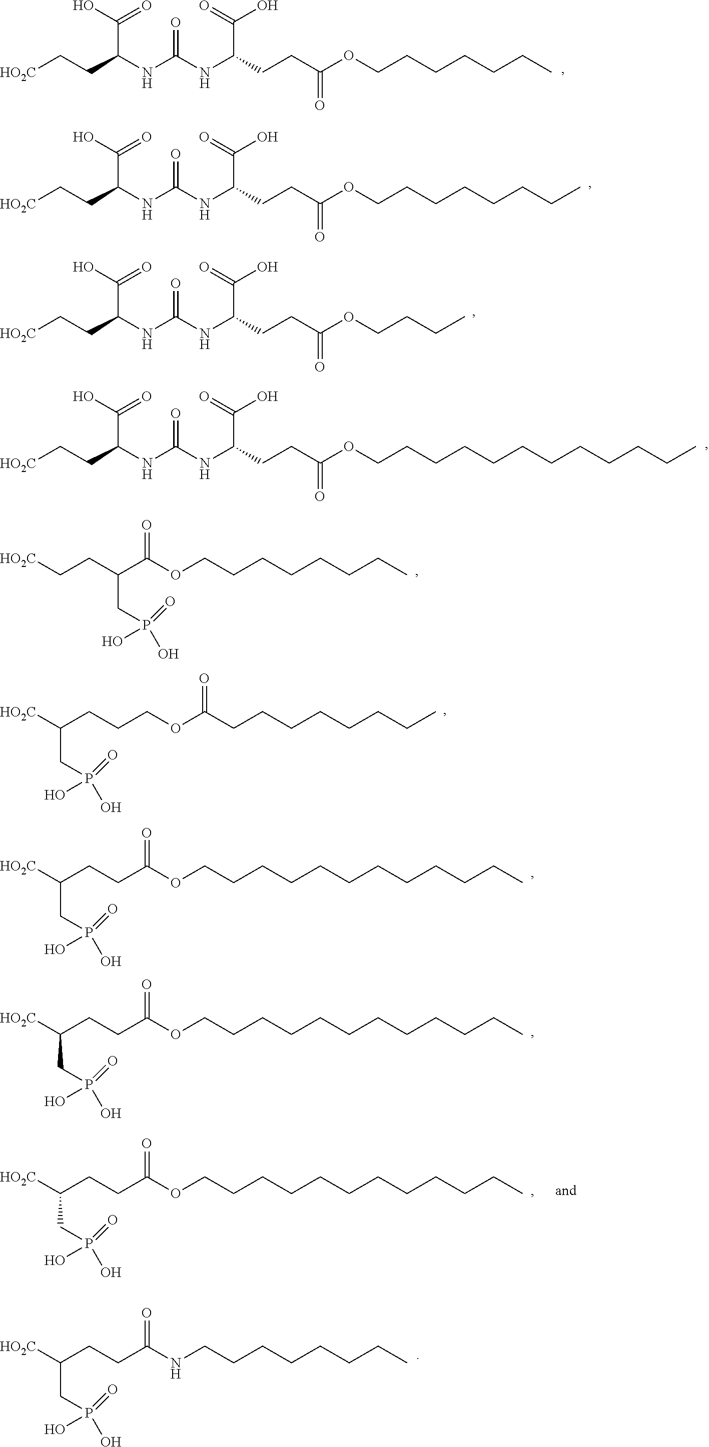

9. A compound selected from the group consisting of

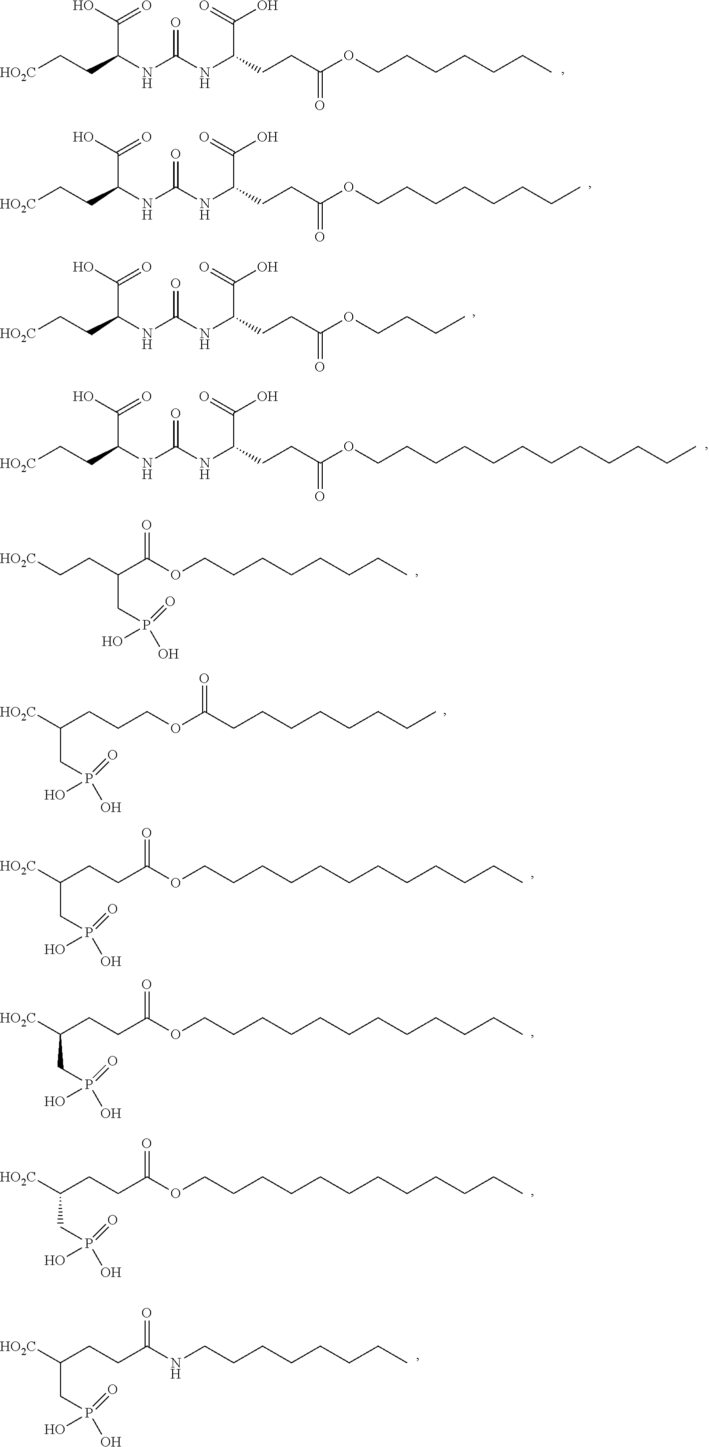

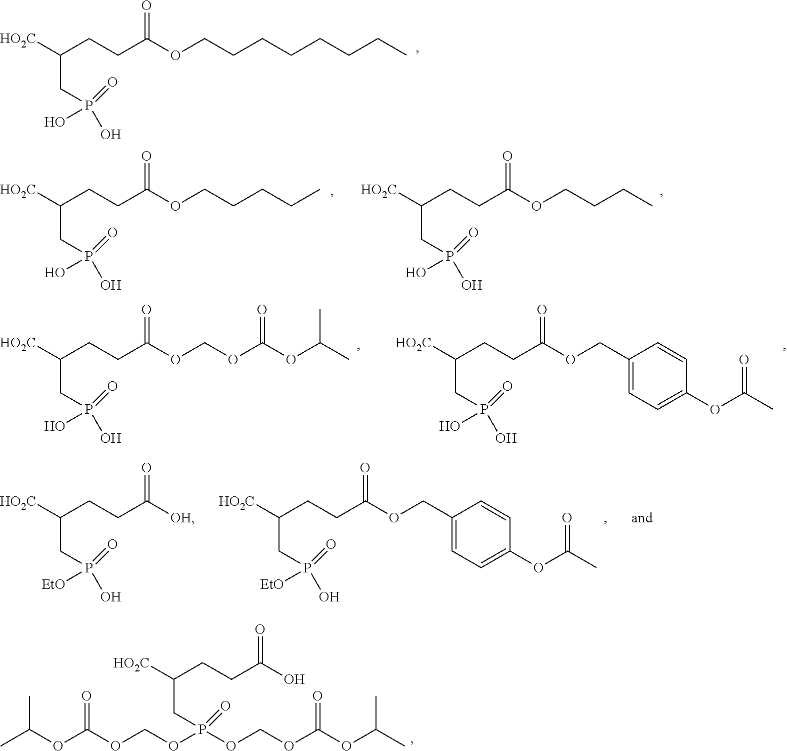

for treating cancer in a patient in need of such treatment in combination with a therapeutically effective amount of a radiolabeled therapeutic.

10. The compound of clause 9, wherein the radiolabeled therapeutic is Compound Ia-Lu or Ia-Ac.

11. The compound of clause 9 or 10, wherein the cancer is a prostate cancer.

12. The compound of any one of clauses 9 to 11, wherein the cancer is a metastatic prostate cancer.

13. The compound of any one of clauses 9 to 11, wherein the cancer is a metastatic castration-resistant prostate cancer.

14. Use of compound selected from the group consisting of

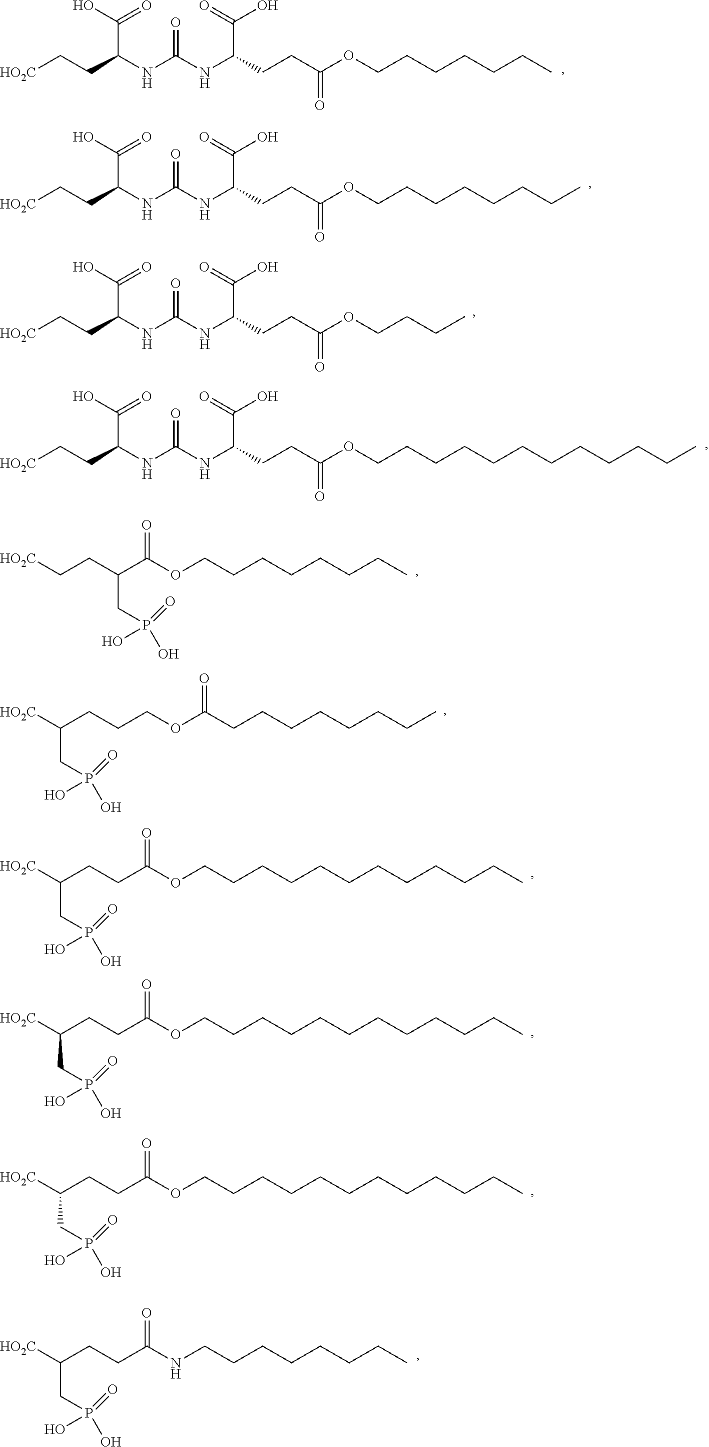

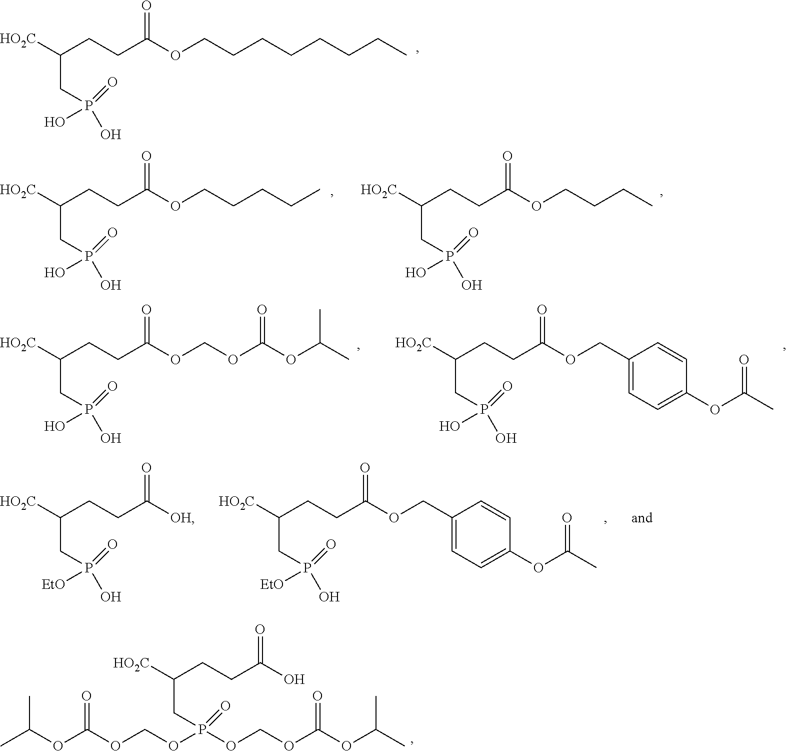

in the manufacture of a medicament for treating cancer in a patient in combination with a therapeutically effective amount of a radiolabeled therapeutic.

15. The use of clause 14, wherein the radiolabeled therapeutic is Compound Ia-Lu or Ia-Ac.

16. The use of clause 14 or 15, wherein the cancer is a prostate cancer.

17. The use of any one of clauses 14 to 16, wherein the cancer is a metastatic prostate cancer.

18. The use of any one of clauses 14 to 16, wherein the cancer is a metastatic castration-resistant prostate cancer. 19. A method for imaging a cancer in a patient comprising administering an effective amount of an imaging conjugate in combination with an effective amount of a shielding agent.

20. The method of clause 19, wherein the imaging is ^{99m}Tc labelled imaging conjugate 3a or ⁶⁷Ga or⁶⁸Ga labelled imaging conjugate 4.

21. The method of clause 19 or 20, wherein the cancer is a prostate cancer.

22. The method of any one of clauses 19 to 21, wherein the cancer is a metastatic prostate cancer.

23. The method of any one of clauses 19 to 22, wherein the cancer is a metastatic castration-resistant prostate cancer. 24. The method of any one of clauses 19 to 23, wherein the shielding agent is selected from the group consisting of

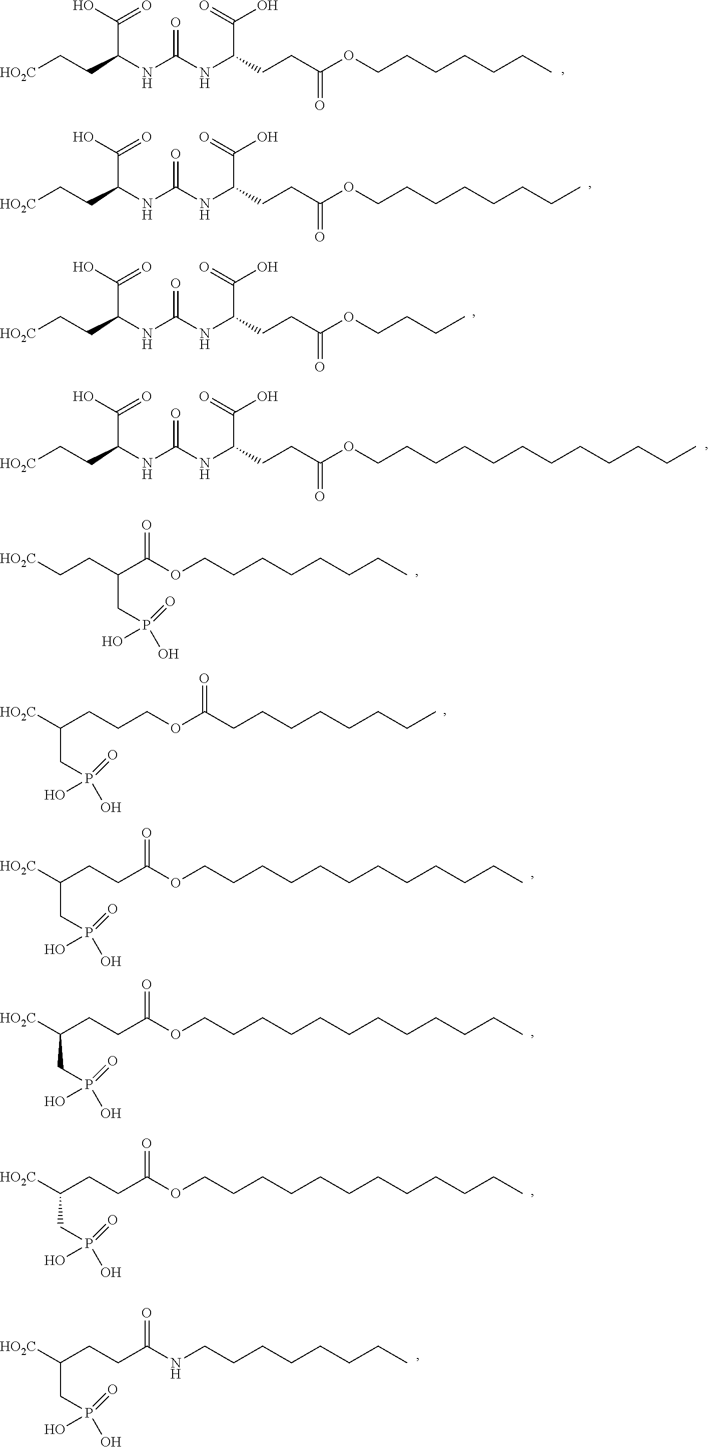

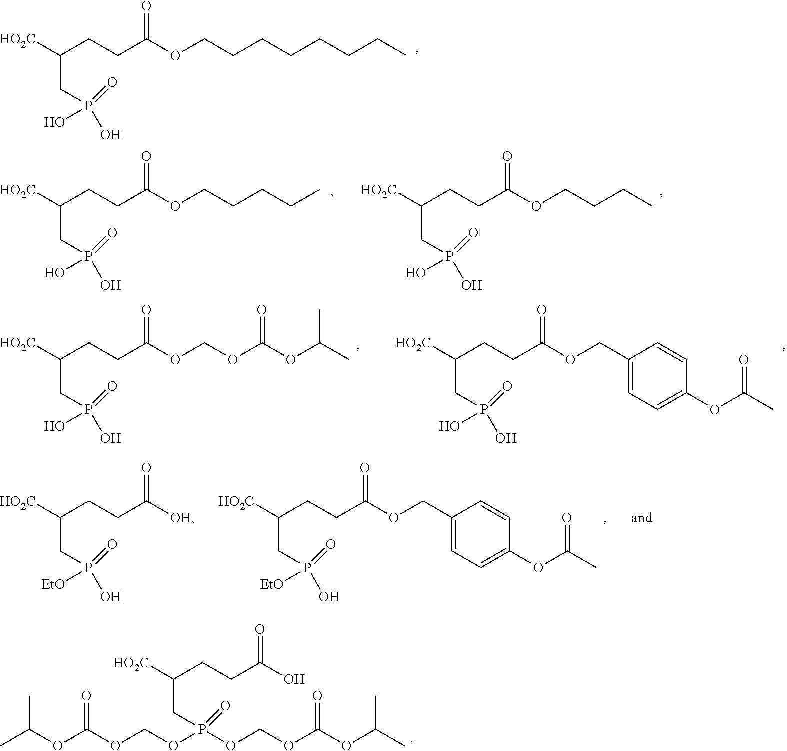

25. The method of any one of clauses 19 to 23, wherein the shielding agent is selected from the group consisting of

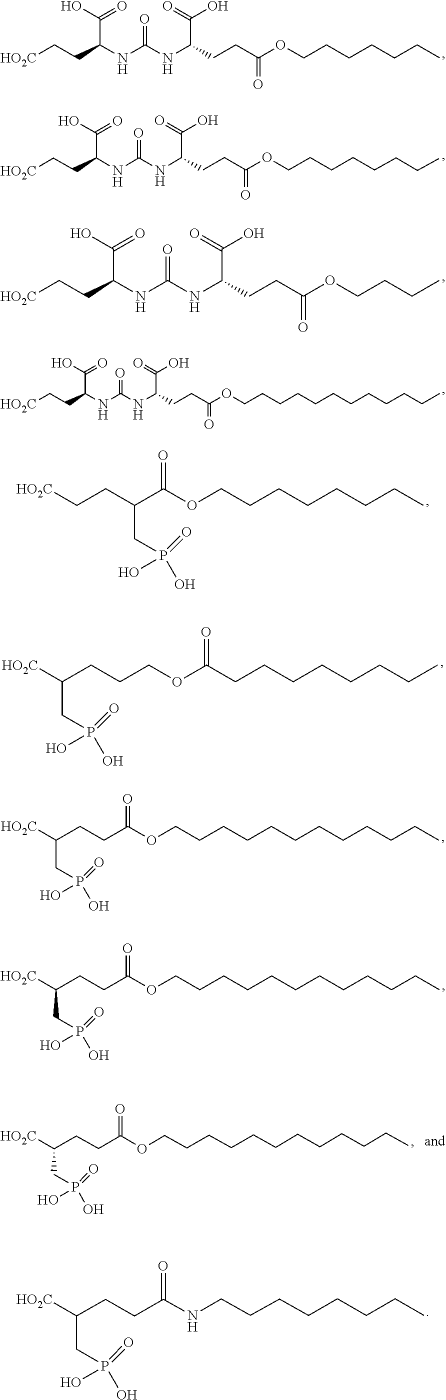

26. A compound selected from the group consisting of

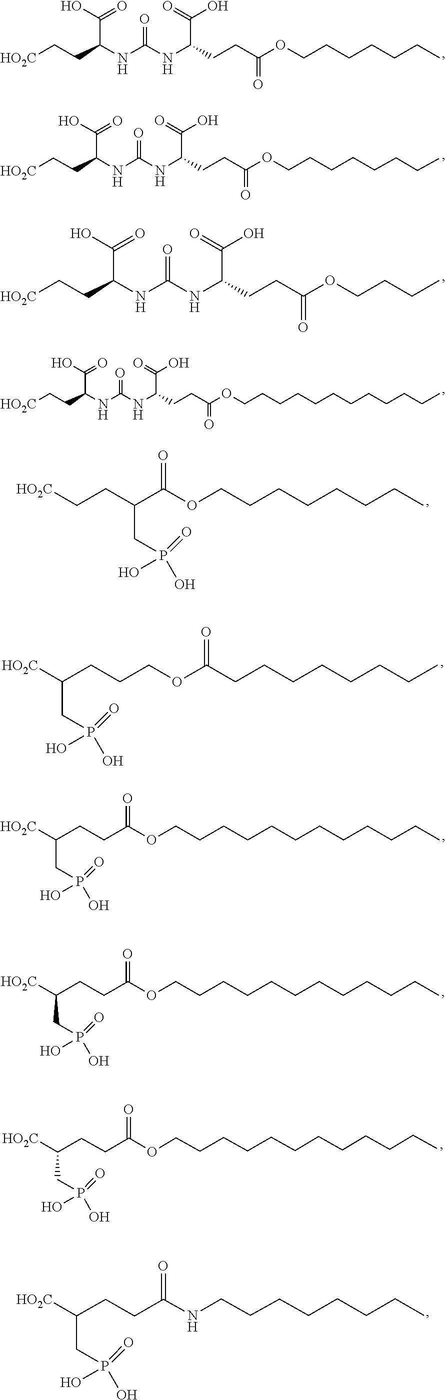

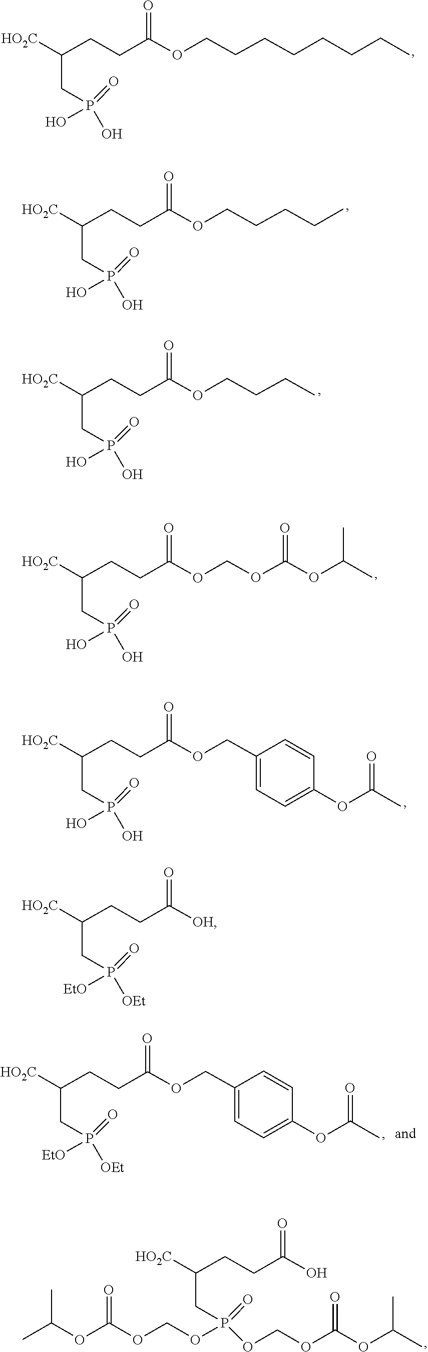

for imaging cancer in a patient in need of such treatment in combination with an effective amount of an imaging conjugate.

27. The compound of clause 26, wherein the imaging conjugate is ^{99m}Tc labelled imaging conjugate 3a or ⁶⁷Ga or ⁶⁸Ga labelled imaging conjugate 4.

28. The compound of clause 26 or 27, wherein the cancer is a prostate cancer.

29. The compound of any one of clauses 26 to 28, wherein the cancer is a metastatic prostate cancer.

30. The compound of any one of clauses 26 to 28, wherein the cancer is a metastatic castration-resistant prostate cancer. 31. Use of compound selected from the group consisting of

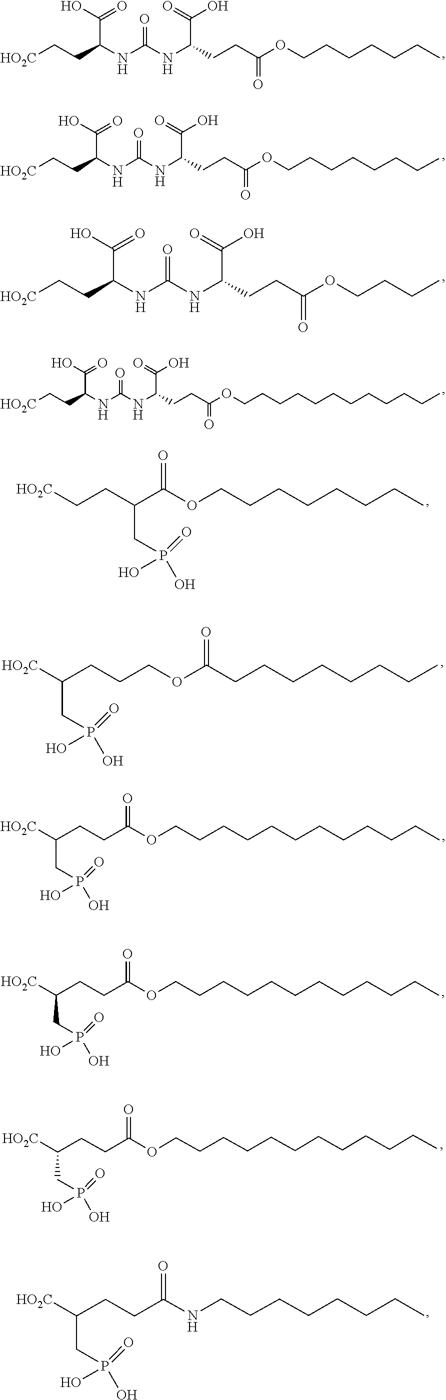

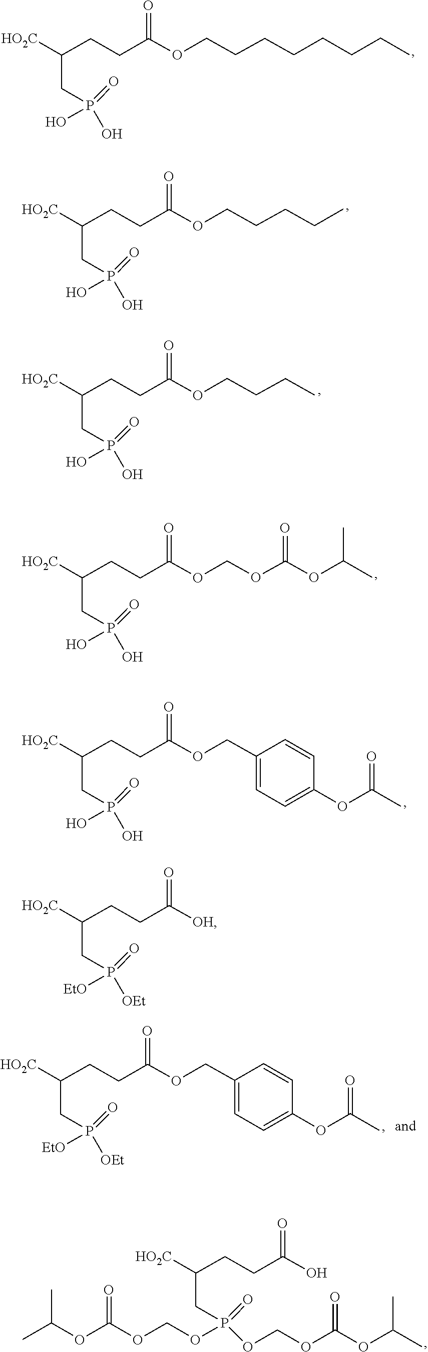

in the manufacture of a medicament for imaging cancer in a patient in combination with an effective amount of n imaging conjugate.

32. The use of clause 31, wherein the imaging conjugate is ^{99m}Tc labelled imaging conjugate 3a or ⁶⁷Ga or ⁶⁸Ga labelled imaging conjugate 4.

33. The use of clause 31 or 32, wherein the cancer is a prostate cancer.

34. The use of any one of clauses 31 to 33, wherein the cancer is a metastatic prostate cancer.

35. The use of any one of clauses 31 to 34, wherein the cancer is a metastatic castration-resistant prostate cancer.

### BRIEF DESCRIPTION OF THE DRAWINGS

FIG. 1 is a chart showing the biodistribution in nude mice at 30 nmol/kg of ^{99m}TC imaging conjugate 3a with or without co-administration of 0.5 μmol/kg of a shielding agent. In the graph for each tissue ^{99m}TC imaging conjugate 3a (far left bar), ^{99m}TC imaging conjugate 3a+compound 1a (second from left bar), ^{99m}TC imaging conjugate 3a+compound 1b (third from left bar), ^{99m}TC imaging conjugate 3a+compound 1d (third from right bar), ^{99m}TC imaging conjugate 3a+compound 1c (second from right bar), ^{99m}TC imaging conjugate 3a+compound 1e (far right bar).

FIG. 2 is a chart showing the biodistribution in nude mice at 30 nmol/kg of ^{99m}TC imaging conjugate 3a with or without co-administration of 10 μmol/kg of a shielding agent. In the graph for each tissue ^{99m}TC imaging conjugate 3a (far left bar), ^{99m}TC imaging conjugate 3a+compound 1j (second from left bar), ^{99m}TC imaging conjugate 3a+compound 1k (middle bar), ^{99m}TC imaging conjugate 3a+compound 1f (second from right bar), ^{99m}TC imaging conjugate 3a+competitor PMPA (far right bar).

FIG. 3 is a chart showing the biodistribution in nude mice at 30 nmol/kg of ^{99m}TC imaging conjugate 3a with or without co-administration of 0.5 μmol/kg of a shielding agent. In the graph for each tissue ^{99m}TC imaging conjugate 3a (far left bar), ^{99m}TC imaging conjugate 3a+compound JHU-2545 (E1) (second from left bar), ^{99m}TC imaging conjugate 3a+compound JHU-2545 (E2) (third from left bar), ^{99m}TC imaging conjugate 3a+compound 11 (E1) (third from right bar), ^{99m}TC imaging conjugate 3a+compound 11 (E2) (second from right bar), ^{99m}TC imaging conjugate 3a+compound 1f at 1 μmol/kg (far right bar).

FIG. 4 is a chart showing the biodistribution in nude mice at 30 nmol/kg of ^{99m}TC imaging conjugate 3a with or without co-administration of 1 μmol/kg of a shielding agent. In the graph for each tissue ^{99m}TC imaging conjugate 3a (left bar), ^{99m}TC imaging conjugate 3a+compound 1a (right bar).

FIG. 5 is a chart showing the biodistribution in nude mice at 30 nmol/kg of ^{99m}TC imaging conjugate 3a with or without co-administration of 1 μmol/kg of a shielding agent. In the graph for each tissue ^{99m}TC imaging conjugate 3a (left bar), ^{99m}TC imaging conjugate 3a +compound 1d (right bar).

FIG. 6 is a chart showing the biodistribution in nude mice at 30 nmol/kg of ⁶⁷Ga-Ia with or without co-administration of 10 μmol/kg of a shielding agent. In the graph for each tissue ⁶⁷Ga-Ia (far left bar), ⁶⁷Ga-Ia+compound 1i (second from left bar), ⁶⁷Ga-Ia+compound 1g (middle bar), ⁶⁷Ga-Ia+compound 1h (second from right bar), ⁶⁷Ga-Ia+competitor PMPA (far right bar).

FIG. 7 is a chart showing the biodistribution between tumors and kidney (tumor/kidney or T/K ratio) of ^{99m}TC imaging conjugate 3a in nude mice at various pre-treatment doses (μmol/kg) of shielding agents of the present disclosure. (▴)^{99m}TC imaging conjugate 3a+compound 1a; (▪) ^{99m}TC imaging conjugate 3a+compound 1d; (▾) ^{99m}TC imaging conjugate 3a+compound 1n.

FIG. 8 is a chart showing the biodistribution at 4 hours in nude mice bearing 22RV1 tumors at 30 nmol/kg of ^{99m}TC imaging conjugate 3a with co-administration of 1 μmol/kg of a shielding agent. The chart shows that the S-enantiomer of shielding agent 1d is more active than the R-enantiomer, and provided an enhanced ^{99m}TC imaging conjugate 3a tumor to kidney ratio. In the graph for each tissue ^{99m}TC imaging conjugate 3a+compound 1d (S-enantiomer) (left bar), ^{99m}TC imaging conjugate 3a+compound 1d (R-enantiomer) (right bar).

FIG. 9 is a chart showing the biodistribution at 4 hours in nude mice bearing LNCaP tumors at 30 nmol/kg of Ia-Lu with or without co-administration of 1 μmol/kg of a shielding agent. The chart shows that both shielding agent 1d (S-enantiomer) and shielding agent 1m provided an enhanced Ia-Lu tumor to kidney ratio. In the graph for each tissue Ia-Lu alone (left bar), Ia-Lu+compound 1m (middle bar), Ia-Lu+compound 1d (S-enantiomer) (right bar).

### DEFINITIONS

As used herein, “functionally active PSMA” means a cell surface membrane-bound glycoprotein that binds to a PSMA ligand. It will be appreciated that PSMA ligands are well known to those skilled in the art such as those described in US patent publication no. US 2010/0324008 A1, incorporated herein by reference.

As used herein, “clinical benefit” means a response of a patient to treatment with a combination of Compounds I-Lu or Ia-Lu, and I-Ac or Ia-Ac, where the response includes overall survival of the patient, ability to receive four or more cycles of therapy (e.g., four weeks of therapy) with Compounds I-Lu or Ia-Lu, and I-Ac or Ia-Ac, inhibition of tumor growth, stable disease, a partial response, and/or a complete response, among other clinical benefits defined by the Food and Drug Administration in the United States of America.

As used herein, “inhibition of tumor growth” means reduction in tumor size, complete disappearance of a tumor, or growth of a patient tumor of less than 30% over the course of therapy with a combination of Compounds I-Lu or Ia-Lu, and I-Ac or Ia-Ac.

As used herein, “stable disease” means no material progression of disease in a patient over the course of therapy with a combination of Compounds I-Lu or Ia-Lu, and I-Ac or Ia-Ac.

As used herein, “a partial response” means a decrease in tumor size of 30% or greater in a patient treated with a combination of Compounds I-Lu or Ia-Lu, and I-Ac or Ia-Ac.

As used herein, “a complete response” means the disappearance of detectable disease in a patient treated with a combination of Compounds I-Lu or Ia-Lu, and I-Ac or Ia-Ac.

As used herein, “prior treatment” means the patient has been treated with at least one prior treatment known in the art. It will be appreciated that a prior treatment can be any treatment known to those of skill in the art, including, but not limited, chemotherapeutic agent, surgery, radiation therapy, immunotherapy, photodynamic therapy, stem cell therapy, hyperthermia, and the like. Prior treatments can include systemic treatments including, but not limited to treatment with abiraterone, orteronel, galeterone, seviteronel, apalutamide, enzalutamide, palifosfamide, 5-fluorouracil, capecitabine, pemetrexed, cisplatin, carboplatin, gemcitabine, paclitaxel, vinorelbine, eribulin, docetaxel, cyclophosphamide, doxorubicin, regorafinib, and combinations thereof.

As used herein, the term “alkyl” includes a chain of carbon atoms, which is optionally branched. It will be further understood that in certain embodiments, alkyl is advantageously of limited length, including C₁-C₂₄, C₁-C₈, C₁-C₆, and C₁-C₄. Illustratively, such particularly limited length alkyl groups, including C₁-C₈, C₁-C₆, and C₁-C₄ may be referred to as lower alkyl. It is appreciated herein that shorter alkyl, alkenyl, and/or alkynyl groups may add less lipophilicity to the compound and accordingly will have different pharmacokinetic behavior. In some embodiments, it will be understood, in each case, that the recitation of alkyl refers to alkyl as defined herein, and optionally lower alkyl. Illustrative alkyl groups include, but not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like. As used herein, a “carboxyalkyl” group includes a combination of an “alkyl” group as described herein with a “carboxy” group. As used herein, a “hydroxyalkyl” group includes a combination of an “alkyl” group as described herein with a “hydroxy” group. As used herein, a “aminoalkyl” group includes a combination of an “alkyl” group as described herein with a “amino” group.

As used herein, the term “heteroalkyl” includes a chain of atoms that includes both carbon and at least one heteroatom, and is optionally branched. Illustrative heteroatoms include nitrogen, oxygen, and sulfur. In certain variations, illustrative heteroatoms also include phosphorus, and selenium.

As used herein, the term “aryl” includes monocyclic and polycyclic aromatic carbocyclic groups having from 6 to 14 ring carbon atoms, each of which may be optionally substituted. Illustrative aromatic carbocyclic groups described herein include, but are not limited to, phenyl, naphthyl, and the like. As used herein, the term “heteroaryl” includes aromatic heterocyclic groups, having from 5 to 10 ring atoms, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like. As used herein, the term “heteroarylalkyl” includes a combination of an “alkyl” group as described herein with a “heteroaryl” group described herein. As used herein, the term “arylalkyl” includes a combination of an “alkyl” group as described herein with a “aryl” group described herein, for example a benzyl group.

The term “optionally substituted” as used herein includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

As used herein, the term “administering” as used herein includes all means of introducing Compounds I-Lu, Ia-Lu, I-Ac, or Ia-Ac and/or a PSMA ligand-imaging conjugate as described herein to the patient, including, but not limited to, oral (po), intravenous (iv), intramuscular (im), subcutaneous (sc), transdermal, inhalation, buccal, ocular, sublingual, vaginal, rectal, and the like. A combination of Compounds I-Lu or Ia-Lu, and I-Ac or Ia-Ac and/or a PSMA ligand-imaging conjugate as described herein may be administered in unit dosage forms and/or formulations containing conventional nontoxic pharmaceutically-acceptable carriers, adjuvants, and vehicles.

As used herein, “becquerel” means a SI derived unit of radioactivity as it is commonly understood by one of skill in the art. One becquerel is defined as the activity of a quantity of radioactive material in which one nucleus decays per second. A becquerel is therefore equivalent to an inverse second, s-1. The becquerel is known to one of skill in the art as the successor of the curie (Ci), an older, non-SI unit of radioactivity based on the activity of 1 gram of radium-226. The curie is defined as 3.7. 1010 s-1, or 37 GBq.

As used herein, “curie” or “Ci” means a unit of radioactivity named after the French physicist and chemist Marie Curie as commonly understood by one of skill in the art. The prefixes milli and micro are from the metric system and represent 0.001 and 0.000001, respectively. So, a millicurie (mCi) is 0.001 curie. A microcurie (μCi) is 0.000001 curie.

### DETAILED DESCRIPTION

The embodiments of the numbered clauses provided in the summary above, or any combination thereof, are contemplated for combination with any of the embodiments described in the Detailed Description section of this patent application.

In one embodiment, the methods described herein can be used for both human clinical medicine and veterinary applications. Thus, a “patient” can be administered Compound I-Lu, Ia-Lu, I-Ac, or Ia-Ac and/or PSMA ligand-imaging conjugates described herein in combination with a shielding agent as described herein, and can be human or, in the case of veterinary applications, can be a laboratory, agricultural, domestic, or wild animal. In one aspect, the patient can be a human, a laboratory animal such as a rodent (e.g., mice, rats, hamsters, etc.), a rabbit, a monkey, a chimpanzee, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

In various embodiments, the cancers described herein can be a cancer cell population that is tumorigenic, including benign tumors and malignant tumors, or the cancer can be non-tumorigenic. The cancer can arise spontaneously or by such processes as mutations present in the germline of the patient or somatic mutations, or the cancer can be chemically-, virally-, or radiation-induced. Cancers applicable to the present disclosure described herein include, but are not limited to, a glioma, a carcinoma, a sarcoma, a lymphoma, a melanoma, a mesothelioma, a nasopharyngeal carcinoma, a leukemia, an adenocarcinoma, and a myeloma.

In some aspects the cancers can be lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, metastatic breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, glioma, brain stem glioma, pituitary adenoma, and adenocarcinoma of the gastroesophageal junction.

Compound Ia has the formula

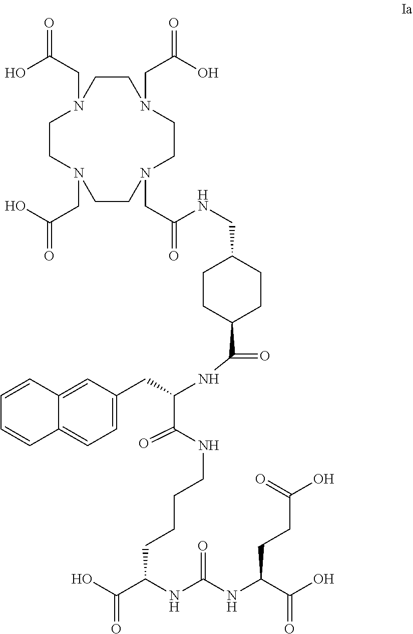 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O]

wherein ¹⁷⁷Lu is complexed to the compound in Ia-Lu, and ²²⁵Ac is complexed to the compound in Ia-Ac.

In other embodiments, any of a variety of PSMA ligand-imaging conjugates detectable by PET imaging, SPECT imaging, and the like can be used. The exact manner of imaging is not limited to the imaging agents described herein. Collectively, the PSMA ligand-imaging conjugates useful for imaging described herein, including those described by formulas and the agents useful for PET imaging, SPECT imaging, etc. are referred to as “PSMA ligand-imaging conjugates.”

Shielding agents useful in connection with the present disclosure can be any shielding agent capable of blocking the off target binding of a radiolabeled compound as described herein to PSMA. Suitable shielding agents include, but are not limited to those described in US. patent publication US 2017/0226141, Majer, P. et al., “Discovery of Orally Available Prodrugs of the Glutamate Carboxypeptidase II (GCPII) Inhibitor 2-Phosphonomethylpentanedoic Acid (2-PMPA)” J. Med. Chem., 59, 2810-2819 (2016), and Nedelcovych M. et al , “Enhanced Brain Delivery of 2-(Phosphonomethyl)pentanedioic Acid Following Intranasal Administration of Its γ-Substituted Ester” Mol. Pharmaceutics, 14, 3248-3257 (2017), the disclosures of which are incorporated by reference. Suitable examples of shielding agents include, but are not limited to those shown in Table 1.

| CPD | Structure |
|---|---|
| 1a | 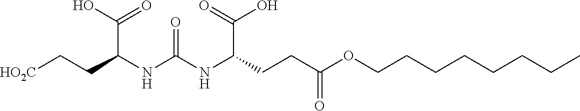 [SMILES: CCCCCCCCOC(=O)CC[C@H](NC(=O)N[C@@H](CCC(=O)O)C(=O)O)C(=O)O] |
| 1b | 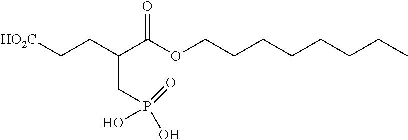 [SMILES: CCCCCCCCOC(=O)C(CCC(=O)O)CP(=O)(O)O] |
| 1c | 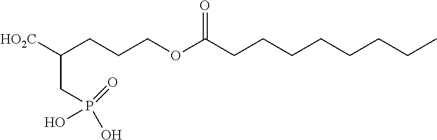 [SMILES: CCCCCCCCC(=O)OCCCC(CP(=O)(O)O)C(=O)O] |
| 1d | 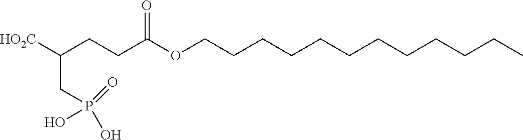 [SMILES: CCCCCCCCCCCCOC(=O)CCC(CP(=O)(O)O)C(=O)O] |
| 1d(R) | 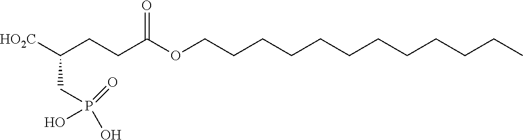 [SMILES: CCCCCCCCCCCCOC(=O)CC[C@H](CP(=O)(O)O)C(=O)O] |
| 1d(S) | 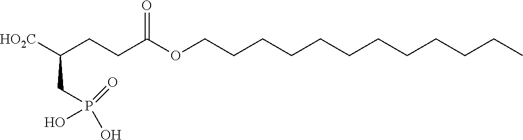 [SMILES: CCCCCCCCCCCCOC(=O)CC[C@@H](CP(=O)(O)O)C(=O)O] |
| 1e | 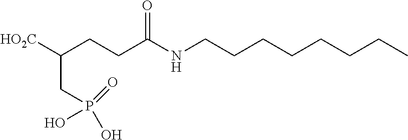 [SMILES: CCCCCCCCNC(=O)CCC(CP(=O)(O)O)C(=O)O] |
| 1f | 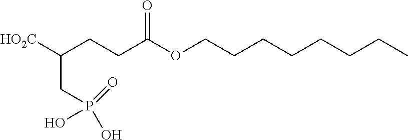 [SMILES: CCCCCCCCOC(=O)CCC(CP(=O)(O)O)C(=O)O] |
| 1g | 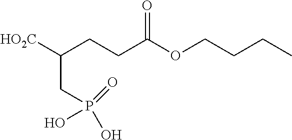 [SMILES: CCCCOC(=O)CCC(CP(=O)(O)O)C(=O)O] |
| 1h | 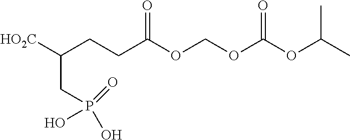 [SMILES: CC(C)OC(=O)OCOC(=O)CCC(CP(=O)(O)O)C(=O)O] |
| 1i | 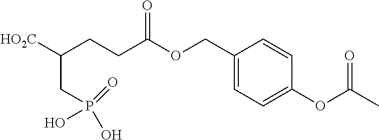 [SMILES: CC(=O)Oc1ccc(COC(=O)CCC(CP(=O)(O)O)C(=O)O)cc1] |
| 1j | 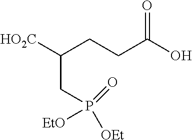 [SMILES: CCOP(=O)(CC(CCC(=O)O)C(=O)O)OCC] |
| 1k | 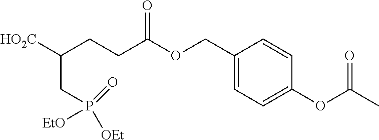 [SMILES: CCOP(=O)(CC(CCC(=O)OCc1ccc(OC(C)=O)cc1)C(=O)O)OCC] |
| 1l | 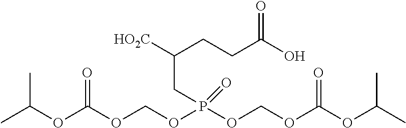 [SMILES: CC(C)OC(=O)OCOP(=O)(CC(CCC(=O)O)C(=O)O)OCOC(=O)OC(C)C] |
| 1m | 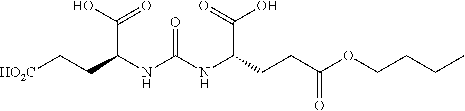 [SMILES: CCCCOC(=O)CC[C@H](NC(=O)N[C@@H](CCC(=O)O)C(=O)O)C(=O)O] |
| 1n | 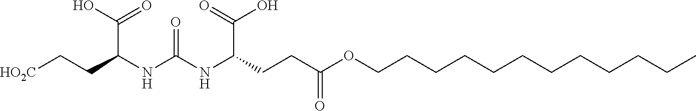 [SMILES: CCCCCCCCCCCCOC(=O)CC[C@H](NC(=O)N[C@@H](CCC(=O)O)C(=O)O)C(=O)O] |

In one embodiment, the Compounds I-Lu, Ia-Lu, I-Ac, or Ia-Ac and/or PSMA ligand-imaging conjugates described herein bind to PSMA expressed on cancer cells. In some embodiments, the shielding agents described herein bind to PSMA. In one illustrative aspect, the Compounds I-Lu, Ia-Lu, I-Ac, or Ia-Ac and/or PSMA ligand-imaging conjugates are capable of differentially binding to PSMA on cancer cells compared to normal cells due to preferential expression (or over-expression) of PSMA on the cancer cells. In some embodiments, the shielding agents described herein are capable of blocking the off target binding of Compounds I-Lu, Ia-Lu, I-Ac, or Ia-Ac and/or PSMA ligand-imaging conjugates to PSMA expressed on, for example, liver cells.

In some embodiments, Compounds I-Lu, Ia-Lu, I-Ac, or Ia-Ac and/or PSMA ligand-imaging conjugates described herein may be administered as a formulation in association with one or more pharmaceutically acceptable carriers. The choice of carrier will to a large extent depend on factors such as the particular mode of administration, the effect of the carrier on solubility and stability, and the nature of the dosage form. Pharmaceutical compositions suitable for the delivery of Compounds I-Lu, Ia-Lu, I-Ac, or Ia-Ac and/or PSMA ligand-imaging conjugates described herein and methods for their preparation will be readily apparent to those skilled in the art. Further, the shielding agents described herein may be administered as a formulation in association with one or more pharmaceutically acceptable carriers. The choice of carrier will to a large extent depend on factors such as the particular mode of administration, the effect of the carrier on solubility and stability, and the nature of the dosage form. Such compositions and methods for their preparation may be found, for example, in Remington: The Science & Practice of Pharmacy, 21th Edition (Lippincott Williams & Wilkins, 2005), incorporated herein by reference.

In one illustrative aspect, a pharmaceutically acceptable carrier includes any and all solvents, dispersion media, coatings, antibacterial and antifungal agents, isotonic and absorption delaying agents, and the like, and combinations thereof, that are physiologically compatible. In some embodiments, the carrier is suitable for parenteral administration. Pharmaceutically acceptable carriers include sterile aqueous solutions or dispersions and sterile powders for the extemporaneous preparation of sterile injectable solutions or dispersions. Supplementary active compounds can also be incorporated into compositions of the present disclosure.

In various embodiments, liquid formulations may include suspensions and solutions. Such formulations may comprise a carrier, for example, water, ethanol, polyethylene glycol, propylene glycol, methylcellulose or a suitable oil, and one or more emulsifying agents and/or suspending agents. Liquid formulations may also be prepared by the reconstitution of a solid.

In one embodiment, an aqueous suspension may contain the active materials in admixture with appropriate excipients. Such excipients are suspending agents, for example, sodium carboxymethylcellulose, methylcellulose, hydroxypropylmethylcellulose, sodium alginate, polyvinylpyrrolidone, gum tragacanth and gum acacia; dispersing or wetting agents which may be a naturally-occurring phosphatide, for example, lecithin; a condensation product of an alkylene oxide with a fatty acid, for example, polyoxyethylene stearate; a condensation product of ethylene oxide with a long chain aliphatic alcohol, for example, heptadecaethyleneoxycetanol; a condensation product of ethylene oxide with a partial ester derived from fatty acids and a hexitol such as polyoxyethylene sorbitol monooleate; or a condensation product of ethylene oxide with a partial ester derived from fatty acids and hexitol anhydrides, for example, polyoxyethylene sorbitan monooleate. The aqueous suspensions may also contain one or more preservatives, for example, ascorbic acid, ethyl, n-propyl, or p-hydroxybenzoate; or one or more coloring agents.

In one illustrative embodiment, dispersible powders and granules suitable for preparation of an aqueous suspension by the addition of water provide the active ingredient in admixture with a dispersing or wetting agent, suspending agent and one or more preservatives. Additional excipients, for example, coloring agents, may also be present.

Suitable emulsifying agents may be naturally-occurring gums, for example, gum acacia or gum tragacanth; naturally-occurring phosphatides, for example, soybean lecithin; and esters including partial esters derived from fatty acids and hexitol anhydrides, for example, sorbitan mono-oleate, and condensation products of the said partial esters with ethylene oxide, for example, polyoxyethylene sorbitan monooleate.

In other embodiments, isotonic agents, for example, sugars, polyalcohols such as mannitol, sorbitol, or sodium chloride can be included in the composition. Prolonged absorption of injectable compositions can be brought about by including in the composition an agent which delays absorption, for example, monostearate salts and gelatin.

Illustrative formats for oral administration include tablets, capsules, elixirs, syrups, and the like.

Depending upon the cancer type as described herein, the route of administration and/or whether Compound I-Lu ,or -Lu, I-Ac, or Ia-Ac and/or PSMA ligand-imaging conjugates are administered locally or systemically, a wide range of permissible dosages are contemplated herein, including doses falling in the range from about 1 MBq to about 4 MBq of I-Ac or Ia-Ac. In some embodiments, permissible dosages for I-Lu or Ia-Lu are contemplated herein in the units GBq, including doses falling in the range from about 2 GBq to about 13 GBq. The dosages may be single or divided, and may administered according to a wide variety of protocols, including q.d., b.i.d., t.i.d., or even every other day, biweekly (b.i.w.), once a week, once a month, once a quarter, and the like. In each of these cases it is understood that the therapeutically effective amounts described herein correspond to the instance of administration, or alternatively to the total daily, weekly, monthly, or quarterly dose, as determined by the dosing protocol. In some embodiments, a combination of compounds of the formula I-Lu or Ia-Lu, and I-Ac or Ia-Ac can be administered on independent schedules of once, or once per week, or once every two weeks, or once every three weeks, or once every four weeks, or once every five weeks, or once every six weeks, or once every seven weeks, or once every eight weeks, and the like

In one aspect, Compound I-Lu, or -Lu, I-Ac, or Ia-Ac and/or PSMA ligand-imaging conjugates as described herein may be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. Suitable means for parenteral administration include needle (including microneedle) injectors, needle-free injectors and infusion techniques.

In one illustrative aspect, parenteral formulations are typically aqueous solutions which may contain carriers or excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water. In other embodiments, any of the liquid formulations described herein may be adapted for parenteral administration of the Compound 1 or PSMA ligand-imaging conjugates described herein. The preparation of parenteral formulations under sterile conditions, for example, by lyophilization under sterile conditions, may readily be accomplished using standard pharmaceutical techniques well known to those skilled in the art. In one embodiment, the solubility of Compound I-Lu, or -Lu, I-Ac, or Ia-Ac and/or PSMA ligand-imaging conjugates used in the preparation of a parenteral formulation may be increased by the use of appropriate formulation techniques, such as the incorporation of solubility-enhancing agents.

In various embodiments, formulations for parenteral administration may be formulated for immediate and/or modified release. In one illustrative aspect, the active agents of the present disclosure (i.e., Compound I-Lu, or -Lu, I-Ac, or Ia-Ac and/or PSMA ligand-imaging conjugates) may be administered in a time release formulation, for example in a composition which includes a slow release polymer. The active Compound I-Lu, or -Lu, I-Ac, or Ia-Ac and/or PSMA ligand-imaging conjugates can be prepared with carriers that will protect Compound I-Lu, or -Lu, I-Ac, or Ia-Ac and/or PSMA ligand-imaging conjugates against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PGLA). Methods for the preparation of such formulations are generally known to those skilled in the art. In another embodiment, Compound I-Lu, or -Lu, I-Ac, or Ia-Ac and/or PSMA ligand-imaging conjugates described herein or compositions comprising the Compound I-Lu, or -Lu, I-Ac, or Ia-Ac and/or PSMA ligand-imaging conjugates may be continuously administered, where appropriate.

In one embodiment, a kit is provided. If a Compound I-Lu, or -Lu, I-Ac, or Ia-Ac and/or PSMA ligand-imaging conjugate is to be administered in combination with a shielding agent described herein, two or more pharmaceutical compositions may be combined in the form of a kit suitable for sequential administration or co-administration of the compositions. Such a kit comprises two or more separate pharmaceutical compositions, at least one of which contains Compound I-Lu, or -Lu, I-Ac, or Ia-Ac and/or PSMA ligand-imaging conjugate described herein, and another contains at least one shielding agent as described herein, and means for separately retaining the compositions, such as a container, divided bottle, or divided foil packet. In another embodiment, compositions comprising one or more of Compound I-Lu , or -Lu, I-Ac, or Ia-Ac and/or PSMA ligand-imaging conjugates described herein, and at least one shielding agent as described herein, in containers having labels that provide instructions for use in patient selection and/or treatment are provided.

In one embodiment, sterile injectable solutions can be prepared by incorporating the active agent in the required amount in an appropriate solvent with one or a combination of ingredients described above, as required, followed by filtered sterilization. Typically, dispersions are prepared by incorporating the active Compound I-Lu, or -Lu, I-Ac, or Ia-Ac and/or PSMA ligand-imaging conjugate, or a shielding agent as described herein, into a sterile vehicle which contains a dispersion medium and any additional ingredients of those described above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof, or the ingredients may be sterile-filtered together.

The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. In one embodiment, the proper fluidity can be maintained, for example, by the use of a coating such as lecithin, by the maintenance of the required particle size in the case of dispersion and by the use of surfactants.

Dose levels of Compounds I-Lu or Ia-Lu, and I-Ac or Ia-Ac can be measured in GBq and MBq, respectively. In some embodiments, a therapeutically effective amount of I-Lu or Ia-Lu is from about 2 GBq to about 20 GBq. In some embodiments, a therapeutically effective amount of I-Lu or Ia-Lu is from about 2 GBq to about 13 GBq. In some embodiments, a therapeutically effective amount of I-Lu or Ia-Lu is from about 4 GBq to about 11 GBq. In some embodiments, a therapeutically effective amount of I-Lu or Ia-Lu is from about 5 GBq to about 10 GBq. In some embodiments, a therapeutically effective amount of I-Lu or Ia-Lu is from about 6 GBq to about 9 GBq. In some embodiments, a therapeutically effective amount of I-Lu or Ia-Lu is from about 6 GBq to about 8 GBq. In some embodiments, a therapeutically effective amount of I-Lu or Ia-Lu is from about 6.5 GBq to about 8.5 GBq. In some embodiments, a therapeutically effective amount of I-Lu or Ia-Lu is from about 7 GBq to about 8 GBq. In some embodiments, a therapeutically effective amount of I-Lu or Ia-Lu is about 7.4 GBq. In some embodiments, the total dose of I-Lu or Ia-Lu ranges from about 15 GBq to about 200 GBq. In some embodiments, the total dose of I-Lu or Ia-Lu ranges from about 25 GBq to about 185 GBq. In some embodiments, the total dose of I-Lu or Ia-Lu ranges from about 35 GBq to about 150 GBq. In some embodiments, the total dose of I-Lu or Ia-Lu ranges from about 40 GBq to about 100 GBq. In some embodiments, the total dose of I-Lu, or Ia-Lu is about 44 GBq. In some embodiments, the maximum duration of treatment of a subject is about 19 to 23 months.

In some embodiments, a therapeutically effective amount of I-Lu or Ia-Lu is from 2 GBq to 20 GBq. In some embodiments, a therapeutically effective amount of I-Lu or Ia-Lu is from 2 GBq to 13 GBq. In some embodiments, a therapeutically effective amount of I-Lu or Ia-Lu is from 4 GBq to 11 GBq. In some embodiments, a therapeutically effective amount of I-Lu or Ia-Lu is from 5 GBq to 10 GBq. In some embodiments, a therapeutically effective amount of I-Lu or Ia-Lu is from 6 GBq to 9 GBq. In some embodiments, a therapeutically effective amount of I-Lu or Ia-Lu is from 6 GBq to 8 GBq. In some embodiments, a therapeutically effective amount of I-Lu or Ia-Lu is from 6.5 GBq to 8.5 GBq. In some embodiments, a therapeutically effective amount of I-Lu or Ia-Lu is from 7 GBq to 8 GBq. In some embodiments, a therapeutically effective amount of I-Lu or Ia-Lu is 7.4 GBq. In some embodiments, the total dose of I-Lu or Ia-Lu ranges from 15 GBq to 200 GBq. In some embodiments, the total dose of I-Lu or Ia-Lu ranges from 25 GBq to 185 GBq. In some embodiments, the total dose of I-Lu or Ia-Lu ranges from 35 GBq to 150 GBq. In some embodiments, the total dose of I-Lu or Ia-Lu ranges from 40 GBq to 100 GBq. In some embodiments, the total dose of I-Lu, or Ia-Lu is 44 GBq. In some embodiments, the maximum duration of treatment of a subject is 19 to 23 months.

In some embodiments, a therapeutically effective amount of I-Ac or Ia-Ac is from about 1 MBq to about 20 MBq. In some embodiments, a therapeutically effective amount of I-Ac or Ia-Ac is from about 1 MBq to about 10 MBq. In some embodiments, a therapeutically effective amount of I-Ac or Ia-Ac is from about 4 MBq to about 14 MBq. In some embodiments, a therapeutically effective amount of I-Ac or Ia-Ac is from about 5 MBq to about 10 MBq. In some embodiments, a therapeutically effective amount of I-Ac or Ia-Ac is from about 6 MBq to about 8 MBq. In some embodiments, a therapeutically effective amount of I-Ac or Ia-Ac is from about 5 MBq to about 7 MBq. In some embodiments, a therapeutically effective amount of I-Ac or Ia-Ac is from about 1 MBq to about 4 MBq. In some embodiments, a therapeutically effective amount of I-Ac or Ia-Ac is from about 2 MBq to about 3 MBq. In some embodiments, a therapeutically effective amount of I-Ac or Ia-Ac is about 5 MBq. In some embodiments, a therapeutically effective amount of I-Ac or Ia-Ac is about 2.5 MBq.

In some embodiments, a therapeutically effective amount of I-Ac or Ia-Ac is from 1 MBq to 20 MBq. In some embodiments, a therapeutically effective amount of I-Ac or Ia-Ac is from 1 MBq to 10 MBq. In some embodiments, a therapeutically effective amount of I-Ac or Ia-Ac is from 4 MBq to 14 MBq. In some embodiments, a therapeutically effective amount of I-Ac or Ia-Ac is from 5 MBq to 10 MBq. In some embodiments, a therapeutically effective amount of I-Ac or Ia-Ac is from 6 MBq to 8 MBq. In some embodiments, a therapeutically effective amount of I-Ac or Ia-Ac is from 5 MBq to 7 MBq. In some embodiments, a therapeutically effective amount of I-Ac or Ia-Ac is from 1 MBq to 4 MBq. In some embodiments, a therapeutically effective amount of I-Ac or Ia-Ac is from 2 MBq to 3 MBq. In some embodiments, a therapeutically effective amount of I-Ac or Ia-Ac is 5 MBq. In some embodiments, a therapeutically effective amount of I-Ac or Ia-Ac is 2.5 MBq.

The PSMA ligand-imaging conjugates, Compounds I-Lu, I-Ac, Ia-Lu, and Ia-Ac, and shielding agents described herein may contain one or more chiral centers, or may otherwise be capable of existing as multiple stereoisomers. Accordingly, it is to be understood that the present disclosure includes pure stereoisomers as well as mixtures of stereoisomers, such as enantiomers, diastereomers, and enantiomerically or diastereomerically enriched mixtures. The PSMA ligand-imaging conjugates, Compounds I-Lu, I-Ac, Ia-Lu, and Ia-Ac, and shielding agents described herein may be capable of existing as geometric isomers. Accordingly, it is to be understood that the present disclosure includes pure geometric isomers or mixtures of geometric isomers. For example, shielding agent 1c is of the formula

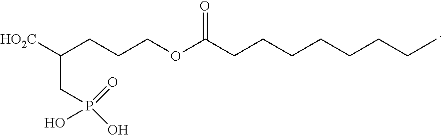 [SMILES: CCCCCCCCC(=O)OCCCC(CP(=O)(O)O)C(=O)O]

A person skilled in the art will recognize that shielding agent 1c has a chiral center and can thus exist in two enantiomeric forms. A person skilled in the art will recognize that the two enantiomers of shielding agent 1c are

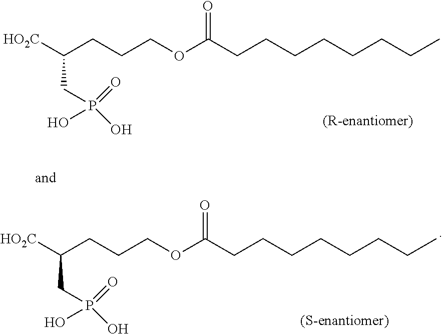 [SMILES: CCCCCCCCC(=O)OCCC[C@H](CP(=O)(O)O)C(=O)O ; CCCCCCCCC(=O)OCCC[C@@H](CP(=O)(O)O)C(=O)O]

It will be appreciated that the disclosure of shielding agent 1c as shown above, also includes disclosure of the R-enantiomer and S-enantiomer of shielding agent 1c. Similarly, the disclosure of other shielding agents, PSMA ligand-imaging agents, and Compounds I-Lu, I-Ac, Ia-Lu, and Ia-Ac also includes disclosure of their respective enantiomers, diasteriomers, and the like.

It is appreciated that the PSMA ligand-imaging conjugates and Compounds I-Lu, I-Ac, and Ia-Lu, Ia-Ac described herein may exist in unsolvated forms as well as solvated forms, including hydrated forms. In general, the solvated forms are equivalent to unsolvated forms and are encompassed within the scope of the present disclosure. The PSMA ligand-imaging conjugates and Compounds I-Lu, I-Ac, Ia-Lu, and Ia-Ac described herein may exist in multiple crystalline or amorphous forms. In general, all physical forms are equivalent for the uses contemplated by the present disclosure and are intended to be within the scope of the present disclosure.

In another embodiment, compositions and/or dosage forms for administration of Compounds I-Lu, Ia-Lu, I-Ac, or Ia-Ac are prepared from Compounds I-Lu, Ia-Lu, I-Ac, or Ia-Ac with purity of at least about 90%, or about 95%, or about 96%, or about 97%, or about 98%, or about 99%, or about 99.5%. In another embodiment, compositions and or dosage forms for administration of Compounds I-Lu, Ia-Lu, I-Ac, or Ia-Ac are prepared from Compounds I-Lu, Ia-Lu, I-Ac, or Ia-Ac with a purity of at least 90%, or at least 95%, or at least 96%, or at least 97%, or at least 98%, or at least 99%, or at least 99.5%.

In another embodiment, compositions and/or dosage forms for administration of the PSMA ligand-imaging conjugate are prepared from the PSMA ligand-imaging conjugate with a purity of at least about 90%, or about 95%, or about 96%, or about 97%, or about 98%, or about 99%, or about 99.5%. In another embodiment, compositions and or dosage forms for administration of the PSMA ligand-imaging conjugate are prepared from the PSMA ligand-imaging conjugate with a purity of at least 90%, or at least 95%, or at least 97%, or at least 98%, or at least 99%, or at least 99.5%.

In another embodiment, compositions and/or dosage forms for administration of radiolabeled PSMA ligand-imaging conjugate are prepared from the PSMA ligand-imaging conjugate with a radiochemical purity of at least about 90%, or about 95%, or about 96%, or about 97%, or about 98%, or about 99%, or about 99.5%. In another embodiment, compositions and or dosage forms for administration of the PSMA ligand-imaging conjugate are prepared from the PSMA ligand-imaging conjugate with a purity of at least 90%, or at least 95%, or at least 96%, or at least 97%, or at least 98%, or at least 99%, or at least 99.5%.

The purity of Compounds I-Lu, I-Ac, Ia-Lu, and Ia-Ac or the PSMA ligand-imaging conjugates described herein may be measured using any conventional technique, including various chromatography or spectroscopic techniques, such as high pressure or high performance liquid chromatography (HPLC), nuclear magnetic resonance spectroscopy, TLC, UV absorbance spectroscopy, fluorescence spectroscopy, and the like.

In another embodiment, Compounds I-Lu, I-Ac, Ia-Lu, and Ia-Ac or PSMA ligand-imaging conjugate described herein is provided in a sterile container or package.

In one aspect, a clinical benefit of the patient to treatment with a combination of Compounds I-Lu or Ia-Lu, and I-Ac or Ia-Ac can be characterized as overall survival (OS). As used herein, the term “overall survival (OS)” means the time from the date of randomization to the date of death from any cause.

In one aspect, a clinical benefit of the patient to treatment with Compound I-Lu, Ia-Lu, I-Ac, or Ia-Ac can be characterized utilizing Response Evaluation Criteria in Solid Tumors (RECIST) criteria. Illustratively, the criteria have been adapted from the original WHO Handbook (3), taking into account the measurement of the longest diameter for all target lesions: complete response, (CR)—the disappearance of all target lesions; partial response (PR)—at least a 30% decrease in the sum of the longest diameter of target lesions, taking as reference the baseline sum longest diameter; stable disease (SD)—neither sufficient shrinkage to qualify for partial response nor sufficient increase to qualify for progressive disease, taking as reference the smallest sum longest diameter since the treatment started; progressive disease (PD)—at least a 20% increase in the sum of the longest diameter of target lesions, taking as reference the smallest sum longest diameter recorded since the treatment started or the appearance of one or more new lesions. In another aspect overall disease response rate (ORR) is a clinical benefit and is calculated as the percent of patients who achieve a best response of CR or PR. Overall disease control rate (DCR) can be another clinical benefit and is calculated as the percent of patients who achieve a best response of CR, PR, or SD. In some embodiments, the response can be disease control rate (DCR) as measured by RECIST v1.1 criteria.

In another aspect, a clinical benefit of the patient to treatment with Compound I-Lu, Ia-Lu, I-Ac, or Ia-Ac can be characterized as radiographic progression-free survival (rPFS). As used herein, “radiographic progression-free survival (rPFS)” means the time from the date of randomization to the date of radiographic disease progression as outlined in Prostate Cancer Working Group 3 (PCWG3) Guidelines or death from any cause. See, for example, Scher H I, Morris M J, Stadler W M, Higano C, Basch E, Fizazi K, et al. Trial Design and Objectives for Castration-Resistant Prostate Cancer: Updated Recommendations from the Prostate Cancer Clinical Trials Work Group 3. J Clin Oncol 2016; 34(12):1402-18. In another aspect, a clinical benefit of the patient to treatment with Compound 1 can be characterized as time to a first symptomatic skeletal event (SSE). It will be appreciated that symptomatic skeletal event means a clinically significant pathological fracture, surgery or radiation to bone, or spinal cord compression. As used herein, “time to a first symptomatic skeletal event” means date of randomization to the date of first new symptomatic pathological bone fracture, spinal cord compression, tumor-related orthopedic surgical intervention, or requirement for radiation therapy to relieve bone pain, whichever occurs first.

In one illustrative example overall survival is the time to death for a given patient defined as the number of days from the first day the patient received protocol treatment (C1D1) to the date of the patient's death. All events of death can be included, regardless of whether the event occurred while the patient was still taking the study drug or after the patient discontinued the study drug. If a patient has not died, then the data can be censored at the last study visit, or the last contact date, or the date the patient was last known to be alive, whichever is last.

Alternatively, a clinical benefit of the patient as a result of treatment with Compound I-Lu, Ia-Lu, I-Ac, or Ia-Ac can be characterized as inhibition of tumor growth which can be identified in a patient through, for example, follow-up imaging of the patient's cancer after treatment with the Compound. For example, inhibition of tumor growth can be characterized by measuring the size of tumors in a patient after administration of Compound I-Lu, Ia-Lu, I-Ac, or Ia-Ac according to any of the imaging techniques described herein, where the inhibition of tumor growth is indicated by a stable tumor size, or by a reduction in tumor size. It will be appreciated that the identification of inhibition of tumor growth can be accomplished using a variety of techniques, and is not limited to the imaging methods described herein (e.g CT, MRI, PET imaging, SPECT imaging or chest x-ray).

The embodiments described in the Detailed Description and Summary can be combined with each of the following numbered paragraphs to the extent that such embodiments are not in conflict with one another:

1. A compound selected from the group consisting of

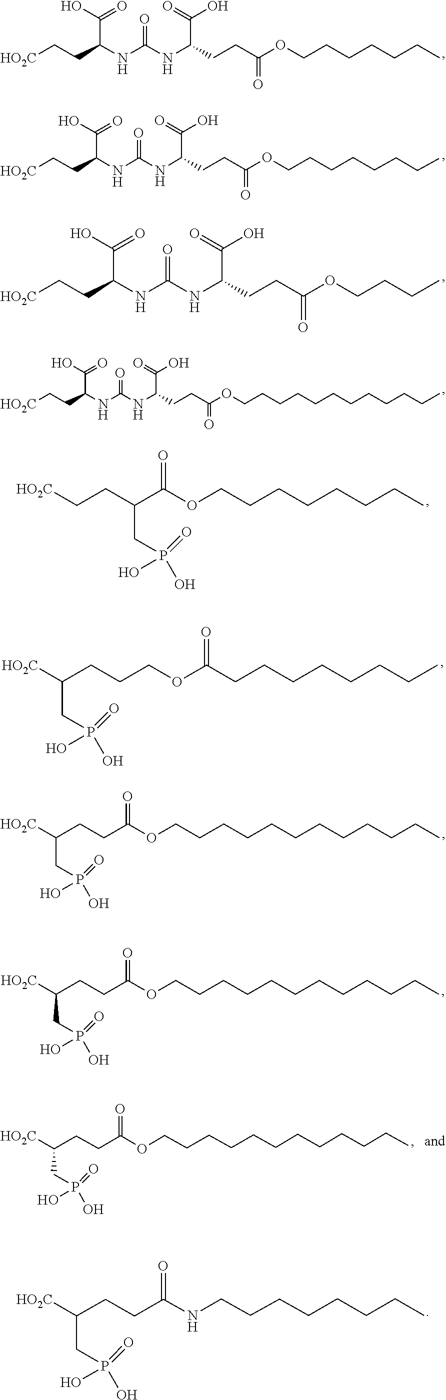

2. A method for treating a cancer in a patient comprising administering a therapeutically effective amount of a radiolabeled therapeutic in combination with a an effective amount of a shielding agent.

3. The method of clause 2, wherein the radiolabeled therapeutic is Compound Ia-Lu or Ia-Ac.

4. The method of clause 2 or 3, wherein the cancer is a prostate cancer. 5. The method of any one of clauses 2 to 4, wherein the cancer is a metastatic prostate cancer.

6. The method of any one of clauses 2 to 4, wherein the cancer is a metastatic castration-resistant prostate cancer.

7. The method of any one of clauses 2 to 6, wherein the shielding agent is selected from the group consisting of

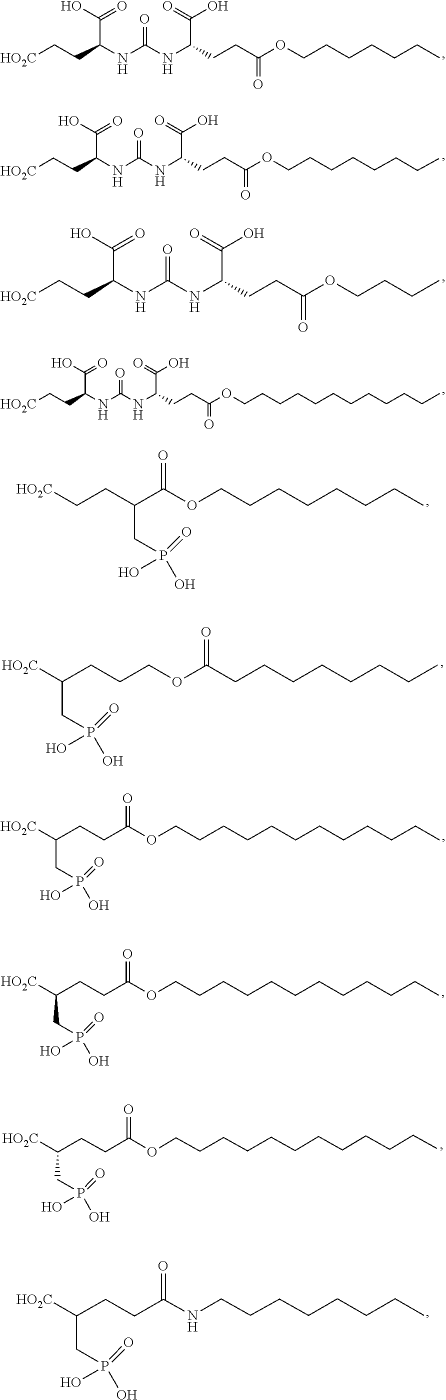

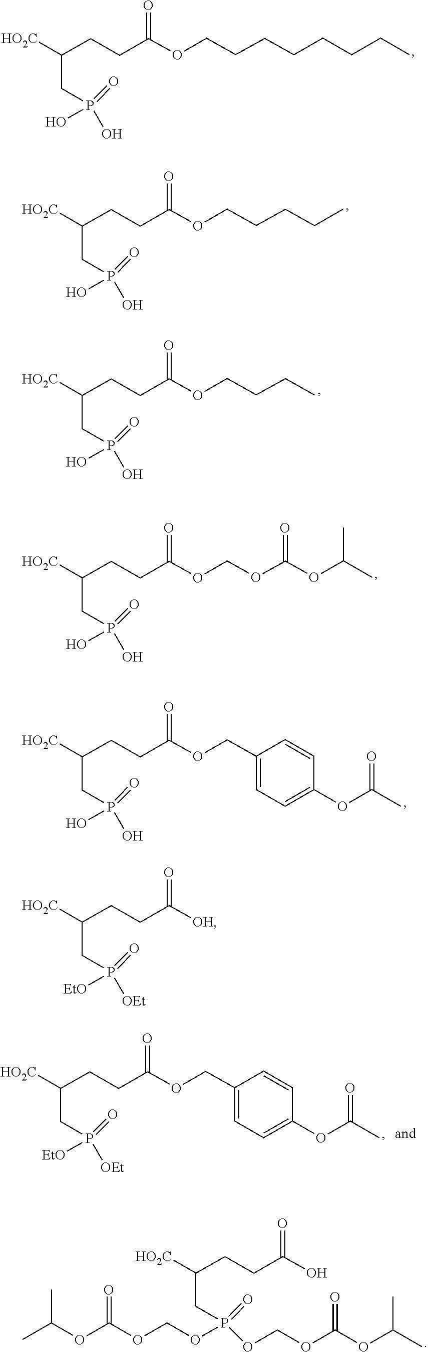

8. The method of any one of clauses 2 to 6, wherein the shielding agent is selected from the group consisting of

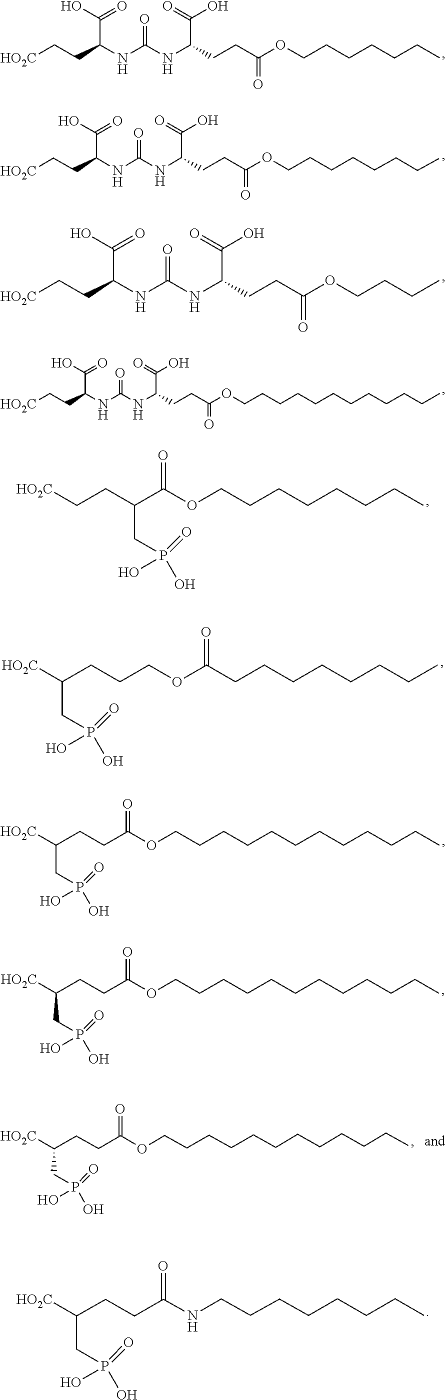

9. A compound selected from the group consisting of

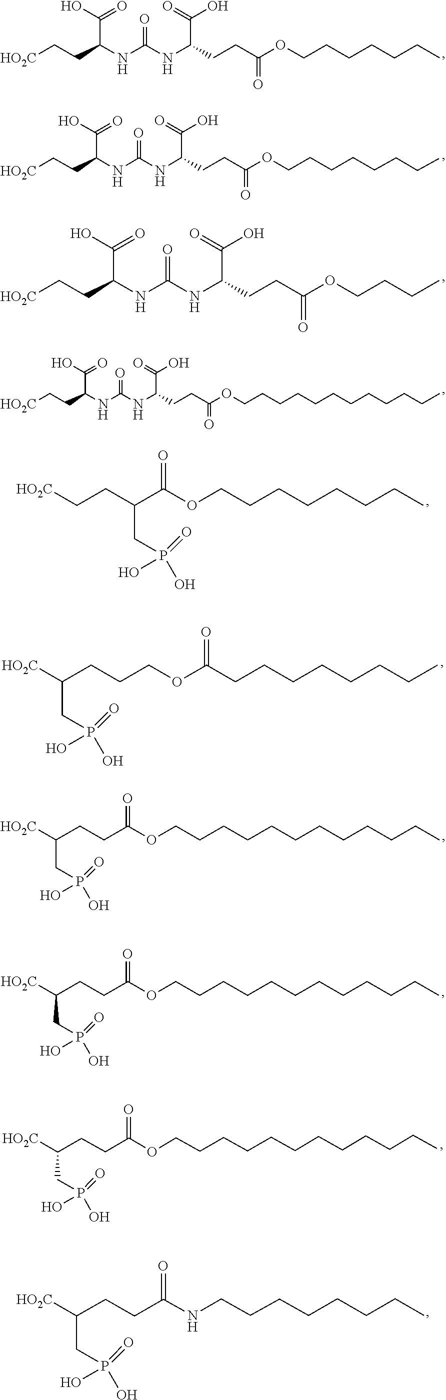

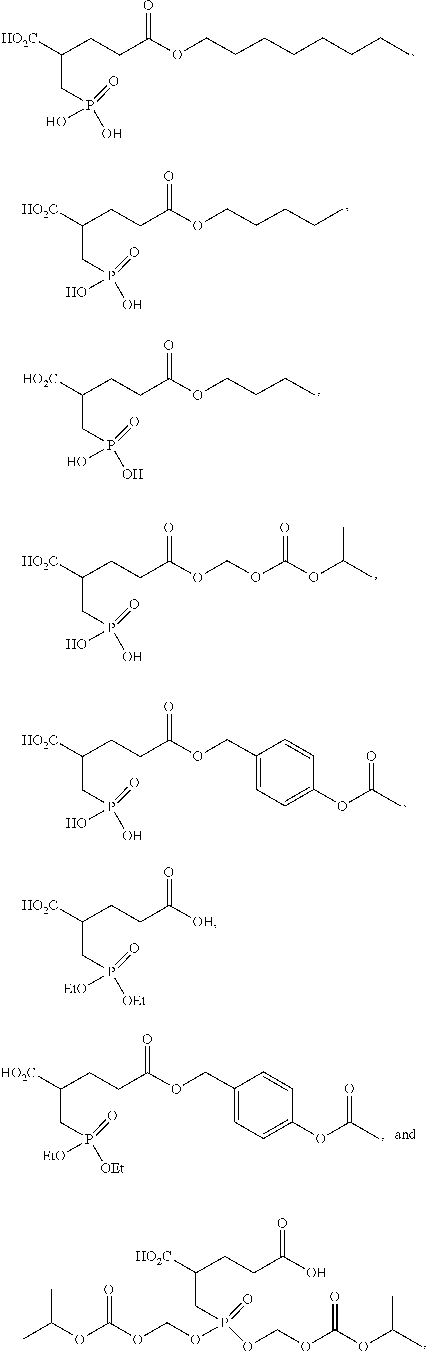

for treating cancer in a patient in need of such treatment in combination with a therapeutically effective amount of a radiolabeled therapeutic.

10. The compound of clause 9, wherein the radiolabeled therapeutic is Compound Ia-Lu or Ia-Ac.

11. The compound of clause 9 or 10, wherein the cancer is a prostate cancer.

12. The compound of any one of clauses 9 to 11, wherein the cancer is a metastatic prostate cancer.

13. The compound of any one of clauses 9 to 11, wherein the cancer is a metastatic castration-resistant prostate cancer.

14. Use of compound selected from the group consisting of

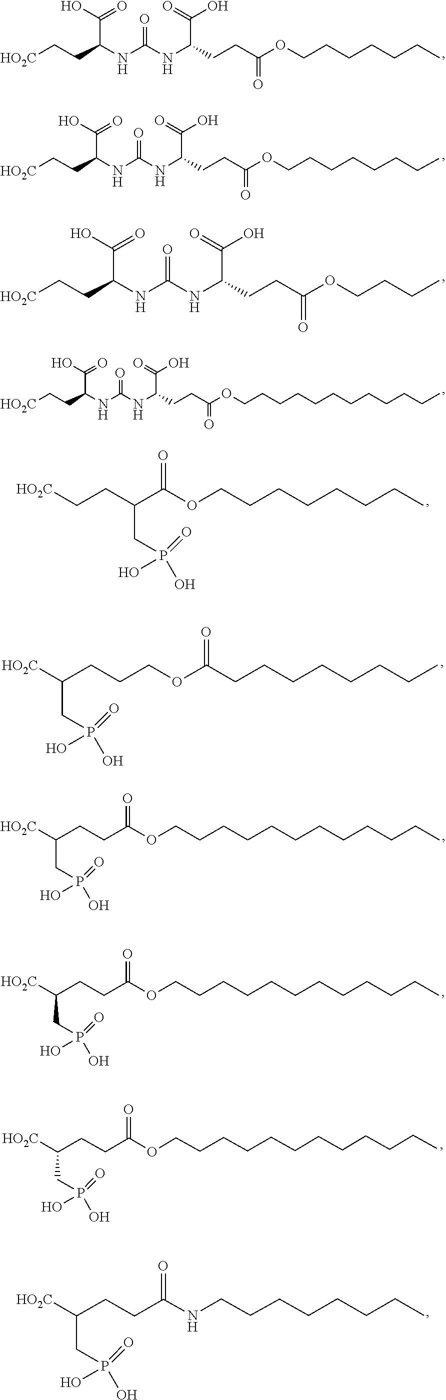

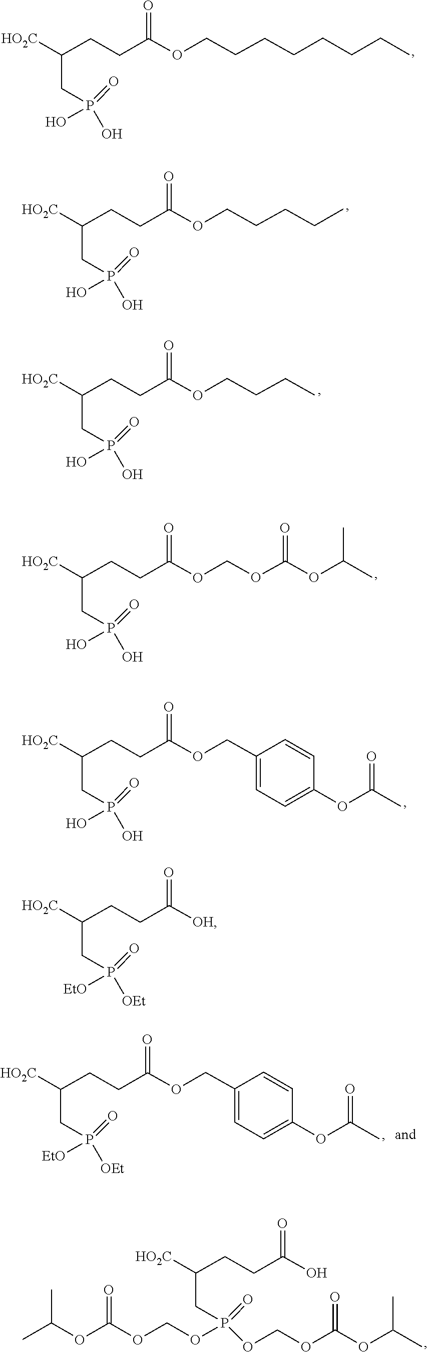

in the manufacture of a medicament for treating cancer in a patient in combination with a therapeutically effective amount of a radiolabeled therapeutic.

15. The use of clause 14, wherein the radiolabeled therapeutic is Compound Ia-Lu or Ia-Ac.

16. The use of clause 14 or 15, wherein the cancer is a prostate cancer.

17. The use of any one of clauses 14 to 16, wherein the cancer is a metastatic prostate cancer.

18. The use of any one of clauses 14 to 16, wherein the cancer is a metastatic castration-resistant prostate cancer.

19. A method for imaging a cancer in a patient comprising administering an effective amount of an imaging conjugate in combination with an effective amount of a shielding agent.

20. The method of clause 19, wherein the imaging is ^{99m}Tc labelled imaging conjugate 3a or ⁶⁷Ga or⁶⁸Ga labelled imaging conjugate 4.

21. The method of clause 19 or 20, wherein the cancer is a prostate cancer.

22. The method of any one of clauses 19 to 21, wherein the cancer is a metastatic prostate cancer.

23. The method of any one of clauses 19 to 22, wherein the cancer is a metastatic castration-resistant prostate cancer.

24. The method of any one of clauses 19 to 23, wherein the shielding agent is selected from the group consisting of

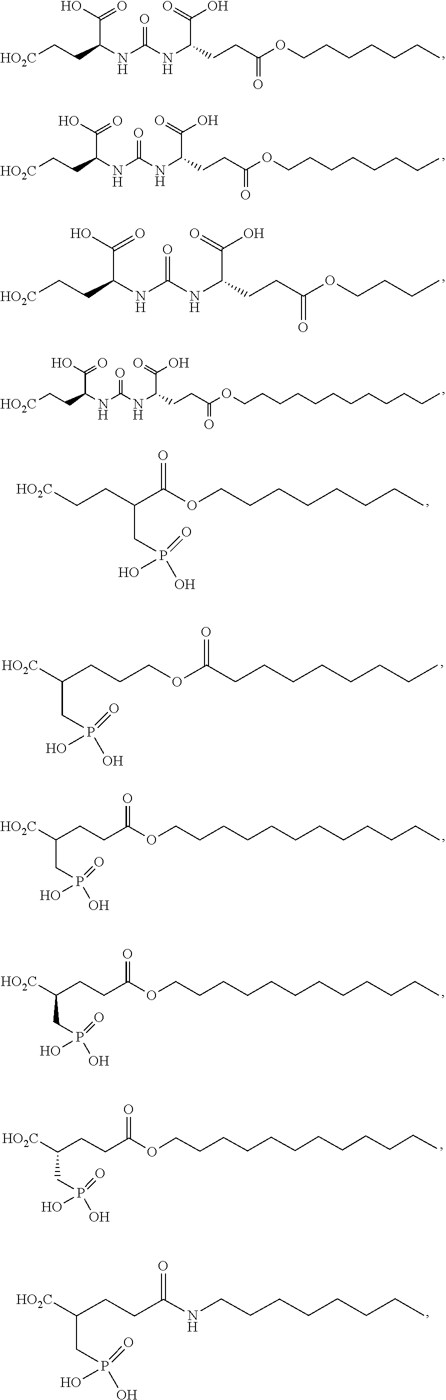

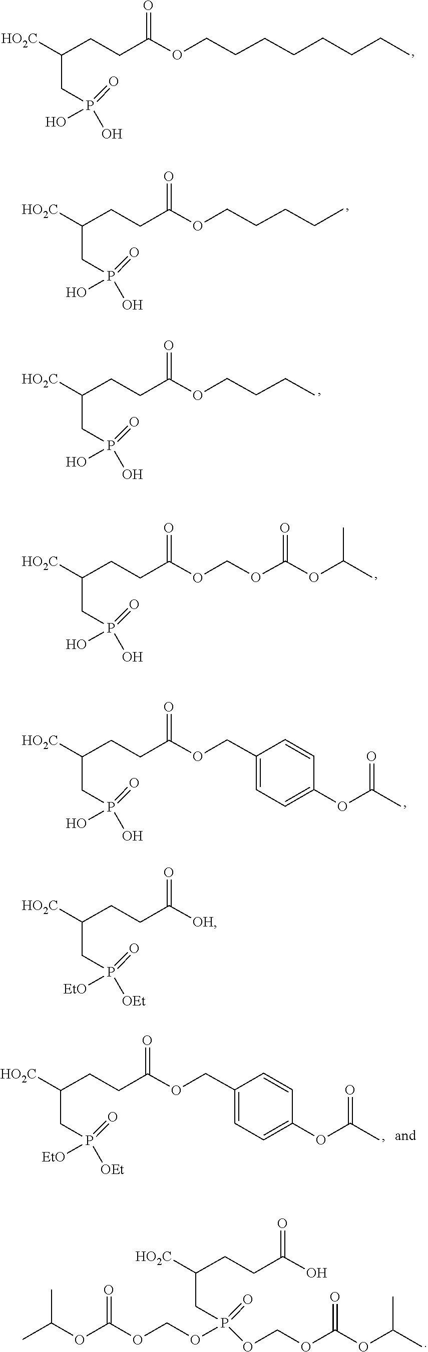

25. The method of any one of clauses 19 to 23, wherein the shielding agent is selected from the group consisting of

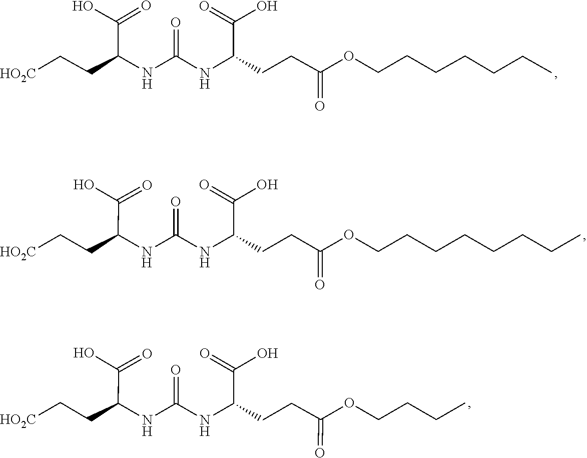 [SMILES: CCCCCCCOC(=O)CC[C@H](NC(=O)N[C@@H](CCC(=O)O)C(=O)O)C(=O)O ; CCCCCCCCOC(=O)CC[C@H](NC(=O)N[C@@H](CCC(=O)O)C(=O)O)C(=O)O ; CCCCOC(=O)CC[C@H](NC(=O)N[C@@H](CCC(=O)O)C(=O)O)C(=O)O]

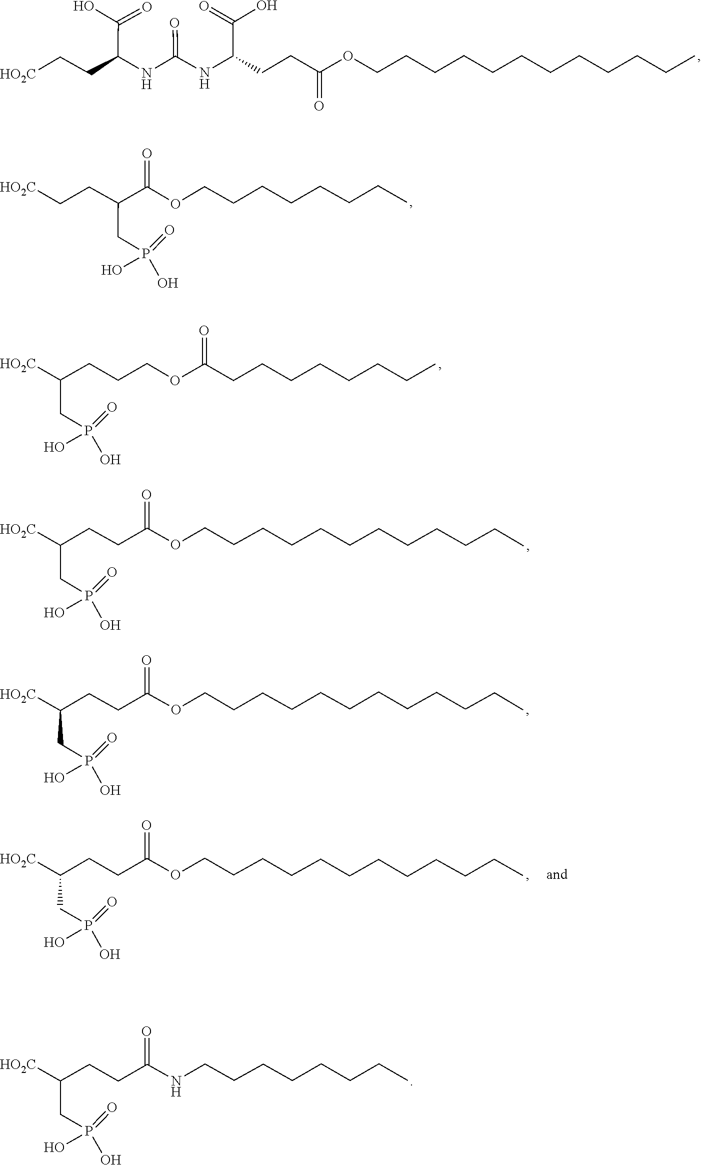

26. A compound selected from the group consisting of

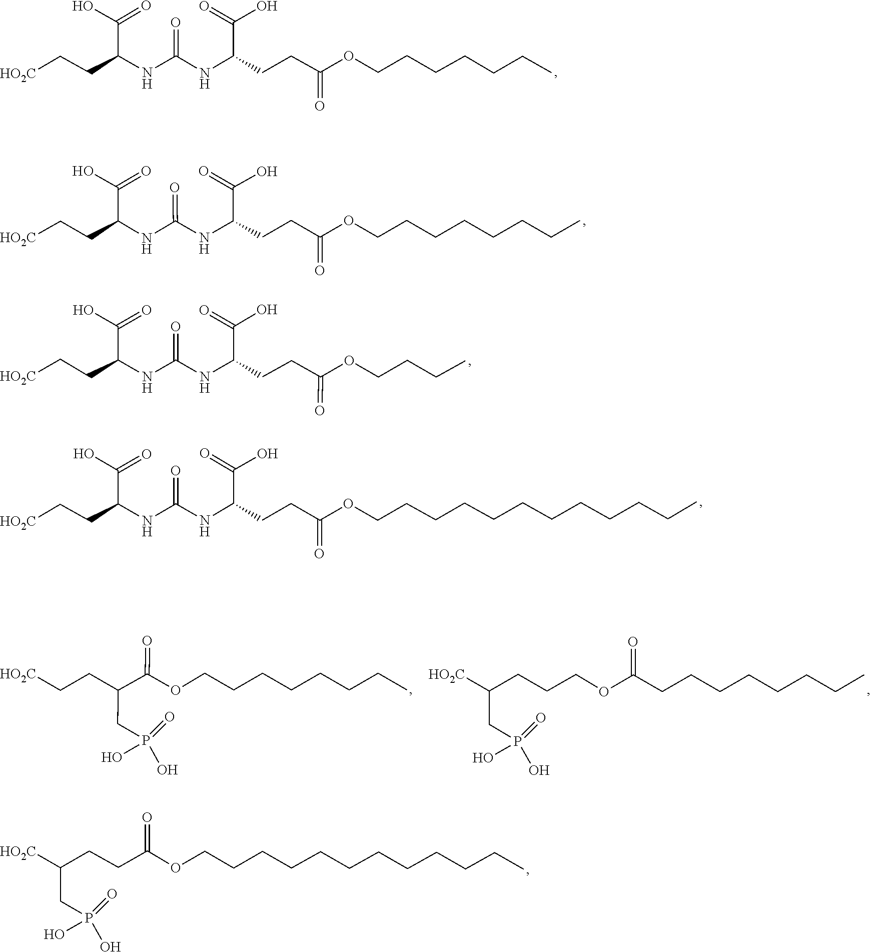

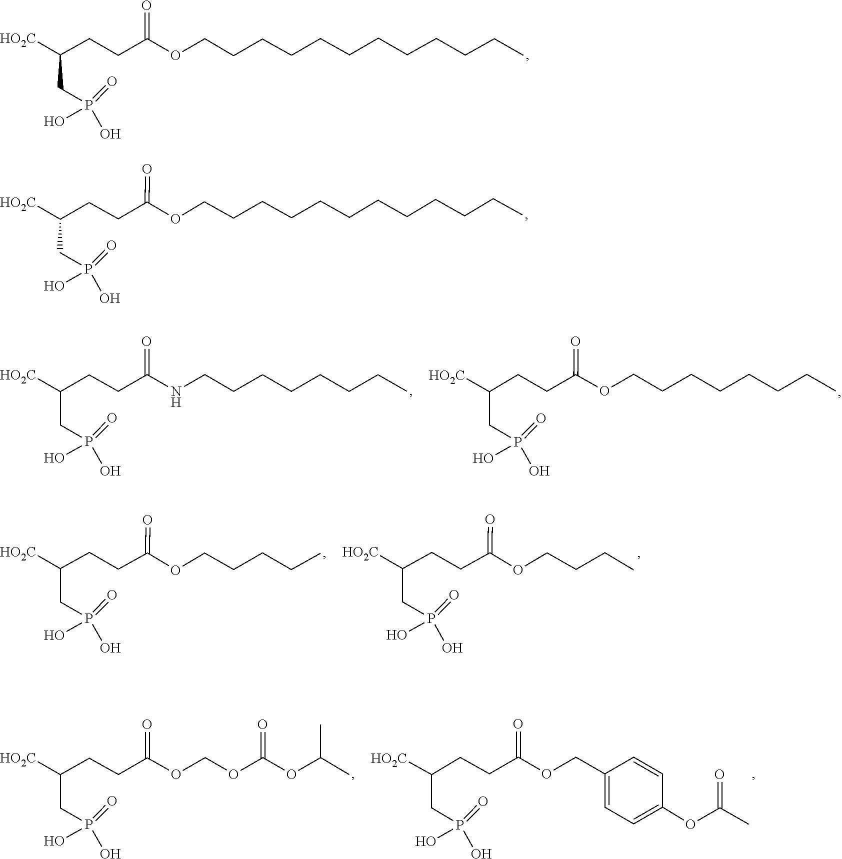

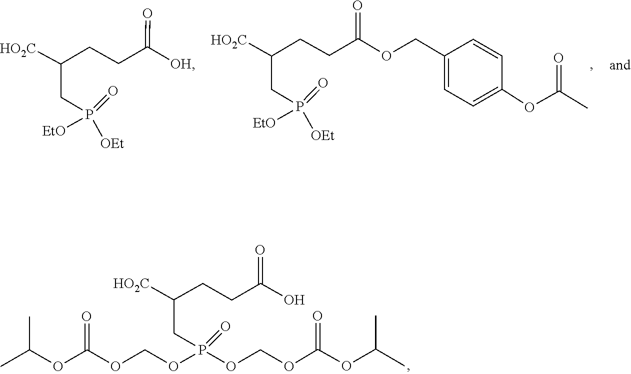 [SMILES: CCOP(=O)(CC(CCC(=O)O)C(=O)O)OCC ; CCOP(=O)(CC(CCC(=O)OCc1ccc(OC(C)=O)cc1)C(=O)O)OCC ; CC(C)OC(=O)OCOP(=O)(CC(CCC(=O)O)C(=O)O)OCOC(=O)OC(C)C]

for imaging cancer in a patient in need of such treatment in combination with an effective amount of an imaging conjugate.

27. The compound of clause 26, wherein the imaging conjugate is ^{99m}Tc labelled imaging conjugate 3a or ⁶⁷Ga or ⁶⁸Ga labelled imaging conjugate 4.

28. The compound of clause 26 or 27, wherein the cancer is a prostate cancer.

29. The compound of any one of clauses 26 to 28, wherein the cancer is a metastatic prostate cancer.

30. The compound of any one of clauses 26 to 28, wherein the cancer is a metastatic castration-resistant prostate cancer.

31. Use of compound selected from the group consisting of

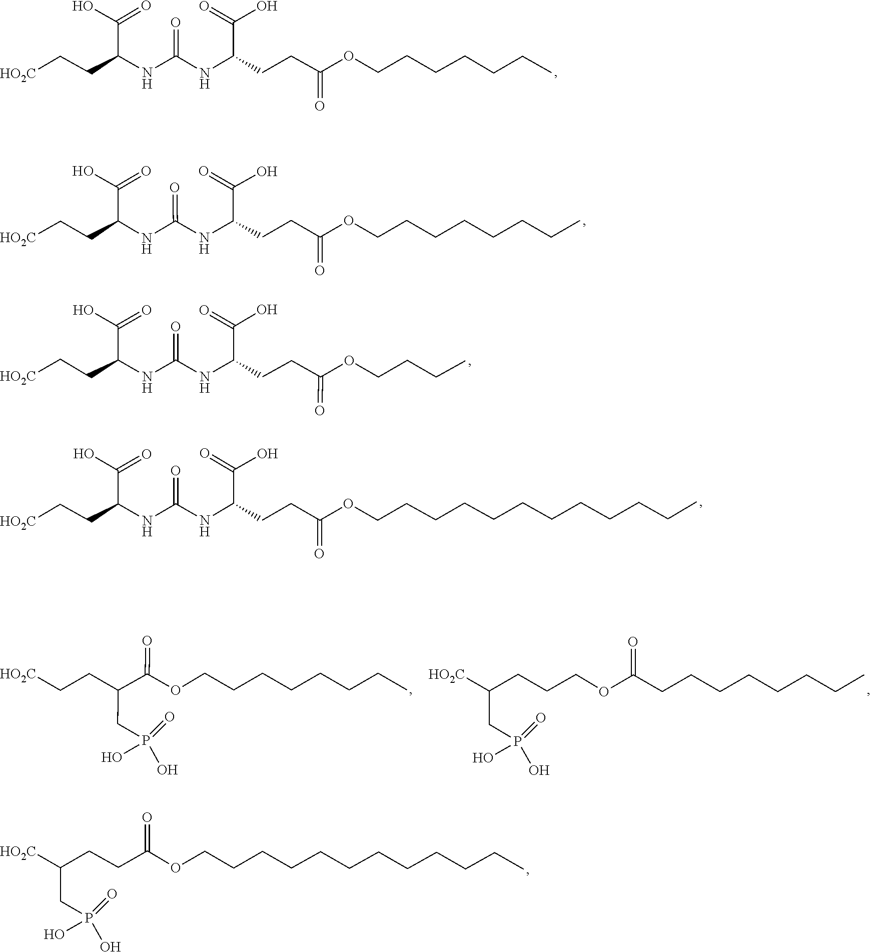

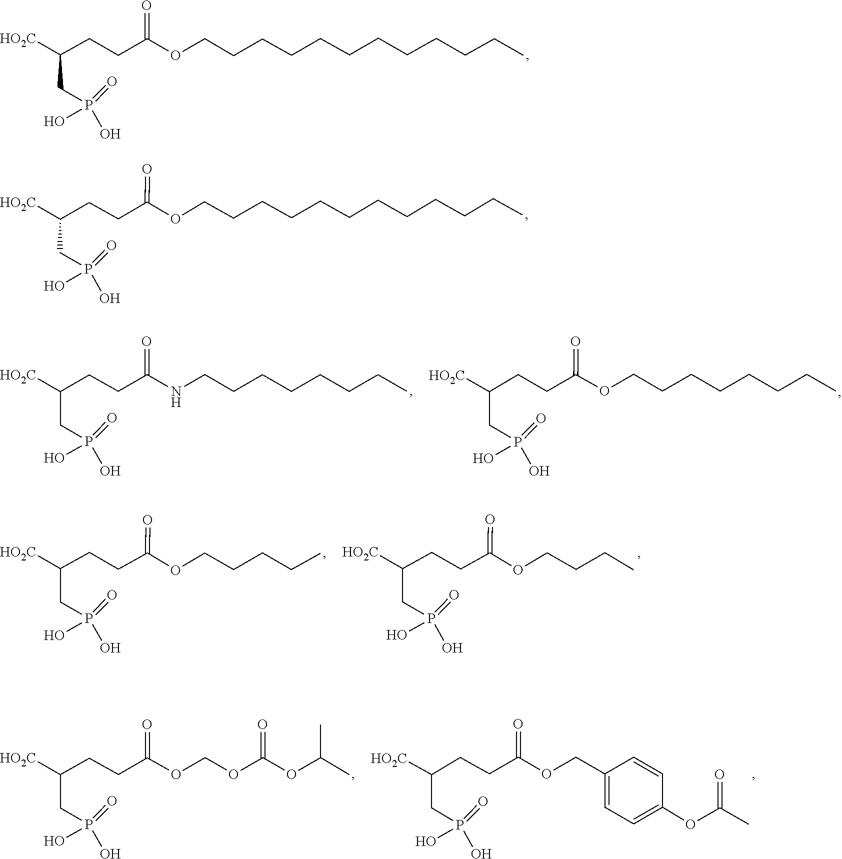

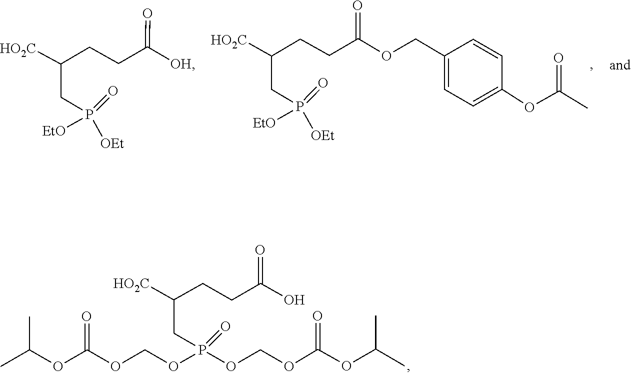 [SMILES: CCOP(=O)(CC(CCC(=O)O)C(=O)O)OCC ; CCOP(=O)(CC(CCC(=O)OCc1ccc(OC(C)=O)cc1)C(=O)O)OCC ; CC(C)OC(=O)OCOP(=O)(CC(CCC(=O)O)C(=O)O)OCOC(=O)OC(C)C]

in the manufacture of a medicament for imaging cancer in a patient in combination with an effective amount of n imaging conjugate.

32. The use of clause 31, wherein the imaging conjugate is ^{99m}Tc labelled imaging conjugate 3a or ⁶⁷Ga or ⁶⁸Ga labelled imaging conjugate 4.

33. The use of clause 31 or 32, wherein the cancer is a prostate cancer.

34. The use of any one of clauses 31 to 33, wherein the cancer is a metastatic prostate cancer.

35. The use of any one of clauses 31 to 34, wherein the cancer is a metastatic castration-resistant prostate cancer.

In another embodiment, the methods described herein include the following examples. The examples further illustrate additional features of the various embodiments of the present disclosure. However, it is to be understood that the examples are illustrative and are not to be construed as limiting other embodiments of the present disclosure. In addition, it is appreciated that other variations of the examples are included in the various embodiments of the present disclosure. In addition, it will be appreciated that all ranges described herein, such as those described in connection with the various embodiments, are exemplary and not intended to be limiting. One of skill in the art will appreciate that all ranges described by an lower and upper bound, such as about 1 to about 20, includes all possible values contained in the lower and upper bound, and includes all possible ranges of values available by the set of possible values contained in the lower and upper bound.

### EXAMPLES

### Example 1

### Preparation of Compound 1a

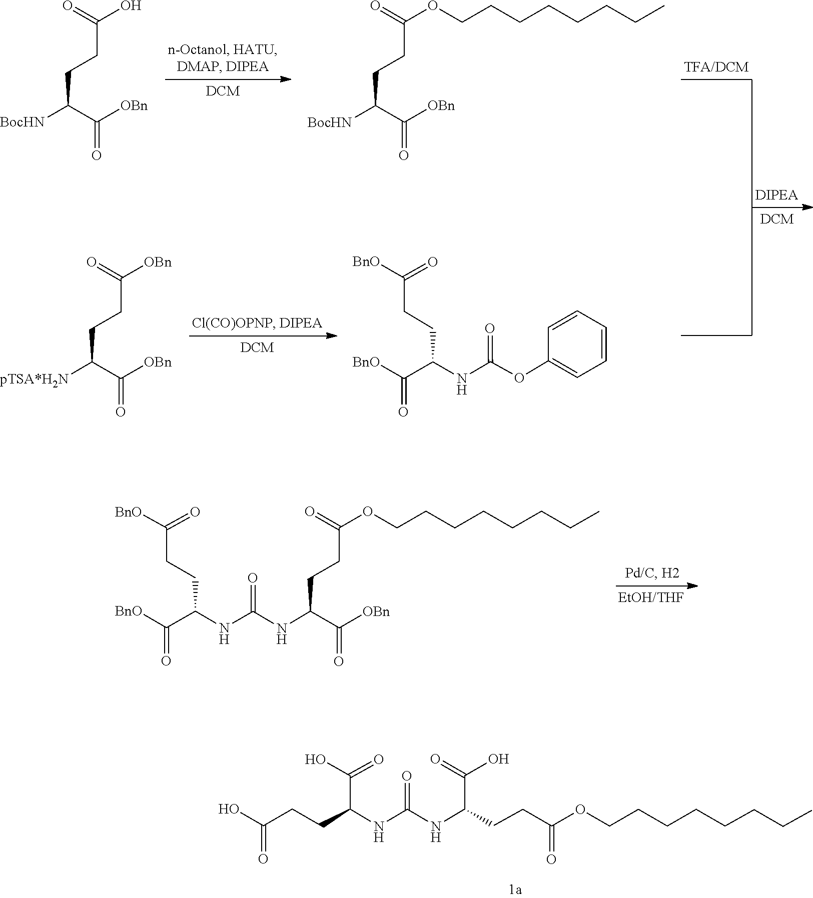

### Step 1: Preparation of (S)-1-benzyl 5-octyl 2-((tert-butoxycarbonyl)amino)pentanedioate

To a stirring solution of Boc-Glu-OBn (1.00 g, 2.96 mmol, 1.00 equiv), n-octanyl alcohol (699 μL, 4.44 mmol, 1.50 equiv), DIPEA (1.54 mL, 8.88 mmol, 3.00 equiv), DMAP (36.2 mg, 0.296 mmol, 10 mol %) in 29.6 mL of DCM was added HATU (1.35 g, 3.55 mmol, 1.20 equiv). The reaction was allowed for 5 h at room temperature before it was diluted with 100 mL of DCM, washed with 30 mL of 2M HCl (aq), 30 mL of water, 30 mL of brine, dried over Na₂SO₄, and filtered. The solution was concentrated under reduced pressure. The desired product was further purified by silica chromatography (5-85% EtOAc in Pet. Ether) to yield a white solid (1.04 g, 81.1%).

Shielding agents 1m and 1n were prepared according to the same method using 1-butanol and 1-dodecanol in place of 1-octanol, respectively.

### Step 2: Preparation of (S)-dibenzyl 2-((phenoxycarbonyl)amino)pentanedioate

In a 100 mL round-bottom flask, Dibenzyl L-glutamate para-toluenesulfonate (5.00 g, 10.0 mmol, 1.00 equiv) and 4-nitrophenyl chloroformate (1.64 g, 10.5 mmol, 1.05 equiv) were dissolved in 30.3 mL of dichloromethane at 0° C. and was stirred under argon for 30 min. Diisopropylethylamine (3.80 mL, 22.0 mmol, 2.20 eq.) was added drop-wise at 0° C. and the reaction mixture was stirred for 5 minutes before it was allowed to warm to room temperature and stirred for an additional 30 minutes. The reaction mixture was then concentrated to a thick light yellow oil. The product was further purified via silica chromatography (0-55% ethyl acetate in petroleum ether) to yield the desired product as a white solid (3.54 g, 78.1%).

### Step 3: Preparation of (S)-dibenzyl 2-(3-((S)-1-(benzyloxy)-5-(octyloxy)-1,5-dioxopentan-2-yl)ureido)pentanedioate

(S)-1-benzyl 5-octyl 2-((tert-butoxycarbonyl)amino)pentanedioate (500mg, 1.11 mmol, 1.00 equiv) was dissolved in dry DCM (5.00 mL). The solution was cooled down to 0° C. and TFA (5.00 mL) was added and the reaction mixture was allowed to slowly heated up to room temperature and stirred for 30 min. DCM and TFA were evaporated in vacuo and the residue was dissolved in toluene (2 mL×3) and coevaporated to remove traces of TFA. The crude product was dissolved in 2.22 mL of DCM and added slowly to a stirring solution of EC3517 (496 mg, 1.11 mmol, 1.00 equiv) dissolved in 2.22 mL of DCM at 0° C. Diisopropylethylamine (424 μL, 2.44 mmol, 2.20 equiv) was added drop-wise at 0° C., and the reaction mixture was stirred for 30 minutes before it was allowed to warm to room temperature. The reaction mixture was stirred for one hour at room temperature before the reaction was concentrated under reduced pressure. The product was extracted from 50 mL of water with DMC (25 mL×3). The combined organic layers were washed with brine (25 mL), dried over Na₂SO₄, filtered, and concentrated. The product was further purified by silica gel chromatography (10-100% EtOAc in Pet. Ether) to yield the desired product at a thick oil (678 mg, 87.0%).

### Step 4: Preparation of (S)-2-(3-((S)-1-carboxy-4-(octyloxy)-4-oxobutyl)ureido)pentanedioic acid (1a)

Preparation of (S)-dibenzyl 2-(3-((S)-1-(benzyloxy)-5-(octyloxy)-1,5-dioxopentan-2-yl)ureido)pentanedioate (441 mg, 0.628 mmol, 1.00 equiv) was dissolved in 6.28 mL of THF/Methanol (3:2). 10% Pd/C (66.6 mg, 0.0628 mmol, 10 mol %) was added under a strong stream of argon. The head space was evacuated and back filled with argon followed by hydrogen (×2). The reaction mixture was then stirred at room temperature for 6 h. The crude product was filtered through a 45 μm Nylon/Fiberglass membrane and concentrated. The product was further purified by reverse phase chromotograph (0-30% ACN in 0.1% TFA aqueous buffer). After two days of lyophilization the desired product was obtained as a white solid (210 mg, 77.3%).

### Example 2

### Preparation of Compound 1c

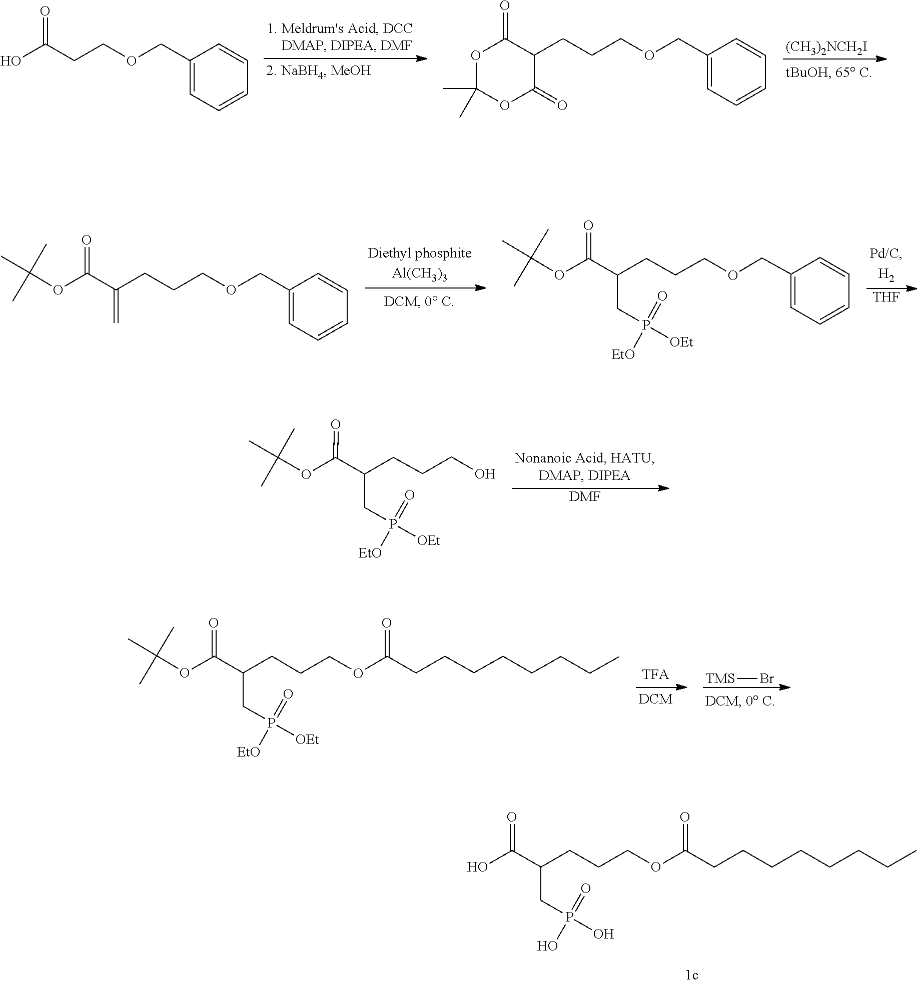

### Step 1: Preparation od 5-(3-(benzyloxy)propyl)-2,2-dimethyl-1,3-dioxane-4,6-dione

To a solution of 3-hydroxybenzyl propionic acid (2.00 g, 10.3 mmol, 1.00 equiv), Meldrum's Acid (2.08 g, 14.4 mmol, 1.40 equiv), diisopropylethylamine (DIPEA) (5.01 mL, 28.8 mmol, 2.8 equiv), 4-(dimethylamino)pyridine (DMAP) (159 mg, 1.30 mmol, 10 mol %) in 103 mL of DCM was added dicyclohexylcarbodiimide (DCC) (2.66 g, 12.9 mmol, 1.25 equiv) portion-wise over 1 h at 0° C. The reaction was allowed to warm to room temperature and stirred overnight (16 h) at room temperature. The white precipitate was filtered off, and the filtrate was washed with 10% KHSO₄ (aq) three times, brine, dried over Na₂SO₄, and filtered. The solution was acidified with acetic acid (7.08 mL, 124 mmol, 12 equiv) at −10° C., and sodium borohydride (NaBH₄) (584 mg, 15.45 mmol, 1.5 equiv) was added portion-wise over the period of 1 h. The reaction mixture was stirred −10° C. overnight (16 h), quenched with water, washed with bring, dried over Na₂SO₄, filtered, and concentrated under reduced pressure. The desired product was further purified by silica chromatography (5-50% EtOAc in Pet. Ether) to yield a white solid (2.47 g, 82%). LC/MS and ¹H NMR spectra analysis agreed with the assigned structure of the desired product.

### Step 2: Preparation of tert-butyl 5-(benzyloxy)-2-methylenepentanoate

5-(3-(benzyloxy)propyl)-2,2-dimethyl-1,3-dioxane-4,6-dione (1.25 g, 4.28 mmol, 1.00 equiv) and Eschenmoser's salt (N,N-dimethylmethyleneiminium iodide) (1.97 g, 10.7 17.2 mmol, 2.50 equiv) were added to a dry round-bottom flask. The head space was evacuated and back-filled with argon. 42.8 mL of absolute tert-butyl alcohol (tBuOH) was added to the solids. The reaction mixture was heated to 65° C. and stirred for 30 h. The reaction was concentrated under high vacuum and the residue was loaded onto silica column and purified by chromatography (0-80% EtOAc in Pet. Ether) to yield the desired product as a clear oil (856 mg, 72.5%). LC/MS and ¹H NMR spectra analysis agreed with the assigned structure of the desired product.

### Step 3: Preparation of tert-butyl 5-(benzyloxy)-2-((diethoxyphosphoryl)methyl)pentanoate

A solution of 2M trimethylaluminum in hexanes (5.80 mL, 2.90 mmol, 1.00 equiv) was added drop-wise to a stirring solution of diethyl phosphite (373 μL, 2.90 mmol, 1.00 equiv) in 41.4 mL of dichloromethane (DCM) at 0° C. The reaction mixture was stirred at 0° C. for 30 min. A solution of tert-butyl 5-(benzyloxy)-2-methylenepentanoate (800 mg, 2.90 mmol, 1.00 equiv) in 7.25 mL of dichloromethane slowly, and the reaction mixture was then allowed to warm to room temperature. The reaction mixture was stirred overnight (17 h) at room temperature. The reaction was quenched with 10 mL of 2M HCl (aq) and extracted with diethyl ether (10 mL×3). The organic layers were combined, washed with water, brine, dried over Na₂SO₄, filtered, and concentrated under reduced pressure. The product was further purified via silica chromatography (10-85% EtOAc in Pet. Ether) to yield the desired product as a clear oil (906 mg, 79.7%). LC/MS and ¹H NMR spectra analysis agreed with the assigned structure of the desired product.

### Step 4: Preparation of tert-butyl 2-((diethoxyphosphoryl)methyl)-5-hydroxypentanoate

tert-butyl 5-(benzyloxy)-2-((diethoxyphosphoryl)methyl)pentanoate (450 mg, 1.09 mmol, 1.00 eq) was dissolved in 10.9 mL of tetrahydrofuran (THF), and argon was bubbled through the solution for 15 min. 10% Pd/C (57.7 mg, 0.055 mmol, 5 mol %) was added under a strong stream of argon. The head space was evacuated and back filled with argon followed by hydrogen (x 2). The reaction mixture was then stirred at room temperature for 4 h. The reaction mixture was then filtered through a pad of celite and wash with 10 mL of dichloromethane. The solution was then concentrated and the residue was held under high vacuum for 1 h to yield the desired product as a colorless oil. The crude product was used without further purification.

### Step 4: Preparation of 5-(tert-butoxy)-4-((diethoxyphosphoryl)methyl)-5-oxopentyl nonanoate

To a stirring solution of nonanoic acid (130 μL, 0.743 mmol, 1.20 equiv), tert-butyl 2-((diethoxyphosphoryl)methyl)-5-hydroxypentanoate (200 mg, 0.619 mmol, 1.00 equiv), DIPEA (301 μL, 1.73 mmol, 2.8 equiv), DMAP (8.0 mg, 0.0619 mmol, 10 mol %) in 7.43 mL of DCM was added HATU (306 mg, 0.805 mmol, 1.30 equiv). The reaction was allowed for 5 h at room temperature before it was diluted with 20 mL of DCM, washed with 10 mL of 2M HCl (aq), 10 mL of water, 10 mL of brine, dried over Na₂SO₄, and filtered. The solution was concentrated under reduced pressure. The desired product was further purified by silica chromatography (5-75% EtOAc in Pet. Ether) to yield a white solid (255 mg, 58.5%). LC/MS and ¹H NMR spectra analysis agreed with the assigned structure of the desired product.

### Step 6: Preparation of 5-(nonanoyloxy)-2-(phosphonomethyl)pentanoic acid (1c)

2.2 mL of trifluoroacetic acid (TFA) was added slowly to the stirring solution of 5-(tert-butoxy)-4-((diethoxyphosphoryl)methyl)-5-oxopentyl nonanoate (200 mg, 0.432 mmol, 1.00 equiv) in 2.2 mL of DCM at 0° C. The reaction was allowed to heat up to room temperature and stirred for 2 h. The solvent was evaporated under reduced, and the residue was brought up in toluene and concentrated under high vacuum (x 3). The crude product was dissolved in 4.32 mL anhydrous DCM, and bromotrimethylsilane (342 μL, 2.59 mmol, 6.00 equiv) was added dropwise at 0° C. The reaction mixture was stirred at 0° C. for 1 h, and then was slowly allowed warmed to room temperature. It was then stirred overnight (12 h) and was concentrated under reduced pressure, and the residue was brought up in toluene and concentrated under high vacuum (×3). The resulting residue was dissolved in ACN/H₂O (4:1, 5 mL) and stirred for 30 min. The reaction was concentrated and loaded onto a C18 column and purified by reverse phase chromotograph (0-35% ACN in 0.1% TFA aqueous buffer). After two days of lyophilization the desired product was obtained as a colorless oil (94 mg, 62.0%). LC/MS and ¹H NMR spectra analysis agreed with the assigned structure of the desired product.

### Example 3

### Preparation of Compound 1d

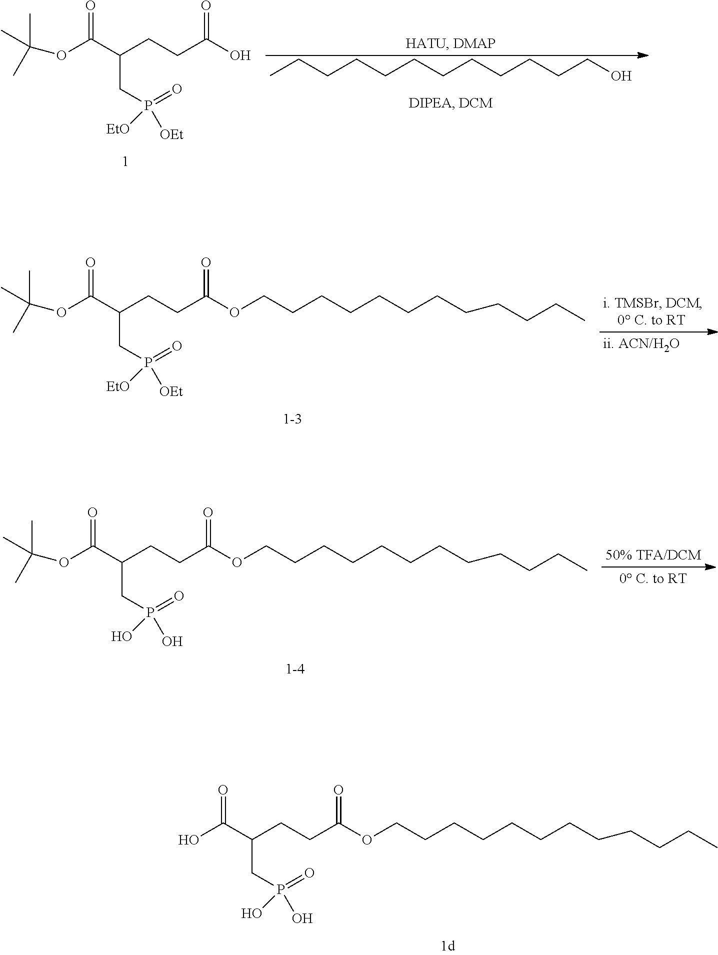

### Step 1: Preparation of 1-(tert-Butyl)-5-dodecyl-2-((diethoxyphosphoryl)methyl)pentanedioate (1-3)

To a solution of compound 1 (0.092 g, 0.27 mM) which was prepared according to the methods described in Nedelcovych 2017, in dry DCM (3 mL) was added 1-dodecanol (0.101 g, 0.54mM) and DIPEA (0.142 mL, 0.82 mM) respectively. HATU (0.124 g, 0.33 mM) and DMAP (3.32 mg, 0.03 mM) were added. The reaction was allowed to stir at RT for 1 h. LCMS analysis (20 mM NH4HCO3, pH 7.4) indicated that the reaction was complete. The reaction mixture was concentrated and dried. Residue was purified using combiflash (SiO₂) column eluting with 0-100% ethyl acetate in petroleum ether to yield pure 1-3 (0.107 g, 78%).

### Step 2: Preparation of 5-Dodecoxy-2-(tert-Butoxycarbonyl)-5-oxopentyl phosphonic acid (1-4)

To a solution of 1-3 (0.096 g, 0.19 mM) in dry DCM (3 mL), under Argon blanket, at 0° C., was added TMSBr (0.116 g, 0.76 mM) very slowly over 5 min. The reaction was warmed to RT over 2 h and stirred for 18 h. LCMS analysis (20 mM NH₄HCO₃, pH 7.4) indicated that the reaction was complete. DCM was removed, TMSBr was co-evaporated with toluene (3×3 mL) and dried. Residue was dissolved in acetonitrile/water (5:1; 6 mL) and stirred at RT for 30 min. Concentrated under reduced pressure, co-evaporated with toluene (3×3 mL) and dried. The crude product des-ethyl 1-4 (0.086 g, quantitative) was directly used for next reaction.

### Step 3: Synthesis of 5-Dodecoxy-5-oxo-2-(phosphonomethyl)pentanoic acid (1d)

To a solution of des-ethyl 1-4 (0.086 g, 0.19 mM) in dry DCM (2 mL), at 0° C., was added trifluroacetic acid (2 mL) very slowly over 5 min. The reaction was warmed to RT and stirred for 2 h. LCMS analysis (20 mM NH₄HCO₃, pH 7.4) indicated that the reaction was complete. TFA/DCM was removed and dried. Residue was dissolved in DMSO and purified on Biotage column (C₁₈; 0-50% acetonitrile and 0.1% TFA in water). Pure fractions were combined, acetonitrile was removed and freeze dried to yield 1d (0.062 g, 83%).

Compound 1b was prepared according to the same method as compound 1d, except that 1-octcanol was used in place of 1-dodecanol.

### Example 4

### Preparation of Compound 1e

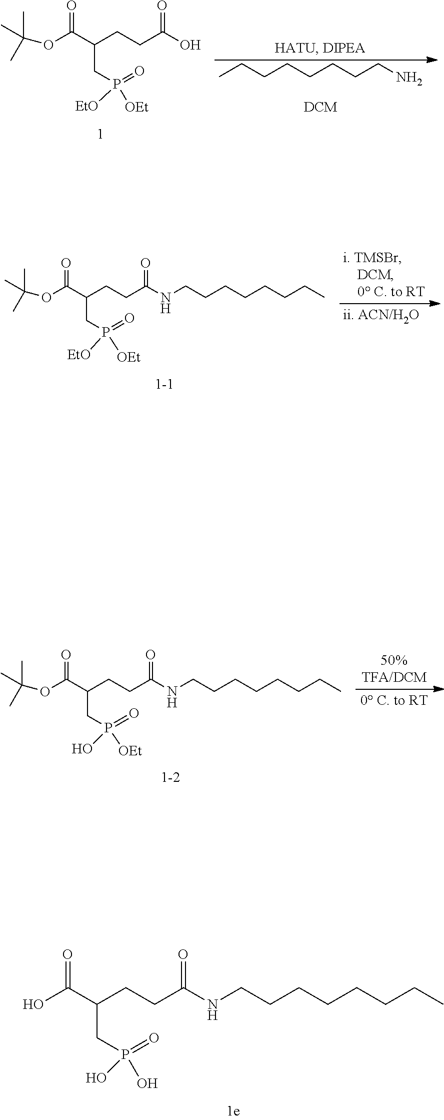

### Step 1: Preparation of 1-(tert-Butyl)-2-((diethoxyphosphoryl)methyl)-5-(octylamino)-5-oxopentanoate (1-1)

To a solution of Compound 1 (0.100 g, 0.30 mM) which was prepared according to the methods described in Nedelcovych 2017, cited above, in dry DCM (3 mL) was added 1-octyl amine (0.077 g, 0.59mM) and DIPEA (0.155 mL, 0.892 mM) respectively. HATU (0.135 g, 0.36 mM) was added. The reaction was allowed to stir at RT for 1 h. LCMS analysis (20 mM NH₄HCO₃, pH 7.4) indicated that the reaction was complete. The reaction mixture was concentrated and dried. Residue was purified using combiflash (SiO₂) column eluting with 0-100% ethyl acetate in petroleum ether to yield pure 1-1 (0.088 g, 66%).

### Step 2: Preparation of 5-(Octylamino)-2-(tert-Butoxycarbonyl)-5-oxopentyl phosphonic acid (1-2)

To a solution of compound 1-1 (0.088 g, 0.20 mM) in dry DCM (3 mL), under Argon blanket, at 0° C., was added TMSBr (0.119 g, 0.78 mM) very slowly over 5 min. The reaction was warmed to RT over 2 h and stirred for 24 h. LCMS analysis (20 mM NH₄HCO₃, pH 7.4) indicated that the reaction was complete. DCM was removed, TMSBr was co-evaporated with toluene (3×5 mL) and dried. Residue was dissolved in acetonitrile/water (5:1; 6 mL) and stirred at RT for 30 min. Concentrated under reduced pressure, co-evaporated with toluene (3×5 mL) and dried. The crude product compound 1-2 (0.077 g, quantitative) was directly used for next reaction.

### Step 3: Preparation of 5-(Octylamino)-5-oxo-2-(phosphonomethyl)pentanoic acid (1e)

To a solution of des-ethyl 1-2 (0.077 g, 0.20 mM) in dry DCM (2.5 mL), at 0° C., was added trifluroacetic acid (2.5 mL) very slowly over 5 min. The reaction was warmed to RT and stirred for 20 h. LCMS analysis (20 mM NH₄HCO₃, pH 7.4) indicated that the reaction was complete. TFA/DCM was removed and dried. Residue was dissolved in DMSO and purified on Biotage column (C₁₈; 0-50% acetonitrile and 0.1% TFA in water). Pure fractions were combined, acetonitrile was removed and freeze dried to yield compound 1e (0.040 g, 60%).

### Biology Examples:

### Example 5

### Biodistribution of Shielding Agents of the Present Disclosure

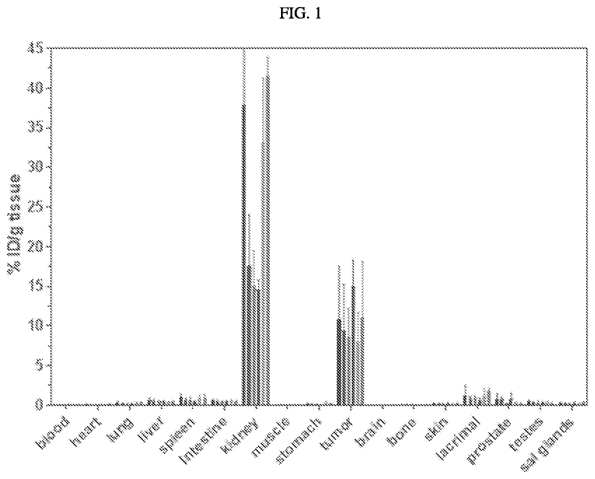

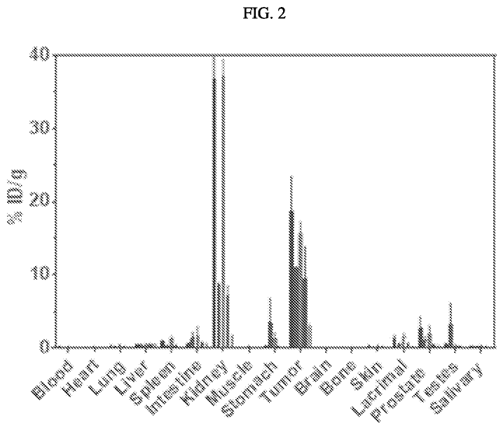

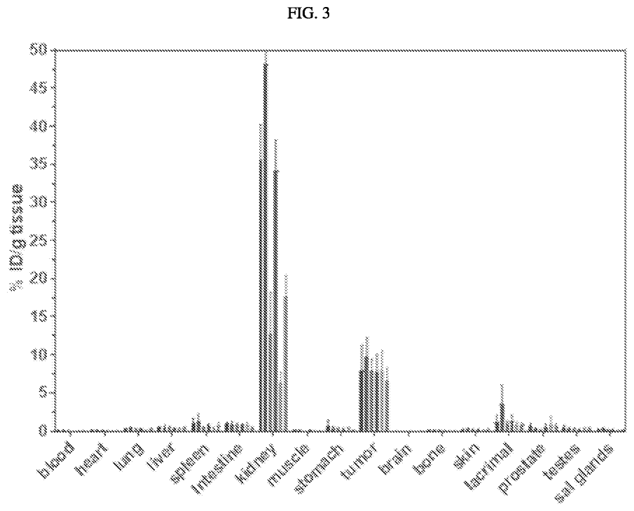

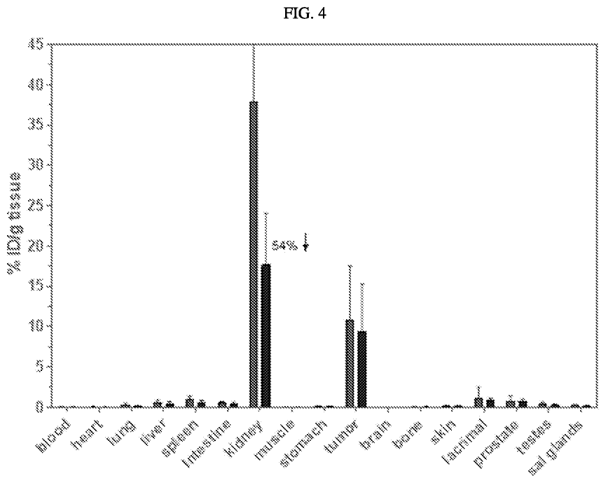

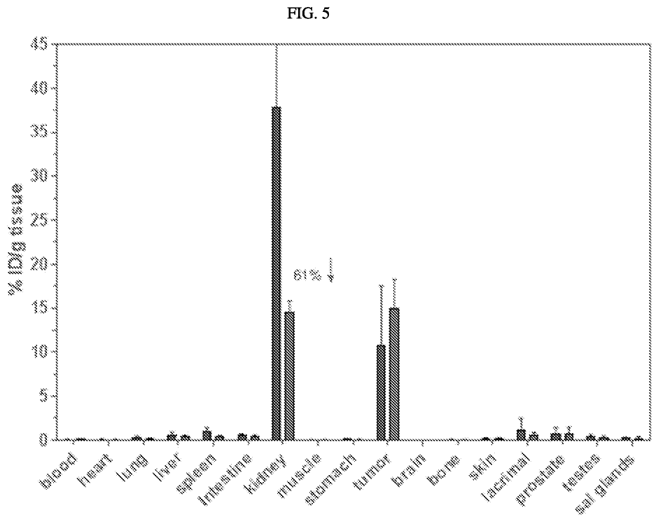

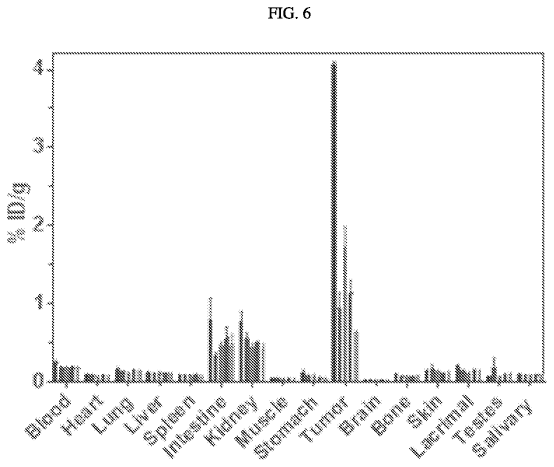

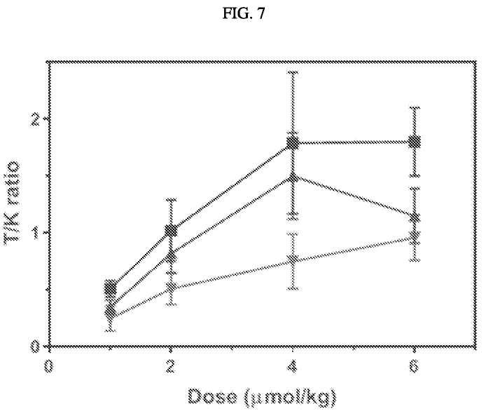

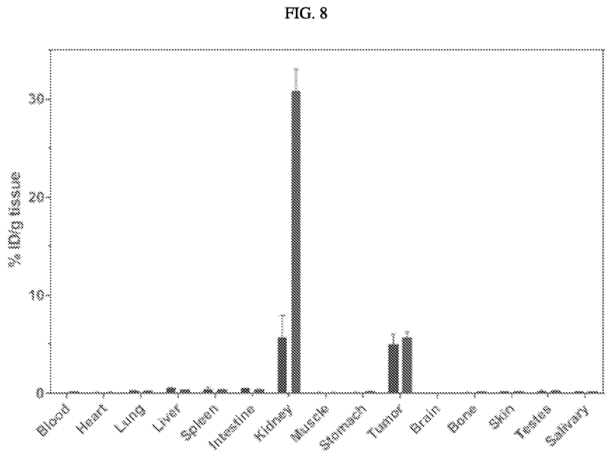

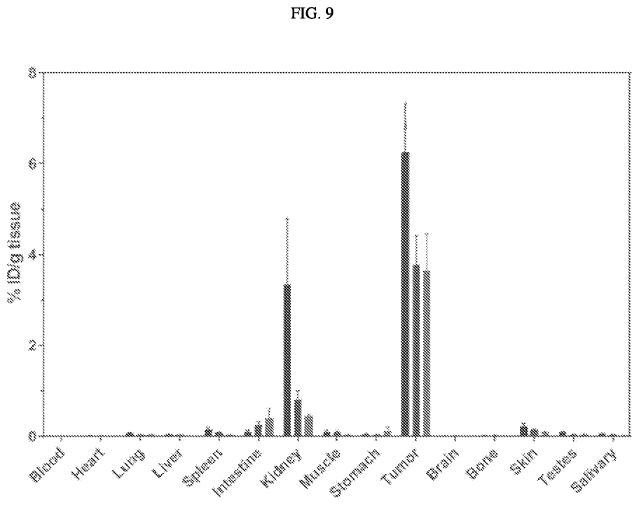

The shielding agents of the present disclosure were administered in combination with an imaging agent of the disclosure, and the biodistribution was analyzed. Results are shown in FIG. 1-FIG. 9.

1. A compound selected from the group consisting of

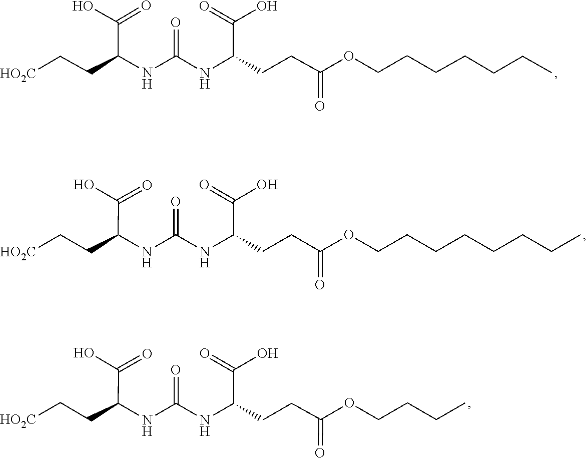 [SMILES: CCCCCCCOC(=O)CC[C@H](NC(=O)N[C@@H](CCC(=O)O)C(=O)O)C(=O)O ; CCCCCCCCOC(=O)CC[C@H](NC(=O)N[C@@H](CCC(=O)O)C(=O)O)C(=O)O ; CCCCOC(=O)CC[C@H](NC(=O)N[C@@H](CCC(=O)O)C(=O)O)C(=O)O]

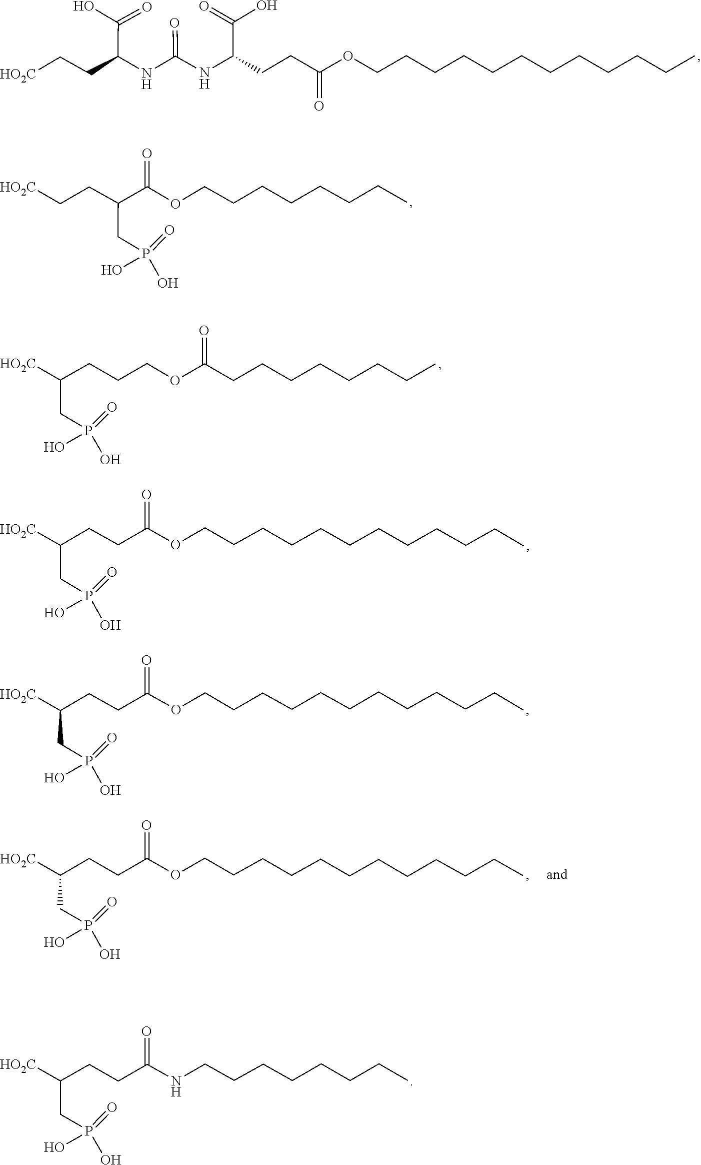

2. A method for treating a cancer in a patient comprising administering a therapeutically effective amount of a radiolabeled therapeutic in combination with a an effective amount of a shielding agent.

3. The method of claim 2, wherein the radiolabeled therapeutic is Compound Ia-Lu of the formula 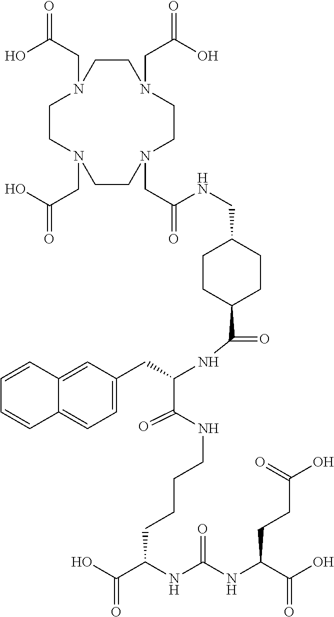 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O]
wherein ¹⁷⁷Lu is coordinated thereto or Compound Ia-Ac of the formula 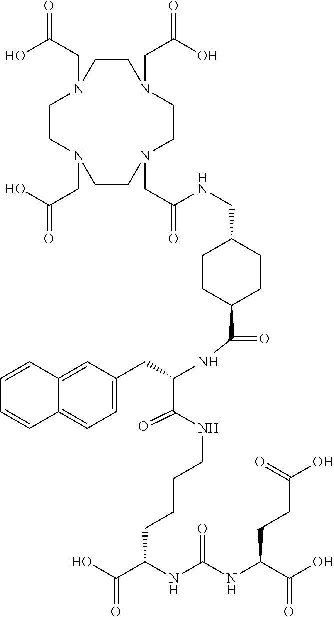 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O]
wherein ¹⁷⁷Lu is coordinated thereto.

4. The method of claim 3, wherein the cancer is a prostate cancer.

5. The method of claim 4, wherein the cancer is a metastatic prostate cancer.

6. The method of claim 4, wherein the cancer is a metastatic castration-resistant prostate cancer.

7. The method of claim 4, wherein the shielding agent is selected from the group consisting of

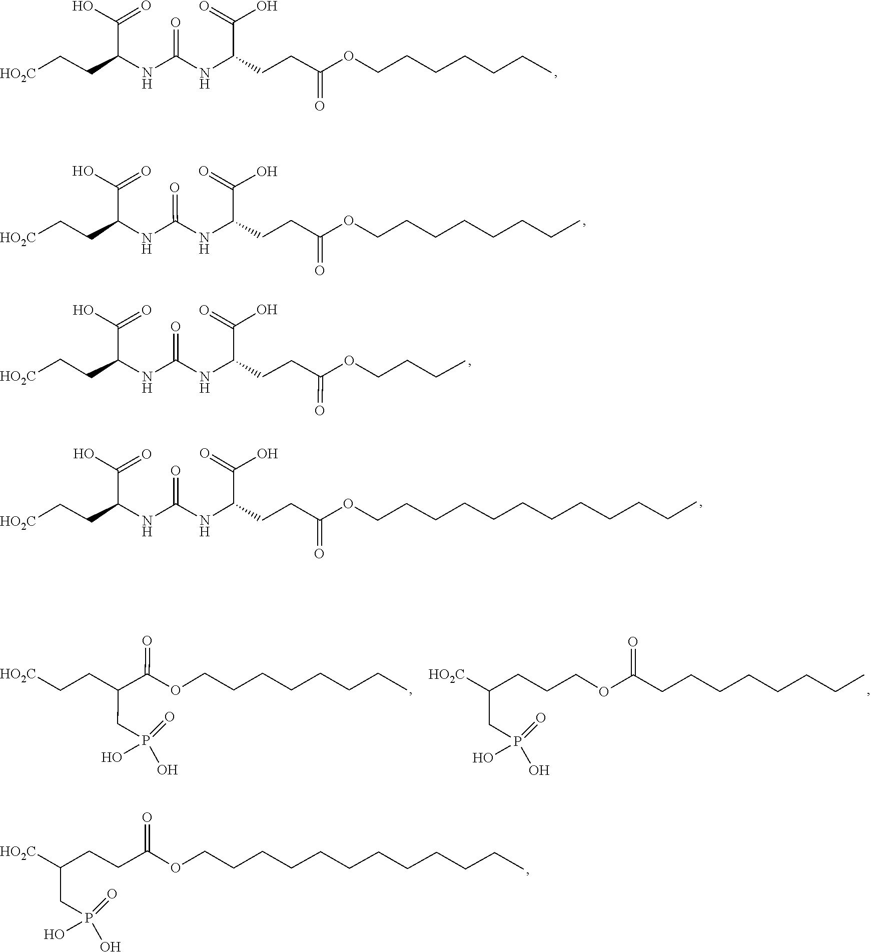

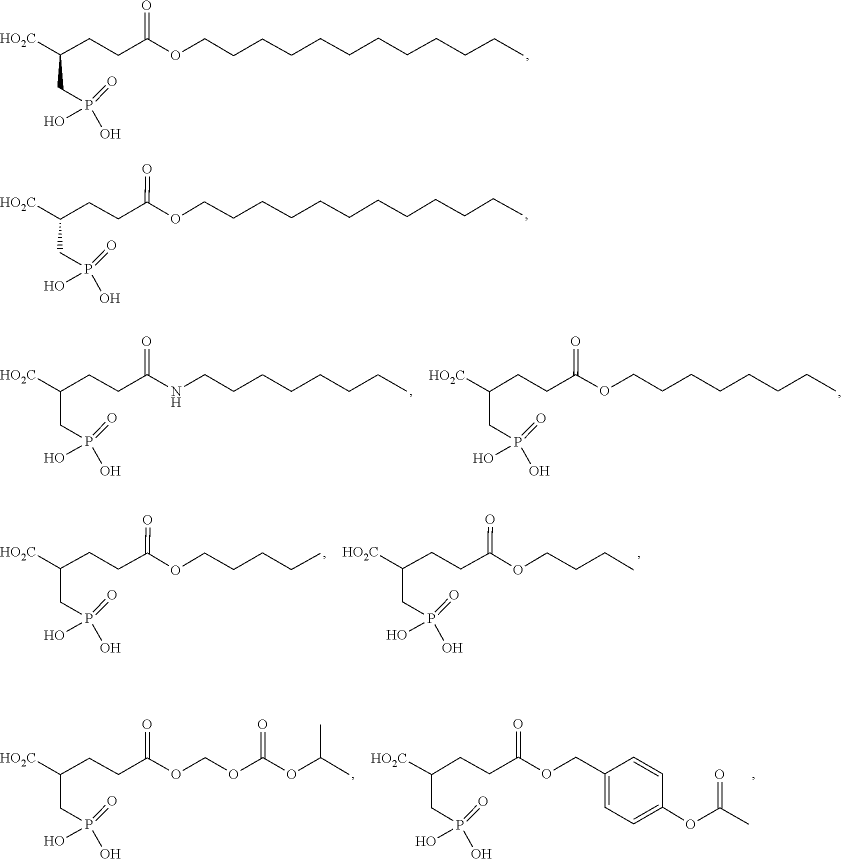

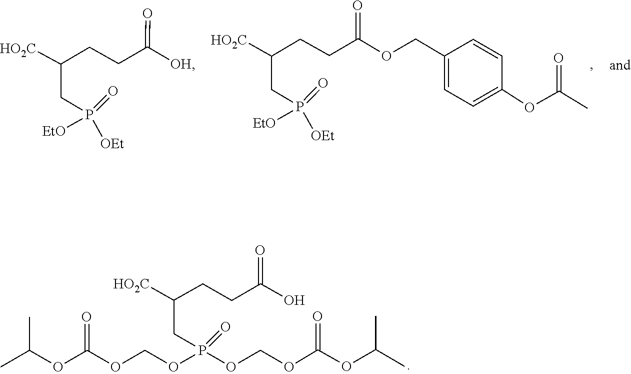 [SMILES: CCOP(=O)(CC(CCC(=O)O)C(=O)O)OCC ; CCOP(=O)(CC(CCC(=O)OCc1ccc(OC(C)=O)cc1)C(=O)O)OCC ; CC(C)OC(=O)OCOP(=O)(CC(CCC(=O)O)C(=O)O)OCOC(=O)OC(C)C]

8. The method of claim 4, wherein the shielding agent is selected from the group consisting of

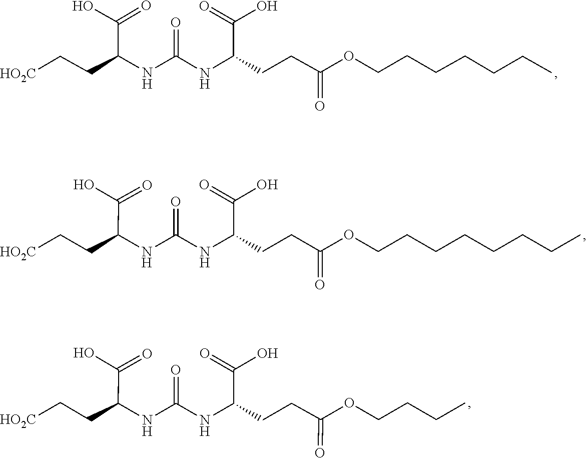 [SMILES: CCCCCCCOC(=O)CC[C@H](NC(=O)N[C@@H](CCC(=O)O)C(=O)O)C(=O)O ; CCCCCCCCOC(=O)CC[C@H](NC(=O)N[C@@H](CCC(=O)O)C(=O)O)C(=O)O ; CCCCOC(=O)CC[C@H](NC(=O)N[C@@H](CCC(=O)O)C(=O)O)C(=O)O]

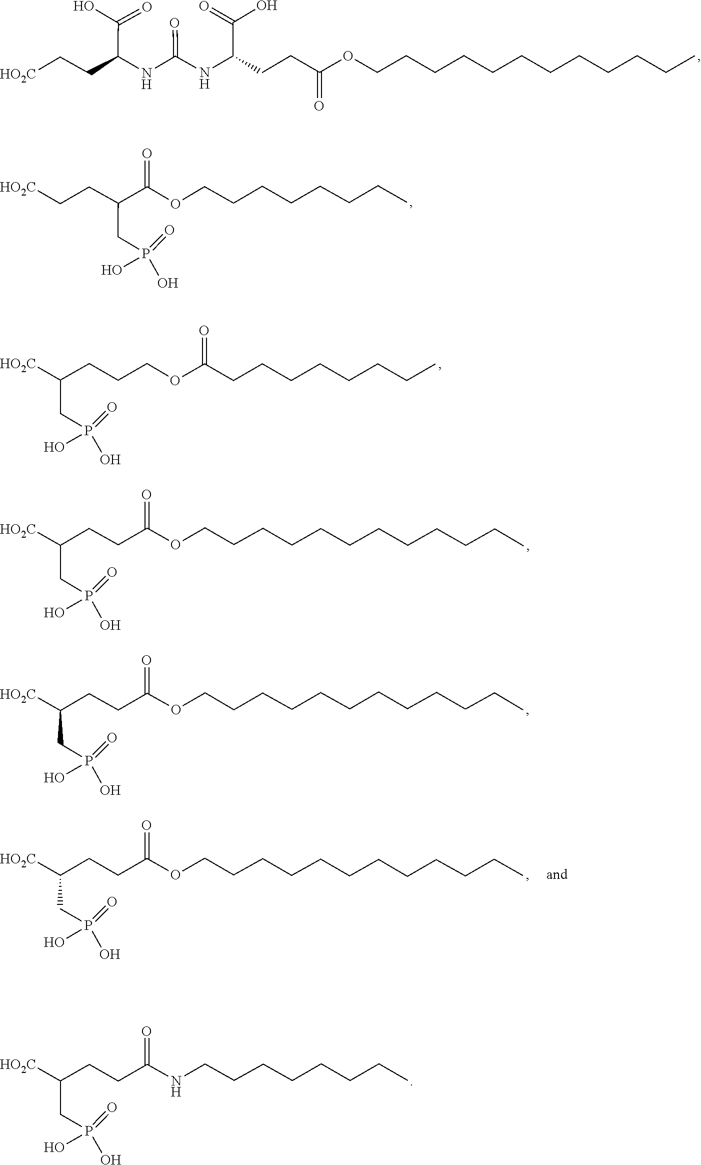

9.-18. (canceled)

19. A method for imaging a cancer in a patient comprising administering an effective amount of an imaging conjugate in combination with an effective amount of a shielding agent.

20. The method of claim 19, wherein the imaging is a ^{99m}Tc labelled imaging conjugate of the formula 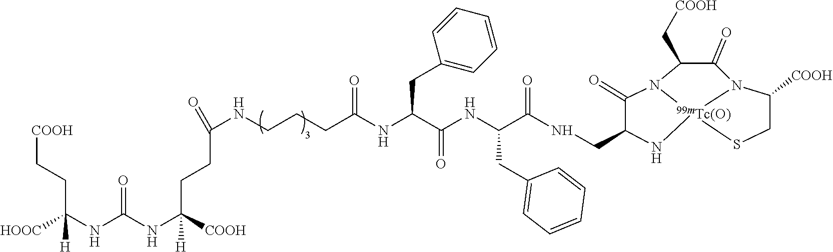
or ⁶⁷Ga or⁶⁸Ga labelled an imaging conjugate of the formula 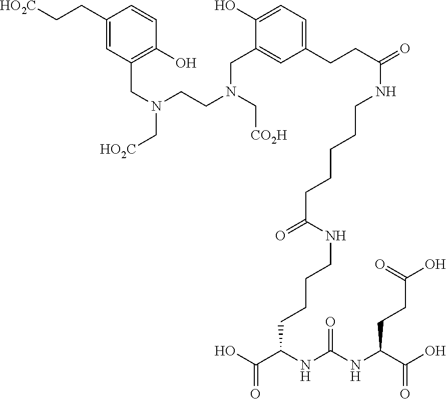 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]
wherein ⁶⁷Ga or ⁶⁸Ga is coordinated thereto.

21. The method of claim 19, wherein the cancer is a prostate cancer.

22. The method of claim 21, wherein the cancer is a metastatic prostate cancer.

23. The method of claim 21, wherein the cancer is a metastatic castration-resistant prostate cancer.

24. The method of claim 21, wherein the shielding agent is selected from the group consisting of

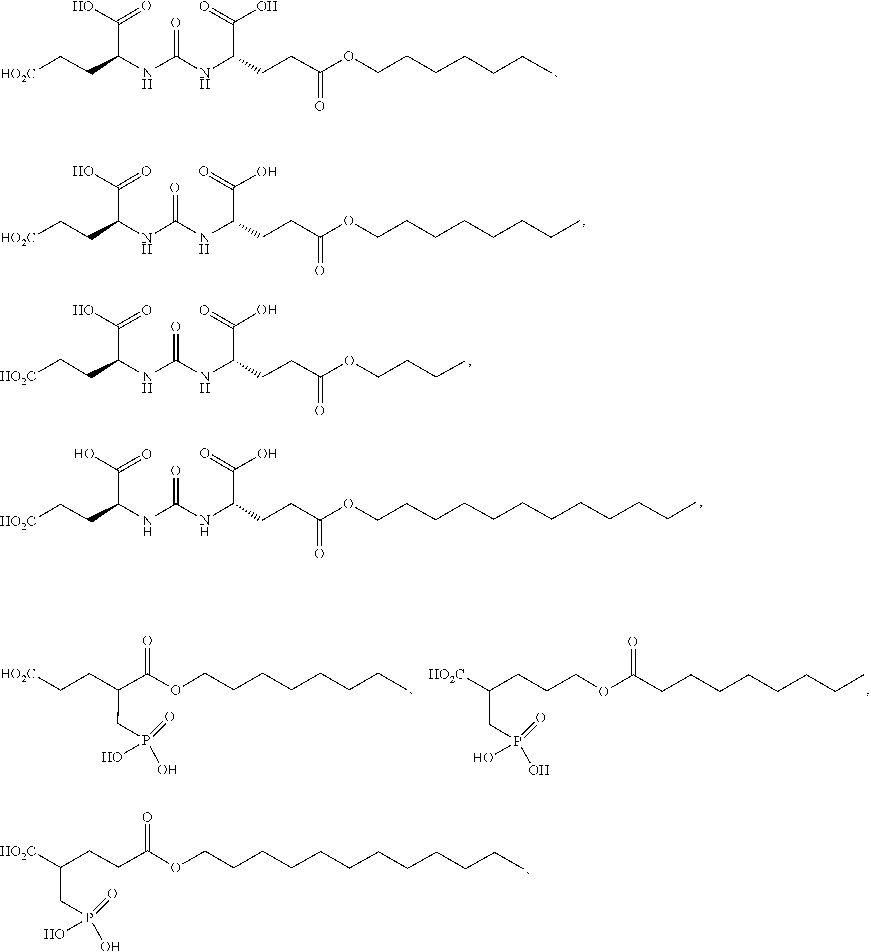

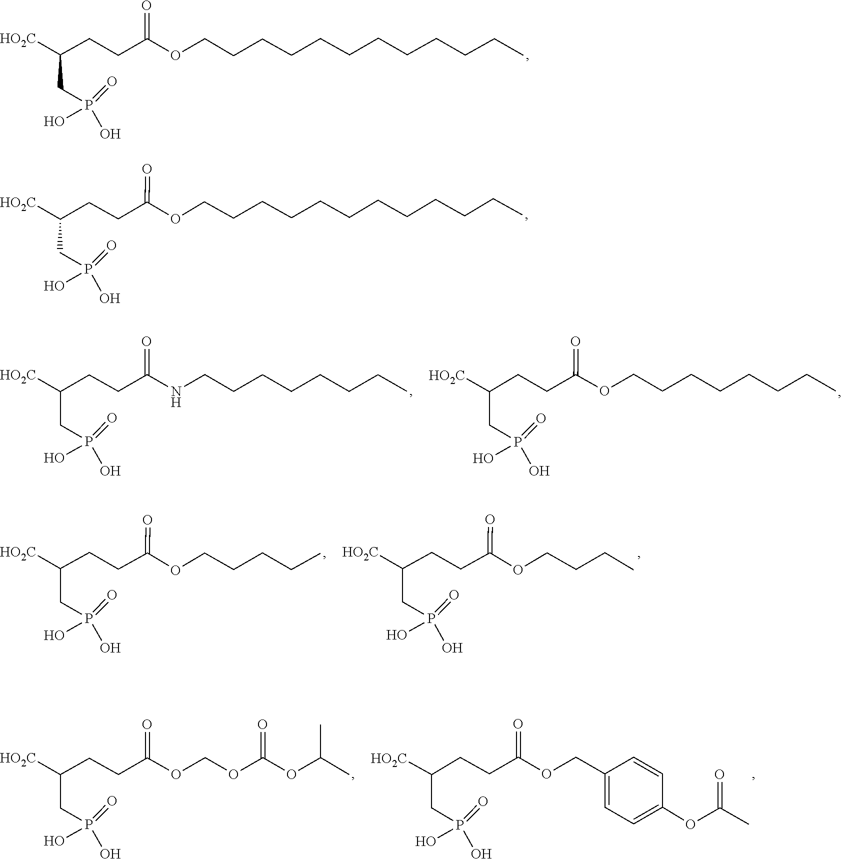

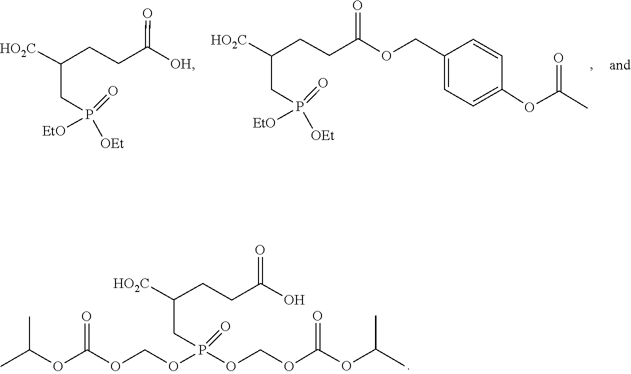 [SMILES: CCOP(=O)(CC(CCC(=O)O)C(=O)O)OCC ; CCOP(=O)(CC(CCC(=O)OCc1ccc(OC(C)=O)cc1)C(=O)O)OCC ; CC(C)OC(=O)OCOP(=O)(CC(CCC(=O)O)C(=O)O)OCOC(=O)OC(C)C]

25. The method of claim 21, wherein the shielding agent is selected from the group consisting of

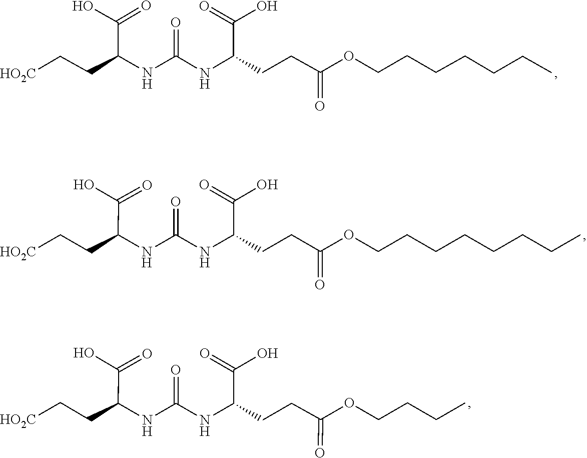 [SMILES: CCCCCCCOC(=O)CC[C@H](NC(=O)N[C@@H](CCC(=O)O)C(=O)O)C(=O)O ; CCCCCCCCOC(=O)CC[C@H](NC(=O)N[C@@H](CCC(=O)O)C(=O)O)C(=O)O ; CCCCOC(=O)CC[C@H](NC(=O)N[C@@H](CCC(=O)O)C(=O)O)C(=O)O]

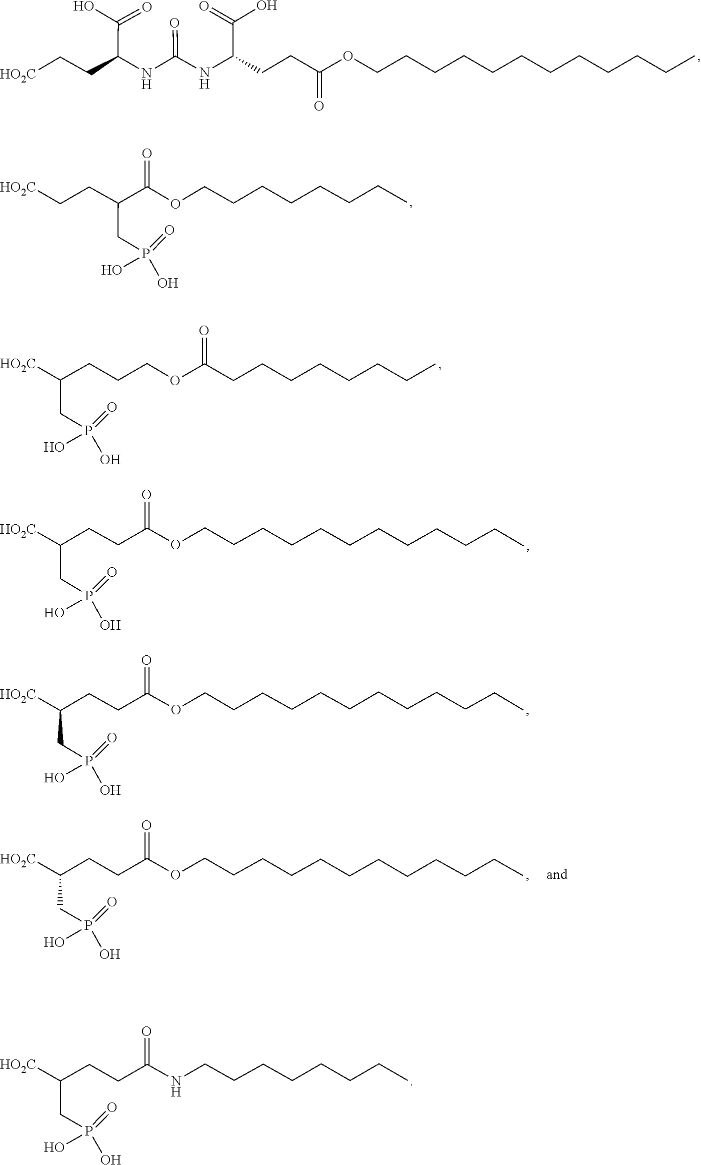

26.-35. (canceled)
